(19) United States
(12) Patent Application Publication
(10) Pub. No.: US 2023/0243818 A1
(43) Pub. Date: Aug. 3, 2023

(54) Low-Cost Rapid Diagnostic Biosensors

(71) Applicants: THE TRUSTEES OF THE UNIVERSITY OF PENNSYLVANIA, Philadelphia, PA (US); STATE UNIVERSITY OF CAMPINAS (UNICAMP), Campinas/SP (BR)

(72) Inventors: Cesar De La Fuente-Nunez, Philadelphia, PA (US); Marcelo Der Torossian Torres, Philadelphia, PA (US); William Reis De Araujo, Campinas/SP (BR)

(21) Appl. No.: 18/296,613

(22) Filed: Apr. 6, 2023

Related U.S. Application Data
(63) Continuation-in-part of application No. PCT/US2021/071789, filed on Oct. 8, 2021.
(60) Provisional application No. 63/089,905, filed on Oct. 9, 2020.
(60) Provisional application No. 63/134,690, filed on Jan. 7, 2021.
(60) Provisional application No. 63/155,963, filed on Mar. 3, 2021.
(60) Provisional application No. 63/489,494, filed on Mar. 10, 2023.

(51) Int. Cl. G01N 33/543; G01N 33/569

(52) U.S. Cl. CPC G01N 33/54373; G01N 33/56983; G01N 33/56994; G01N 2333/165; G01N 2333/035

(57) ABSTRACT

Provided are devices for assessing the presence of SARS-CoV-2 in a biological sample, the devices comprising a substrate comprising a top surface and a back surface; and, an electrode on the top surface of the substrate, wherein the electrode is functionalized with a detection moiety that binds SARS-CoV-2 spike protein. Also disclosed are articles that include such devices, and methods for assessing the presence of SARS-CoV-2 in a biological sample using the disclosed devices. The present disclosure also provides devices for assessing the presence of herpes simplex virus (HSV) in a biological sample comprising a substrate that includes a top surface and a back surface; and, an electrode on the top surface of the substrate, wherein the electrode is functionalized with a detection moiety that binds HSV glycoprotein gD2, such as nectin-1. Also disclosed are articles that include such devices, and methods for assessing the presence of HSV in a biological sample using the disclosed devices.

### CROSS-REFERENCE TO RELATED APPLICATIONS

The present application is the U.S. national stage and is also a continuation-in-part of PCT/US2021/071789, filed Oct. 8, 2021, which claims priority to U.S. Provisional Application No. 63/089,905, filed Oct. 9, 2020, U.S. Provisional Application No. 63/134,690, filed Jan. 7, 2021, and U.S. Provisional Application No. 63/155,963, filed Mar. 3, 2021. The present application also claims priority to U.S. Provisional Application No. 63/489,494, filed Mar. 10, 2023. The entire contents of each of the above-cited patent applications are incorporated herein by reference.

### TECHNICAL FIELD

The present disclosure pertains to devices and methods for detecting infection by a pathogen in a mammalian subject

### BACKGROUND

COVID-19, the severe acute respiratory illness caused by SARS-CoV-2, has led to over 6.57 million deaths worldwide and continues affecting millions of people, primarily in low-income countries, low-resource settings, and communities with low vaccination coverage. Emerging SARS-CoV-2 variants may have harmful interactions with host immunity, as well as increased infectivity, disease severity, and mortality. Low-cost and rapid response technologies that enable accurate, frequent testing of SARS-CoV-2 variants are crucial for outbreak prevention and infectious disease control. Biosensor technologies represent an alternative low-cost approach for detecting infectious diseases including COVID-19. The most widely used substrate for manufacturing electrical circuits and consequently electrodes is the printed circuit board (PCB). PCBs contain Cu, Al, and Sn, consisting of nearly 28% metal. These metals in PCBs are more than 10 times purer than the metals in rich-content minerals. Because PCBs are used extensively and discarded afterward, the recycling of PCBs is not trivial. Moreover, the high percentage of nonmetals in PCBs is around 70%, consisting mostly of thermoset resins and reinforcing materials; these materials pose a particularly challenging recycling problem. The network structure of thermoset resins hinders them from being remelted or reformed. Due to inorganic fillers like glass fiber, with considerably lower fuel efficiency, incineration is not appropriate for treating nonmetals. Nonmetal components of PCBs are mostly disposed of in landfills, which can waste resources and produce significant secondary contamination.

Therefore, there is an urgent need to develop approaches to detect and diagnose both viral and bacterial infections that are also compatible with environmental considerations.

Both types of herpes simplex virus (HSV), HSV-1 and HSV-2, are prevalent in humans, and both cause neonatal infections. Furthermore, these viruses can establish lifelong latency in the sensory neuronal ganglia. Subsequent reactivation of latent virus may cause significant health problems and result in viral transmission to healthy individuals. HSV-1, also known as oral herpes, infects the lips, mouth, eyes, and brain, while HSV-2, also known as genital herpes, is associated mainly with genital infections. Herpes simplex virus type 2 (HSV-2) infection is almost exclusively sexually transmitted.

The World Health Organization (WHO) recently estimated the global prevalence of HSV-1 in individuals aged 0-49 years to be 66.6%, or more than 3.7 billion people who have been infected by HSV-1. Additionally, the WHO estimates the global prevalence of HSV-2, which is transmitted almost exclusively through sexual contact, to include 13.2% of the world's population, or 491.6 million people aged 15-49 years. The attachment of the virus to the cell surface initially involves two glycoproteins on the HSV envelope, glycoprotein C (gC), and to a lesser extent, glycoprotein B (gB). Glycoprotein D (gD), found within the viral envelope, then binds to host cell receptors, initiating a sequence of events that allow HSV to fuse with the host's cell plasma membrane. Studies of the binding of gD to cell surface receptors have led to an understanding of the interaction between human cell receptors and HSV.

Despite the prevalence of HSV-2 infections, there are currently no rapid tests available to detect this infectious agent. Historically, viral culturing has been the main test used for HSV detection in the clinic. However, recently, molecular methods such as polymerase chain reaction (PCR) have been widely used in clinical practice due to their increased sensitivity and selectivity compared to viral culture. Currently, there are very few FDA-cleared molecular tests available for HSV detection. Examples include PCR-based MultiCode-RTx kit, ProbeTec HSV Qx test, and IsoAmp HSV assay with sensitivity and selectivity ranging from 92.4% to 98.4% and 83.7% to 97.0%, respectively. Other commercial serological methods such as immunoblot (IB), ELISA, Western blotting, and chemiluminescence immunoassay (CLIA) have also been used to detect HSV. However, immunoassays rely on the availability of HSV antibodies, and thus, the sensitivity of these tests is influenced by the amount of time since the infection. Indeed, immunoassays display the highest sensitivity when performed at least 21 days after the initial infection and may improve if performed more than 40 days after the primary infection, thus clearly hindering early HSV diagnosis. In addition, these diagnostic methods are time-consuming, costly, and laborious, requiring highly trained staff and sophisticated laboratory infrastructure.

Rapid and accessible diagnostic technologies could improve the management of HSV infections, particularly in low-resource settings and in labor and delivery wards. In fact, several portable devices have been reported as alternative methods for the diagnosis of HSV, and electrochemical detection methods are attractive for developing such devices. Electrochemical detection has adequate sensitivity and selectivity and can be associated with accessible and portable instrumentation. Generally, these portable diagnostic devices are DNA-based biosensors aiming to detect the viral genetic material. Detecting viral DNA or RNA present in biofluids can lead to base-pairing mismatches and hybridization problems that compromise the selectivity of the tests. Moreover, these methods commonly require preconcentration or amplification protocols to achieve the desired sensitivity, decreasing the ability to conduct rapid, frequent, and inexpensive tests.

Rapid and accessible diagnostic technologies constitute promising approaches to help manage HSV-2 infections.

### SUMMARY

Provided herein are devices for assessing the presence of a pathogen, such as, SARS-CoV-2, in a biological sample. The devices can comprise a substrate comprising bacterial cellulose and the substrate can include a top surface and a back surface; and, an electrode on the top surface of the substrate, wherein the electrode is functionalized with a detection moiety, such as one that binds SARS-CoV-2 spike protein, and a chemical cross linker comprising polyethylene glycol (PEG) that enables immobilization of the detection moiety that binds SARS-CoV-2 spike protein on the electrode.

Also provided are wearable articles comprising a device as described herein for assessing the presence of a pathogen, such as, SARS-CoV-2.

The present disclosure also pertains to methods for assessing the presence of SARS-CoV-2 in a biological sample comprising contacting a device according to the present disclosure with the biological sample; exposing the device to an electrical current in order to generate a signal from the device; and, assessing the signal that is generated by the device electrochemical impedance spectroscopy (EIS) in order to determine the absence or presence of SARS-CoV-2 in the biological sample.

Also disclosed herein are devices for assessing the presence of herpes simplex virus (HSV) in a biological sample comprising a substrate that includes a top surface and a back surface; and, an electrode on the top surface of the substrate, wherein the electrode is functionalized with a detection moiety that binds HSV glycoprotein gD2, such as nectin-1. A chemical cross-linker may be present in order to enable immobilization of the detection moiety on the electrode.

Also provided are wearable articles comprising a device as described herein for assessing the presence of HSV.

The present disclosure also pertains to methods for assessing the presence of HSV in a biological sample comprising contacting a device according to the present disclosure with the biological sample; exposing the device to an electrical current in order to generate a signal from the device; and, assessing the signal that is generated by the device electrochemical impedance spectroscopy (EIS) in order to determine the absence or presence of HSV in the biological sample.

Also provided herein are devices for assessing the presence of a pathogen, such as, SARS-CoV-2, in a biological sample. The devices can comprise a substrate comprising a top surface and a back surface; and, an electrode on the top surface of the substrate, wherein the electrode is functionalized with a detection moiety, such as one that binds SARS-CoV-2 spike protein.

Also disclosed are wearable articles comprising a device as described herein.

The present disclosure also pertains to methods for assessing the presence of SARS-CoV-2 in a biological sample comprising contacting a device according to the present disclosure with the biological sample; exposing the device to an electrical current in order to generate a signal from the device; and, assessing the signal that is generated by the device electrochemical impedance spectroscopy (EIS) in order to determine the absence or presence of SARS-CoV-2 in the biological sample.

### BRIEF DESCRIPTION OF THE DRAWINGS

The file of this patent or application contains at least one drawing/photograph executed in color. Copies of this patent or patent application publication with color drawings/photographs will be provided by the Office upon request and payment of the necessary fee.

FIGS. 1A-1E illustrate the fabrication, optimization, and characterization of inventive SARS-CoV-2 electrochemical biosensors using bacterially produced cellulose.

FIG. 2A provides micrographs of a bacterial cellulose substrate at magnifications of 13,000 and 25,000×, respectively, and FIG. 2B provides Raman spectra of the BC substrate (black), BC/carbon ink electrode (red), and BC/carbon ink/G-PEG electrode (green).

FIGS. 3A-3C show the electrochemical behaviour of inventive biodegradable bacterial cellulose-based biosensors.

FIGS. 4A-4C provide potentiometric measurements and dose-response curves for SARS-CoV-2 detection using the present biosensors.

FIGS. 5A-5C illustrate the use of the present biosensors for electrochemical detection of SARS-CoV-2 variants in human NP/OP biofluid samples.

FIG. 6 depicts the results of a selectivity evaluation for inventive biosensors.

FIG. 7 provides a plot illustrating the results of a reproducibility study, showing potential difference (ΔE) obtained for 10 biosensors when incubated with 1×10¹ copies μL⁻¹ of SARS-CoV-2 prepared in VTM medium. A volume of 10 μL of each virus was incubated on the biosensor surface for 7 minutes before the potentiometric measurements were made. The relative standard deviation (RSD) was 3.78% in these assays.

FIG. 8 provides a plot illustrating the results of an investigation of the potential stability of the inventive HSV biosensors. Biosensors were tested for stability for 1 hour using 0.1 mol L−1 PBS as a blank sample (black line) and with VTM as a blank sample (red line) to evaluate the best medium for sample analysis. PBS presented a stable response after the first 60 s, whereas VTM presented a drift potential response over a long period of use (>500s).

FIG. 9A provides a schematic representation of the HSV sensing using a electrochemical biosensor according to the present disclosure, and FIG. 9B depicts the functionalization and optimization steps of the electrochemical biosensor.

FIGS. 10A-10D show the results of a characterization of an inventive HSV electrochemical biosensor.

FIG. 11A illustrates the functionalization steps for the preparation of an inventive HSV biosensor, and FIGS. 11B and 11C depict the result of an electrochemical characterization thereof.

FIG. 12A provides Nyquist plots for increased concentration of gD2, FIG. 12B provides a dose-response curve extracted from Nyquist plots as a function of the logarithm of the gD2 concentration, FIG. 12C shows Nyquist plots for titered HSV-2 viral solution, and FIG. 12D provides a dose-response curve extracted from Nyquist plots as a function of the logarithm of the HSV-2 viral loads.

FIG. 13 provides the results of a study of the detection of HSV-2 in biofluid samples from guinea pigs.

FIGS. 14A and 14B provide normalized analytical curves plotted to calculate the limit of detection using the four-parameter logistic 4PL method. FIG. 14A provides a dose-response curve obtained by normalizing RCT values extracted from Nyquist plots as a function of the logarithm of the gD2 concentration, and FIG. 14B provides a dose-response curve obtained from normalized RCT values extracted from Nyquist plots as a function of the logarithm of the HSV-2 viral loads.

FIG. 15 depicts the results of an experiment to evaluate the effect of pH on the analytical response of an inventive HSV biosensor.

FIG. 16 illustrates the results of a reproducibility study of the inventive HSV biosensors.

FIG. 17 depicts the results of an investigation concerning the stability of inventive HSV biosensors under various temperature conditions.

FIG. 18 illustrates the results of a selectivity study of inventive HSV biosensors.

FIG. 19 illustrates the detection capabilities of a device according to the present disclosure that is configured as a bandage.

FIG. 20 depicts the in vitro detection of infectious agents by a device according to the present disclosure.

FIG. 21 illustrates molecular dynamic simulations of the region of the SARS CoV-2 viral spike protein that binds to the human ACE2 protein.

FIG. 22 illustrates a process by which the inventive devices may be used.

FIG. 23 depicts the concept under which the inventive devices are used for rapid SARS-CoV-2 detection.

FIG. 24 provides the results of a real time assessment for diagnosing COVID-19, in which detection=pg-ng of virus.

FIG. 25 depicts elements of point-of-care detection of SARS-CoV-2 using the DETECT 1.0 system.

FIG. 26 illustrates the characterization and calibration of an inventive system.

FIG. 27 depicts the use of miniaturized and portable device according to the present invention for rapid point-of-care diagnosis of a pathogen, such as COVID-19.

FIG. 28 provides Nyquist plots showing the response of the modified eChip to different concentrations of angiotensin II, the natural substrate of ACE2, ranging from 1 pg mL⁻¹ to 10 μg mL⁻¹

FIG. 29 illustrates the results of an investigation concerning Nafion concentration optimization for a permselective membrane on the present devices.

FIG. 30 shows the results of a study of the effect of sample pretreatment steps on the detection of free SARS-CoV-2 SP

FIG. 31 depicts the results of a kinetic study of the interaction between SARS-CoV-2 SP and DETECT 1.0.

FIGS. 32A and 32B provide calibration curves for free SP in PBS solution (FIG. 32A) and in VTM medium (FIG. 32B).

FIG. 33 depicts an equivalent circuit used for the extraction of the R_{CT} values used in all EIS measurements. R_{S}=electrolyte resistance, R_{CT}=charge transfer resistance, CPE=constant phase element, and W=Warburg component (diffusion-limited mass transport).

FIG. 34 illustrates the relative R_{CT} response extracted from the Nyquist plots for 21 successive EIS measurements of PBS medium using the same biosensor (eChip). The relative standard deviation (RSD) of the R_{CT} values obtained for 21 consecutive measurements was 5.3%, demonstrating an adequate stability for a long operation time (1.5 hours).

FIG. 35 illustrates the results of recording open circuit potential for 60 minutes from an inventive biosensor. During the initial 30 min, the sensor was exposed to a PBS solution, after which it was subjected to 1 ng mL⁻¹ SP for the remaining 30 minutes of the experiment. The biosensor exhibited high stability with an RSD of 0.76% in the potential over the 30 minutes of exposure to SP.

FIG. 36 illustrates the results of a reproducibility test in which normalized sensitivity for 10 different biosensors (10 electrodes from different fabrication batches) was assessed. An analytical curve using free SP in the concentration range of 1 pg mL⁻¹ to 1 ng mL⁻¹ was constructed for each eChip. The relative standard deviation (RSD) value obtained was 6.8%, which represents an adequate reproducibility of the method considering that the functionalization step was not automated.

FIG. 37 depicts the results of an assessment of the stability (shelf-life) of DETECT in different conditions of storage (25° C.-black square, 8° C.—red circles, and −20° C.—blue triangles) over 10 days.

FIG. 38 provides the results of a test involving measurement of samples of SARS-CoV-2 subjected to heat inactivation.

FIG. 39 illustrates how the inventive system was used for detection of SARS-CoV-2 in a prospective cohort study.

FIGS. 40A and 40B provide information concerning a clinical study that was performed in the context of the COVID-19 pandemic in Philadelphia, Pa.

### DETAILED DESCRIPTION OF ILLUSTRATIVE EMBODIMENTS

The presently disclosed inventive subject matter may be understood more readily by reference to the following detailed description taken in connection with the accompanying examples, which form a part of this disclosure. It is to be understood that these inventions are not limited to the specific formulations, methods, articles, or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a detection moiety” is a reference to one or more of such moieties and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” can refer to a value of 7.2 to 8.8, inclusive. This value may include “exactly 8”. In addition, when the term “about” precedes a range, it is intended to modify both the recited lower end and the recited upper end of the range. For example, the phrase “about 1 to 5” means “about 1 to about 5”. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

In the present disclosure, relevant publications are cited in abbreviated format, except in the section, infra, following the heading “References”, in which the full citations of such references are provided.

### I. Detection of SARS-CoV-2 and Other Pathogens

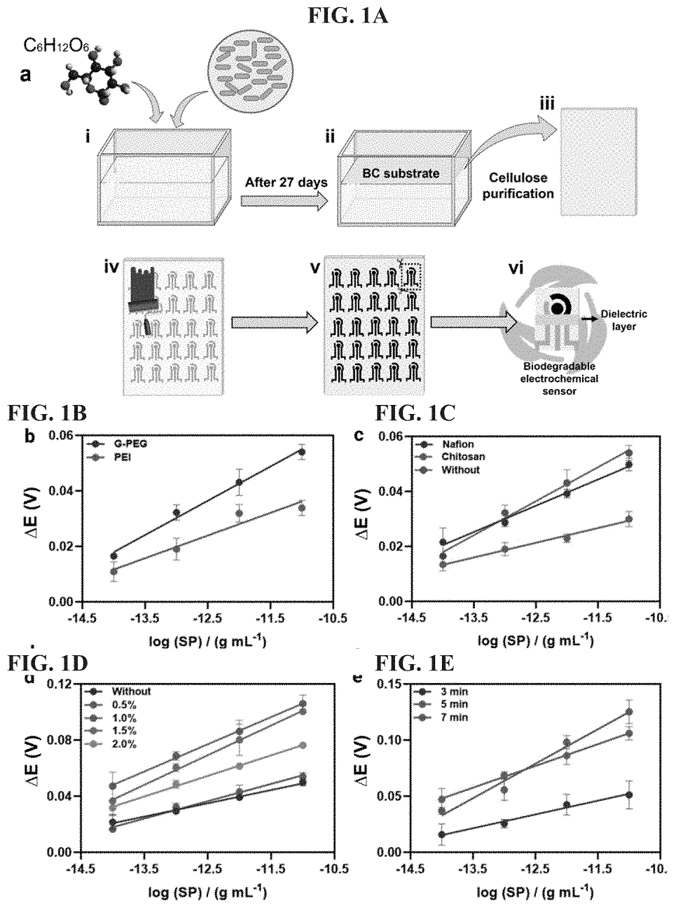

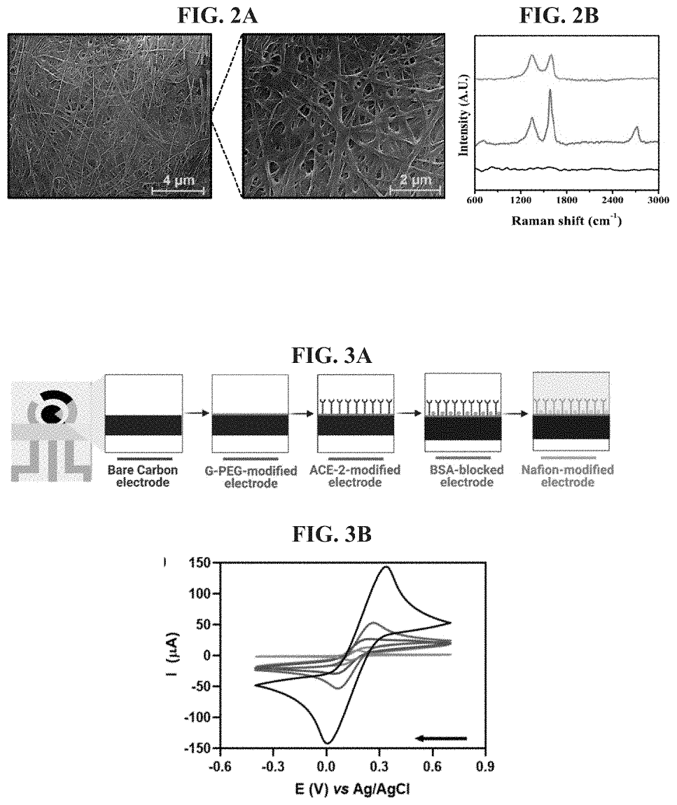

As noted above, there is an urgent need to develop approaches to detect and diagnose both viral and bacterial infections. The present inventors have developed devices that may be cheaply produced and sold, and are capable of diagnosing microbial infections in 10 seconds, representing a vastly cheaper and faster alternative to current state-of-the-art methods used in hospitals (>$100 and diagnosis time of 24 hours) (FIGS. 1 and 2). For example, the devices may be purposed to rapidly detect the virus SARS-CoV-2. The instant technology provides the transformative ability of detecting dangerous infections through its simple design, speed, disposability and ease of operation. The presently disclosed portable electrochemical paper-based devices can use minimal sample volumes, costs less than $3 to produce and can detect pathogens such as SARS-CoV-2 within 10 minutes, and are vastly cheaper and faster than current state-of-the-art diagnostics. Furthermore, the inventive devices accurately and precisely detected 13 emerging SARS-CoV-2 variants and demonstrated exceptional sensitivity, specificity, and accuracy for 65 tested clinical nasopharyngeal/oropharyngeal (NP/OP) samples. The portable and easily operable test device disclosed herein will therefore enable widespread deployment, large-scale testing, and population-level surveillance. Furthermore, to address the issue of environmental harm caused by the disposal of PCBs, the devices utilize a bacterially produced substrate to function, thus providing a rapid, low-cost, and biodegradable diagnostic test for COVID-19 in a form that represents an alternative biodegradable substrate for biosensor development. Thus, the presently disclosed invention has the potential to transform the way we diagnose pathogenic infections, including those that are currently untreatable, thus improving treatment outcome, potentially extending patient survival, and minimizing healthcare costs.

Accordingly, provided herein are devices comprising a substrate that includes a top surface and a back surface; and, an electrode on the top surface of the substrate, wherein the electrode is functionalized with a detection moiety, such as one that binds SARS-CoV-2 spike protein, and a chemical cross linker comprising polyethylene glycol (PEG) that enables immobilization of the detection moiety that binds SARS-CoV-2 spike protein on the electrode.

The substrate may comprise any material that does not interfere with the ability of the electrode to function as intended. For example, the substrate may comprise paper, cardboard, plastic (e.g., polymer), or textile. When the substrate is intended for use as a wearable, it may be of the same material as a traditional bandage, such as plastic or flexible fabric. In order to address the previously described concerns associated with the use of PCB substrates, the present inventors have developed substrate materials that comprise bacterial cellulose (BC). BC is an extracellular polymer synthesized by species of bacteria belonging to several genera: Agrobacterium, Gluconacetobacter, and Sarcina. As a material, BC is nontoxic and low cost and also exhibits several advantages over commercial paper, such as reduced fiber diameter, no use of chemical methods or processes in its manufacture, and high purity. Accordingly, the substrate may comprise bacterial cellulose. In some embodiments, the substrate comprises bacterial nanocellulose.

The electrode may be adhered to the substrate according to any suitable approach, and those of ordinary skill in the art can readily identify numerous approaches for applying an electrode material (e.g., a conductive paste) to a substrate in order to form an electrode. In some embodiments, the electrode is screen-printed onto the top surface of the substrate. In some embodiments, the electrode is wax-printed onto the top surface of the substrate.

The surfaces of the electrode on the substrate may be modified in order to enable binding to the detection moiety. For example, the electrode may be surface-functionalized with thiol groups. Functionalization with thiol groups can be used to form a disulfide bond with a detection moiety. In some embodiments, a disulfide bond occurs between the surface-functionalized electrode and an N-terminal cysteine residue that is engineered onto a detection moiety. For example, the detection moiety that binds SARS-CoV-2 spike protein is human Angiotensin Converting Enzyme 2 (ACE2), an amino acid sequence representing a fragment of ACE2, or an antibody. Any of these detection moieties may be engineered to include an N-terminal cysteine residue that can form a disulfide bond with thiol groups on the electrode in order to securely attach the detection moiety to the electrode. In some embodiments, a detection moiety may be immobilized on the surface of the electrode by crosslinking the detection moiety, such as by using a chemical cross-linker. For example, the detection moiety may be immobilized on the surface of the electrode by crosslinking the detection moiety using polyethylene glycol (PEG). The PEG be conjugated with graphene oxide, and thereby be used as G-PEG. In certain embodiments, ACE2 or a fragment thereof is immobilized on the electrode via an amide bond between the G-PEG and the N-terminus of ACE2 or the fragment thereof. Full-length ACE2 can be recombinantly generated in E. coli using previously established methods (Chan et al., 2020). A peptide of representing a fragment of ACE2 can alternatively be synthesized chemically. In some embodiments, the detection moiety is ACE2, and the ACE2 is applied onto the electrode such that the resulting amount of ACE2 on the electrode is 2.68 μg.

The present inventors have developed an electrochemical analytical device for detecting infections in real time. Impedimetric measurements by electrochemical impedance spectroscopy (EIS) provide qualitative and quantitative data for diagnosing COVID-19 directly from biological samples, such as human blood serum or saliva, through the precise detection of changes in charge transfer resistance due to the detection moiety-virus interaction. For the presently disclosed devices, electrochemical impedance spectroscopy measurements can be used to detect the selective binding of SARS-CoV-2 with the detection moiety, such as ACE2, which interacts specifically with the spike protein of SARS-COV-2, or a peptide representing a fragment of ACE2 that interacts directly with SARS-CoV-2 (FIG. 4). As disclosed herein, electrochemical impedance spectroscopy readings indicate differences in resistance after application of a steady potential and a range of frequency. The specificity of the interactions between ACE2 or peptides and the viral spike protein allow detection of the SARS-CoV-2 in a sample. In some embodiments, portable screen-printed carbon electrodes are chemically functionalized by anchoring the detection moiety to the electrode surface. As described above, functionalization can be achieved through chemical deposition and formation of disulfide bonds between an N-terminal cysteine residue that will be engineered into both ACE2 and the peptide, and the thiol-functionalized electrode surface. The present inventors have previously engineered numerous peptides with an added cysteine for functionalization purposes.

Blocking agents, such as ethanolamine and bovine serum albumin, may be used to cover the remaining exposed surface of the electrode to avoid unspecific interactions and biofouling of the transductor surface, providing sensitive and selective SARS-COV-2 recognition. Thus, the present devices may comprise a blocking layer over the electrode.

The surface of the electrode can also or alternatively be functionalized by forming a membrane that is protective, permselective, or both in order to enhance the robustness of the analytical device. The phrase “on the electrode” with reference to the membrane can refer to a condition in which the membrane is in direct contact with the electrode, or to a condition in which there are intervening structures between the membrane and the electrode. For example, there may be a blocking layer between the membrane and the electrode, and in such a situation, the membrane may still be referred to as being “on the electrode”, albeit in an indirect fashion. The membrane may be formed from a polymeric material. In some embodiments, the protective membrane can be formed by applying a solution that contains Nafion to the surface of the electrode. The Nafion solution can contain, for example, about 0.1% to about 5.0% m/v Nafion. In some embodiments, the Nafion solution contains about 0.5% to about 3% m/v Nafion. In certain embodiments, the Nafion solution contains about 0.5% to about 2% m/v Nafion. In some embodiments, the solution contains 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5% m/v Nafion.

The EIS may be recorded using the Squidstat Plus (Admiral Instruments) analyzer at open circuit potential and a frequency range from 10⁵ to 10⁻² Hz using an alternated current signal of 10 mV amplitude. The changes in resistance to charge transfer (R_{CT}), before and after exposure of the biosensor to contaminated biofluids (e.g., human blood serum and saliva samples), can used to provide qualitative and quantitative results for COVID-19 diagnosis. The R_{CT} response will increase due to the binding between ACE2-SARS-CoV-2 or peptide-SARS-CoV-2 and this response can used to calibrate the dose-response between the virus and the detection moiety.

Accordingly, the presently disclosed devices may be configured to generate a signal that can be assessed via electrochemical impedance spectroscopy (EIS) when a current is run through the electrode. The device may be configured to generate a signal when the detection moiety is bound to SARS-CoV-2 spike protein that is different from the signal that the device generates when the detection moiety is not bound to SARS-CoV-2 spike protein.

In some embodiments, the device is configured to accept a current that is generated by a potentiostat, and to generate a signal from the current that can be detected by the potentiostat. The potentiostat may be an external component, such as of the conventionally used device. However, in some embodiments, the present devices include a miniaturized potentiostat that can perform at least the essential functions of a traditional, external potentiostat, including generating and delivering a current to the electrode, and detecting the signal produced by the device when a current is run through the electrode.

In some embodiments, the present devices can be used to detect SARS-CoV-2 on cell phones through the use of an app and a miniaturized potentiostat.

The device may be wearable, and as such may include an adhesive on the back face of the substrate that is compatible with a subject's skin.

The present devices retain a favorable degree of stability following storage. For example, the devices may retain about 50% of their original sensitivity following storage at 8° C. for 48 hours. In some embodiments, the devices may retain more than 50% of their original sensitivity following storage at −20° C. up to about 10 days. The devices may also retain about 50% of their original sensitivity following storage at −20° C. for about 10 days.

The devices according to the present disclosure are extremely sensitive relative to prior devices for the detection of pathogens. In some embodiments, the limit of detection of the present devices is about 4-10×10⁻¹⁸ of pathogen per mL of a biological sample containing the pathogen. For example, the limit of detection of the present devices may be about 10, 9.5, 9, 8.5, 8, 7.5, 7, 6.5, 6, 5.5, 5, 4.5, or 4×10⁻¹⁸ of pathogen per mL of a biological sample containing the pathogen. In some embodiments, the limit of detection of SARS-CoV-2 of the present devices is about 4-10×10⁻¹⁸ of SARS-CoV-2 spike protein per mL of a biological sample containing the pathogen. For example, the limit of detection of the present devices may be about 10, 9.5, 9, 8.5, 8, 7.5, 7, 6.5, 6, 5.5, 5, 4.5, or 4×10⁻¹⁸ of SARS-CoV-2 spike protein per mL of a biological sample containing SARS-CoV-2. In one embodiment, the limit of detection of SARS-CoV-2 of the present devices is about 4.3×10⁻¹⁸ of SARS-CoV-2 spike protein per mL of a biological sample containing SARS-CoV-2.

Also provided are wearable articles comprising a device according to any of the embodiments described herein. The article may be, for example, a self-adhesive bandage, a band for wrapping around an appendage of a subject (including an upper or lower arm, a calf, or a forearm, for example), a glove, or a mask. When in the form of a mask, the article may incorporate a device according to the present disclosure at a location that will contact droplets that are expelled from a subject's mouth or nose during breathing, sneezing, or coughing. The article may include a colorimetric functionality that displays a certain color or that changes color when the device detects the presence of SARS-CoV-2.

The present disclosure also pertains to methods for assessing the presence of a pathogen, such as SARS-CoV-2, in a biological sample comprising contacting a device according to the present disclosure with the biological sample; exposing the device to an electrical current in order to generate a signal from the device; and, assessing the signal that is generated by the device electrochemical impedance spectroscopy (EIS) in order to determine the absence or presence of the pathogen in the biological sample. In certain embodiments, the electrical current is an alternating current (AC). The alternating current may have an amplitude of about 5 to about 15 mV. For example, the alternating current may have an amplitude of about 5, 6, 7, 8, 9, 10, 11, 12, 13, 14, or 15 mV. In a specific embodiment, the alternating current has an amplitude of about 10 mV.

### II. Detection of HSV

As noted above, despite the prevalence of HSV-2 infections, there have been no rapid tests available to detect this infectious agent. The present inventors have developed impedimetric biosensors for the rapid, ultrasensitive detection of HSV-2 (FIG. 9A). Instead of traditional genosensors and serological tests, provided herein, for the first time, is the use of a cellular receptor for the development of a novel and accurate electrochemical diagnostic for HSV-2. In some embodiments, inventive technology uses electrodes functionalized with one or more of the conductive polymer polyethyleneimine (PEI), the bioreceptor nectin-1, and a chitosan semipermeable membrane (FIG. 9B). In order to develop a sensitive and robust rapid test, the inventors have developed an optimal strategy to biofunctionalize the working electrode. Under optimal conditions, the present devices can detect the virus within minutes (sample incubation+analysis), displays a very low limit of detection (LOD) of plaque-forming units (PFU) mL-1, and presents high sensitivity, 100% specificity, and very high accuracy.

Accordingly, provided herein are devices comprising a substrate that includes a top surface and a back surface; and, an electrode on the top surface of the substrate, wherein the electrode is functionalized with a detection moiety that binds HSV glycoprotein gD2.

The substrate may comprise any material that does not interfere with the ability of the electrode to function as intended. For example, the substrate may comprise paper, cardboard, plastic (e.g., polymer), or textile. When the substrate is intended for use as a wearable, it may be of the same material as a traditional bandage, such as plastic or flexible fabric.

The electrode may be adhered to the substrate according to any suitable approach, and those of ordinary skill in the art can readily identify numerous approaches for applying an electrode material (e.g., a conductive paste) to a substrate in order to form an electrode. In some embodiments, the electrode is screen-printed onto the top surface of the substrate. In some embodiments, the electrode is wax-printed onto the top surface of the substrate.

The surfaces of the electrode on the substrate may be modified in order to enable binding to the detection moiety. For example, the electrode may be surface-functionalized with thiol groups. Functionalization with thiol groups can be used to form a disulfide bond with a detection moiety. In some embodiments, a disulfide bond occurs between the surface-functionalized electrode and an N-terminal cysteine residue that is engineered onto a detection moiety. For example, the detection moiety that binds HSV glycoprotein gD2 is nectin-1 or an antibody. Any of the detection moieties may be engineered to include an N-terminal cysteine residue that can form a disulfide bond with thiol groups on the electrode in order to securely attach the detection moiety to the electrode. In some embodiments, a detection moiety may be immobilized on the surface of the electrode by crosslinking the detection moiety, such as by using a chemical cross-linker. For example, the detection moiety may be immobilized on the surface of the electrode by crosslinking the detection moiety using polyethylenimine (PEI). In certain embodiments, nectin-1 is immobilized on the electrode via an amide bond between the PEI and a carboxyl group on nectin-1. For example, carboxyl groups on nectin-1, when exposed to EDC-NHS, may be activated to form a stable ester, which undergoes a nucleophilic addition with amino groups on the PEI-modified electrode, such that a stable amide bond is formed between the PEI-modified carbon electrode and nectin-1. Human herpes virus entry mediator (HveC), also called human nectin-1 (residues 31-346), can be recombinantly produced, for example, by baculoviruses. Their purification from infected insect cells was described previously.

The present inventors have developed an electrochemical analytical device for detecting infections by HSV in real time. Impedimetric measurements by electrochemical impedance spectroscopy (EIS) provide qualitative and quantitative data for diagnosing HSV directly from biological samples, such as human blood serum or saliva, through the precise detection of changes in charge transfer resistance due to the detection moiety-virus interaction. For the presently disclosed devices, electrochemical impedance spectroscopy measurements can be used to detect the selective binding of HSV with the detection moiety, such as nectin-1, which interacts specifically with the glycoprotein gD2 of HSV. As disclosed herein, electrochemical impedance spectroscopy readings indicate differences in resistance after application of a steady potential and a range of frequency. The specificity of the interactions between nectin-1 and the glycoprotein gD2 allow detection of the HSV in a sample. In some embodiments, portable screen-printed carbon electrodes are chemically functionalized by anchoring the detection moiety to the electrode surface. As described above, functionalization can be achieved through chemical deposition and formation of disulfide bonds between an N-terminal cysteine residue, and the functionalized electrode surface.

Blocking agents, such as ethanolamine and bovine serum albumin, may be used to cover the remaining exposed surface of the electrode to avoid unspecific interactions and biofouling of the transductor surface, providing sensitive and selective HSV recognition. Thus, the present devices may comprise a blocking layer over the electrode.

The surface of the electrode can also or alternatively be functionalized by forming a membrane that is protective, permselective, or both in order to enhance the robustness of the analytical device. The phrase “on the electrode” with reference to the membrane can refer to a condition in which the membrane is in direct contact with the electrode, or to a condition in which there are intervening structures between the membrane and the electrode. For example, there may be a blocking layer between the membrane and the electrode, and in such a situation, the membrane may still be referred to as being “on the electrode”, albeit in an indirect fashion. The membrane may be formed from a polymeric material. In some embodiments, the protective membrane can be formed by applying a solution that contains Nafion to the surface of the electrode. In other embodiments, the protective membrane can be formed by applying a solution that contains chitosan to the surface of the electrode. The solution can contain, for example, about 0.05% to about 5.0% m/v of the membrane material, e.g., of chitosan. In some embodiments, the solution contains about 0.075% to about 3% m/v, about 0.1% to about 2% m/v, about 0.1% to about 1% m/v, or about 0.25% to about 0.75% m/v of the membrane material, e.g., of chitosan. In some embodiments, the solution contains 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5% m/v of the membrane material, e.g., of chitosan.

The EIS may be recorded using the Squidstat Plus (Admiral Instruments) analyzer at open circuit potential and a frequency range from 10⁵ to 10⁻² Hz using an alternated current signal of 10 mV amplitude. The changes in resistance to charge transfer (R_{CT}), before and after exposure of the biosensor to contaminated biofluids (e.g., human blood serum and saliva samples), can used to provide qualitative and quantitative results for HSV diagnosis. The R_{CT} response will increase due to the binding between the detection moiety (e.g., nectin-1) and HSV glycoprotein gD2 and this response can used to calibrate the dose-response between the virus and the detection moiety.

Accordingly, the presently disclosed devices may be configured to generate a signal that can be assessed via electrochemical impedance spectroscopy (EIS) when a current is run through the electrode. The device may be configured to generate a signal when the detection moiety is bound to glycoprotein gD2 that is different from the signal that the device generates when the detection moiety is not bound to glycoprotein gD2.

In some embodiments, the device is configured to accept a current that is generated by a potentiostat, and to generate a signal from the current that can be detected by the potentiostat. The potentiostat may be an external component, such as of the conventionally used device. However, in some embodiments, the present devices include a miniaturized potentiostat that can perform at least the essential functions of a traditional, external potentiostat, including generating and delivering a current to the electrode, and detecting the signal produced by the device when a current is run through the electrode.

In some embodiments, the present devices can be used to detect HSV on cell phones through the use of an app and a miniaturized potentiostat.

The device may be wearable, and as such may include an adhesive on the back face of the substrate that is compatible with a subject's skin.

The present devices retain a favorable degree of stability following storage. For example, the devices may retain at least 60, 70, 80, or 90% of their original sensitivity following storage at 4° C. for 48 hours. In some embodiments, the devices may retain at least 60, 70, 80% of their original sensitivity following storage at 4° C. for 120 hours. In some embodiments, the devices may retain more than 50% of their original sensitivity following storage at −20° C. up to about 5 days. The devices may also retain about 50% of their original sensitivity following storage at −20° C. for about 7 days.

The devices according to the present disclosure are extremely sensitive relative to prior devices for the detection of pathogens. In some embodiments, the limit of detection of the present devices is about 0.055-0.210 PFU of pathogen per mL of a biological sample containing the pathogen. For example, the limit of detection of the present devices may be about 0.055, 0.06, 0.065, 0.07, 0.075, 0.08, 0.085, 0.09, 0.095, 0.1, 0.11, 0.12, 0.13, 0.14 0.15, 0.16, 0.17, 0.18, 0.19, 0.20, or 0.21 of pathogen per mL of a biological sample containing the pathogen. In some embodiments, the limit of detection of HSV of the present devices is about 0.015-0.09 fg of glycoprotein gD2 per mL of a biological sample containing the HSV. For example, the limit of detection of the present devices may be about 0.015, 0.016, 0.017, 0.018, 0.019, 0.02, 0.022, 0.024, 0.026, 0.028 0.03, 0.032, 0.034, 0.036, 0.038, 0.04, 0.042, 0.044, 0.046, 0.048, 0.05, 0.052, 0.054, 0.056, 0.058, 0.06, 0.062, 0.064, 0.066, 0.068, 0.07, 0.072, 0.074, 0.076, 0.078, 0.08, 0.082, 0.084, 0.086, 0.088, or 0.09 fg of glycoprotein gD2 per mL of a biological sample containing HSV.

Also provided are wearable articles comprising a device according to any of the embodiments described herein. The article may be, for example, a self-adhesive bandage, a band for wrapping around an appendage of a subject (including an upper or lower arm, a calf, or a forearm, for example), a glove, or a mask. When in the form of a mask, the article may incorporate a device according to the present disclosure at a location that will contact droplets that are expelled from a subject's mouth or nose during breathing, sneezing, or coughing. The article may include a colorimetric functionality that displays a certain color or that changes color when the device detects the presence of HSV.

The present disclosure also pertains to methods for assessing the presence of a pathogen, such as HSV, in a biological sample comprising contacting a device according to the present disclosure with the biological sample; exposing the device to an electrical current in order to generate a signal from the device; and, assessing the signal that is generated by the device electrochemical impedance spectroscopy (EIS) in order to determine the absence or presence of the pathogen in the biological sample. In certain embodiments, the electrical current is an alternating current (AC). The alternating current may have an amplitude of about 5 to about 15 mV. For example, the alternating current may have an amplitude of about 5, 6, 7, 8, 9, 10, 11, 12, 13, 14, or 15 mV. In a specific embodiment, the alternating current has an amplitude of about 10 mV.

### III. Further Biosensors for Detection of SARS-CoV-2

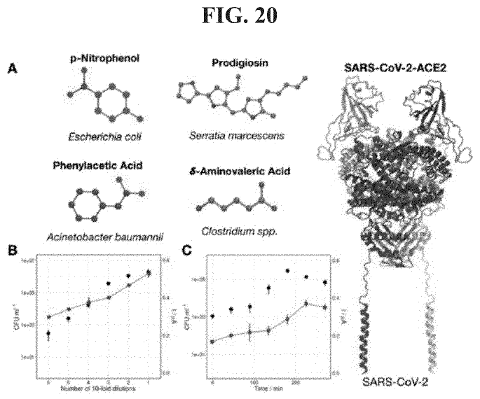

As noted above, there is an urgent need to develop approaches to detect and diagnose both viral and bacterial infections. The present inventors have developed devices that may be cheaply produced and sold, and are capable of diagnosing microbial infections in 10 seconds, representing a vastly cheaper and faster alternative to current state-of-the-art methods used in hospitals (>$100 and diagnosis time of 24 hours) (FIGS. 19 and 20). For example, the devices may be purposed to rapidly detect the virus SARS-CoV-2. The instant technology provides the transformative ability of detecting dangerous infections through its simple design, speed, disposability and ease of operation. The presently disclosed portable electrochemical paper-based devices can use minimal sample volumes (10 μL), costs less than $1 to produce and can detect pathogens such as SARS-CoV-2 within 10 minutes, and are vastly cheaper and faster than current state-of-the-art diagnostics. The portable and easily operable test device disclosed herein will enable widespread deployment, large-scale testing, and population-level surveillance. Thus, the presently disclosed invention has the potential to transform the way we diagnose pathogenic infections, including those that are currently untreatable, thus improving treatment outcome, potentially extending patient survival, and minimizing healthcare costs.

Accordingly, provided herein are devices for assessing the presence of a pathogen, such as SARS-CoV-2, in a biological sample, the devices comprising a substrate comprising a top surface and a back surface; and, an electrode on the top surface of the substrate, wherein the electrode is functionalized with a detection moiety, such as one that binds SARS-CoV-2 spike protein.

The substrate may comprise any material that does not interfere with the ability of the electrode to function as intended. For example, the substrate may comprise paper, cardboard, plastic (e.g., polymer), or textile. When the substrate is intended for use as a wearable, it may be of the same material as a traditional bandage, such as plastic or flexible fabric.

The electrode may be adhered to the substrate according to any suitable approach, and those of ordinary skill in the art can readily identify numerous approaches for applying an electrode material (e.g., a conductive paste) to a substrate in order to form an electrode. In some embodiments, the electrode is screen-printed onto the top surface of the substrate. In some embodiments, the electrode is wax-printed onto the top surface of the substrate.

The surfaces of the electrode on the substrate may be modified in order to enable binding to the detection moiety. For example, the electrode may be surface-functionalized with thiol groups. Functionalization with thiol groups can be used to form a disulfide bond with a detection moiety. In some embodiments, a disulfide bond occurs between the surface-functionalized electrode and an N-terminal cysteine residue that is engineered onto a detection moiety. For example, the detection moiety that binds SARS-CoV-2 spike protein is human Angiotensin Converting Enzyme 2 (ACE2), the amino acid sequence IEEQAKTFLDKFNHEAEDLFYQS (SEQ ID NO:1), or an antibody. Any of these detection moieties may be engineered to include an N-terminal cysteine residue that can form a disulfide bond with thiol groups on the electrode in order to securely attach the detection moiety to the electrode. In some embodiments, a detection moiety may be immobilized on the surface of the electrode by crosslinking the detection moiety, such as by using a chemical cross-linker. For example, the detection moiety may be immobilized on the surface of the electrode by crosslinking the detection moiety using the bifunctional chemical cross-linker glutaraldehyde (GA). In certain embodiments, ACE2 or SEQ ID NO:1 is immobilized on the electrode via an amide bond between the glutaraldehyde and the N-terminus of ACE2 or SEQ ID NO:1. Full-length ACE2 and the 23-mer peptide of SEQ ID NO: 1 can be recombinantly generated in E. coli using previously established methods (Chan et al., 2020). The peptide of SEQ ID NO: 1 can alternatively be synthesized chemically. In some embodiments, the detection moiety is ACE2, and the ACE2 is applied onto the electrode such that the resulting amount of ACE2 on the electrode is 2.68 μg.

The present inventors have developed an electrochemical analytical device for detecting infections in real time. FIG. 19A depicts a down-side photograph of a device coupled to an adhesive wearable for detecting Pyo through an electrochemical redox process, as provided in FIG. 19B. FIG. 19C shows the effect of pH on the electrochemical behavior of Pyo. FIG. 19D provides an EP vs. pH plot, and FIG. 19E provides square wave voltammograms for successive additions of Pyo with concentrations ranging from 50 to 1000 nmol/L. The inset provides an analytical curve constructed with the peak current for both electrochemical processes.

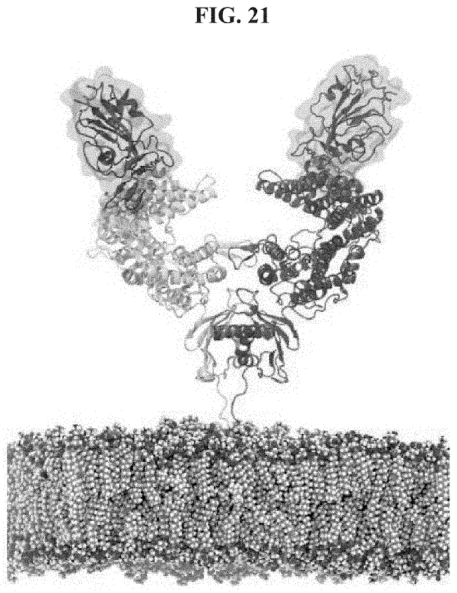

The potentially wearable device detects, through cyclic voltammetry, redox-active metabolites uniquely produced by pathogenic infectious agents. In FIG. 20A, redox bacterial biomarkers (left) and ACE2 protein (right) were detectable by the device. On the right, the SARS-CoV-2-ACE2 structure is depicted. ACE2 and peptides derived from its structure are detectable by the present devices. FIG. 21 depicts the results of molecular dynamics simulations performed by the inventors of the region of the SARS-CoV-2 viral spike protein (blue) that binds to the human ACE2 protein (red and yellow). FIG. 20B provides Pseudomonas aeruginosa CFU/mL counts of overnight culture dilution compared to current measured at pH2. FIG. 20C shows bacterial growth over time in LB medium determined by the device in relation to CFU/mL counts.

Impedimetric measurements by electrochemical impedance spectroscopy (EIS) provide qualitative and quantitative data for diagnosing COVID-19 directly from biological samples, such as human blood serum or saliva, through the precise detection of changes in charge transfer resistance due to the detection moiety-virus interaction.

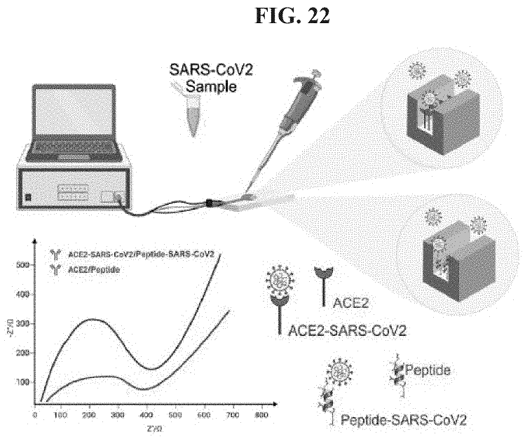

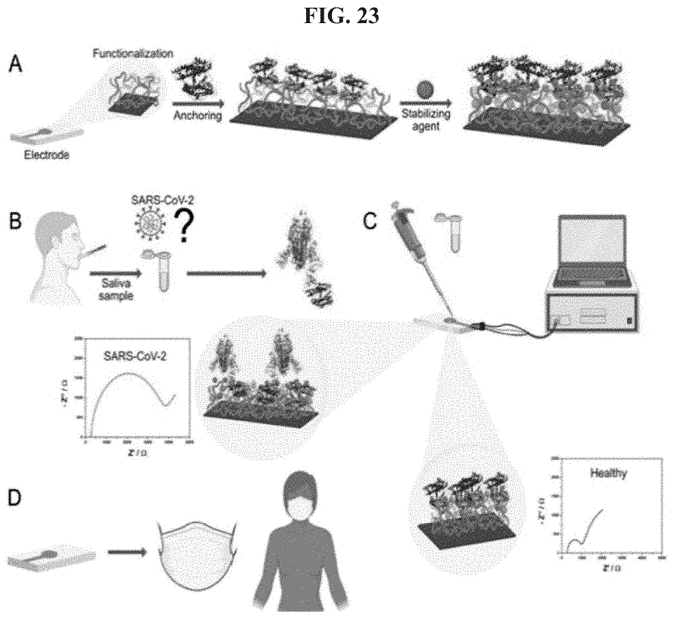

Thus, electrochemical impedance spectroscopy measurements can be used to detect the selective binding of SARS-CoV-2 with the detection moiety, such as ACE2, which interacts specifically with the spike protein of SARS-COV-2, or a SEQ ID NO:1, which represents a 23-mer peptide that interacts directly with SARS-CoV-2 (FIG. 22). As provided in FIG. 22, electrochemical impedance spectroscopy readings indicate differences in resistance after application of a steady potential and a range of frequency. The specificity of the interactions between ACE2 or peptides and the viral spike protein allow detection of the SARS-CoV-2 in a sample. In some embodiments, portable screen-printed carbon electrodes are chemically functionalized by anchoring the detection moiety to the electrode surface. As described above, functionalization can be achieved through chemical deposition and formation of disulfide bonds between an N-terminal cysteine residue that will be engineered into both ACE2 and the 23-mer peptide, and the thiol-functionalized electrode surface. The present inventors have previously engineered numerous peptides with an added cysteine for functionalization purposes.

Blocking agents, such as ethanolamine and bovine serum albumin, may be used to cover the remaining exposed surface of the electrode to avoid unspecific interactions and biofouling of the transductor surface, providing sensitive and selective SARS-COV-2 recognition. Thus, the present devices may comprise a blocking layer over the electrode.

The surface of the electrode can also or alternatively be functionalized by forming a membrane that is protective, permselective, or both in order to enhance the robustness of the analytical device. The phrase “on the electrode” with reference to the membrane can refer to a condition in which the membrane is in direct contact with the electrode, or to a condition in which there are intervening structures between the membrane and the electrode. For example, there may be a blocking layer between the membrane and the electrode, and in such a situation, the membrane may still be referred to as being “on the electrode”, albeit in an indirect fashion. The membrane may be formed from a polymeric material. In some embodiments, the protective membrane can be formed by applying a solution that contains Nafion to the surface of the electrode. The Nafion solution can contain, for example, about 0.1% to about 5.0% v/v Nafion. In some embodiments, the Nafion solution contains about 0.5% to about 3% v/v Nafion. In some embodiments, the solution contains 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5% v/v Nafion.

The EIS may be recorded using the Squidstat Plus (Admiral Instruments) analyzer at open circuit potential and a frequency range from 10⁵ to 10⁻² Hz using an alternated current signal of 10 mV amplitude. The changes in resistance to charge transfer (R_{CT}), before and after exposure of the biosensor to contaminated biofluids (e.g., human blood serum and saliva samples), can used to provide qualitative and quantitative results for COVID-19 diagnosis. The R_{CT} response will increase due to the binding between ACE2-SARS-CoV-2 or peptide-SARS-CoV-2 and this response can used to calibrate the dose-response between the virus and the detection moiety (FIG. 22).

Accordingly, the presently disclosed devices may be configured to generate a signal that can be assessed via electrochemical impedance spectroscopy (EIS) when a current is run through the electrode. The device may be configured to generate a signal when the detection moiety is bound to SARS-CoV-2 spike protein that is different from the signal that the device generates when the detection moiety is not bound to SARS-CoV-2 spike protein.

In some embodiments, the device is configured to accept a current that is generated by a potentiostat, and to generate a signal from the current that can be detected by the potentiostat. The potentiostat may be an external component, such as of the conventionally used device. However, in some embodiments, the present devices include a miniaturized potentiostat that can perform at least the essential functions of a traditional, external potentiostat, including generating and delivering a current to the electrode, and detecting the signal produced by the device when a current is run through the electrode.

In some embodiments, the present devices can be used to detect SARS-CoV-2 on cell phones through the use of an app and a miniaturized potentiostat.

The device may be wearable, and as such may include an adhesive on the back face of the substrate that is compatible with a subject's skin.

The present devices retain a favorable degree of stability following storage. For example, the devices may retain about 50% of their original sensitivity following storage at 8° C. for 48 hours. In some embodiments, the devices may retain more than 50% of their original sensitivity following storage at −20° C. up to about 10 days. The devices may also retain about 50% of their original sensitivity following storage at −20° C. for about 10 days.

The devices according to the present disclosure are extremely sensitive relative to prior devices for the detection of pathogens. In some embodiments, the limit of detection of the present devices is about 3-10 PFU of pathogen per mL of a biological sample containing the pathogen. For example, the limit of detection of the present devices may be about 10, 9, 8, 7, 6, 5, 4, or 3 PFU of pathogen per mL of a biological sample containing the pathogen. In some embodiments, the limit of detection of SARS-CoV-2 of the present devices is about 3-10 fg of SARS-CoV-2 spike protein per mL of a biological sample containing the pathogen. For example, the limit of detection of the present devices may be about 10, 9, 8, 7, 6, 5, 4, or 3 fg of SARS-CoV-2 spike protein per mL of a biological sample containing SARS-CoV-2. In one embodiment, the limit of detection of SARS-CoV-2 of the present devices is about 2.8 fg of SARS-CoV-2 spike protein per mL of a biological sample containing SARS-CoV-2.

Also provided are wearable articles comprising a device according to any of the embodiments described herein. The article is may be, for example, a self-adhesive bandage, a band for wrapping around an appendage of a subject (including an upper or lower arm, a calf, or a forearm, for example), a glove, or a mask. When in the form of a mask, the article may incorporate a device according to the present disclosure at a location that will contact droplets that are expelled from a subject's mouth or nose during breathing, sneezing, or coughing. The article may include a colorimetric functionality that displays a certain color or that changes color when the device detects the presence of SARS-CoV-2.

The present disclosure also pertains to methods for assessing the presence of a pathogen, such as SARS-CoV-2, in a biological sample comprising contacting a device according to the present disclosure with the biological sample; exposing the device to an electrical current in order to generate a signal from the device; and, assessing the signal that is generated by the device electrochemical impedance spectroscopy (EIS) in order to determine the absence or presence of the pathogen in the biological sample. In certain embodiments, the electrical current is an alternating current (AC). The alternating current may have an amplitude of about 5 to about 15 mV. For example, the alternating current may have an amplitude of about 5, 6, 7, 8, 9, 10, 11, 12, 13, 14, or 15 mV. In a specific embodiment, the alternating current has an amplitude of about 10 mV.

### EXAMPLES

The present invention is further defined in the following Examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1—Fabrication of SARS CoV-2 Biosensor

In this study, for the sensitive diagnosis of SARS-CoV-2 directly at the point of need, the focus was on an approach that does not require sophisticated instrumentation, using instead a low-cost and portable potentiometer that detects the difference in electrical potential between a stable reference electrode (RE) and the functional working electrode (WE) fabricated on a flexible bacterial cellulose (BC) substrate. The WE is selective for the target analyte, which causes a charge change at its surface upon target species recognition, eliminating the need for a redox probe for analysis.

BC is typically a pure mat of nanosized cellulose fibers. Briefly, for BC production, Gluconacetobacter hansenii was incubated in Hestrin-Schramm (HS) medium with 20 g L⁻¹ glucose. After 27 days, a BC material was collected and treated with 5 mmol L⁻¹ NaOH at 80° C., which was subsequently washed with deionized water abundantly and, after drying, resulted in a clear sheet. The BC substrate was used as a platform for the screen-printing of the electrochemical systems, which were cut to 2.5×2.0 cm dimensions (FIG. 1A). FIG. 1A illustrates the fabrication steps of the biodegradable BC substrate and the electrochemical devices. First, the bacterium Gluconacetobacter hansenii was incubated in HS medium with 20 g L−1 glucose (i); after 27 days, a BC substrate was collected and treated with NaOH 5 mmol L−1 at 80° C. (ii), resulting in a clear sheet (iii). Next, the biodegradable BC substrate was screen-printed with carbon and Ag/AgCl conductive ink (iv), resulting in a device with 3 electrodes (WE, CE, and RE), which were cut out using a scissor (v), yielding a portable, biodegradable, and inexpensive electrochemical sensor (vi).

To obtain a selective and sensitive biosensor, an evaluation was performed regarding the best of two approaches to anchoring the ACE2 receptor on the carbon screen-printed electrodes. First, the WE was modified with amine-functionalized G-PEG. Second, the WE was modified with the conducting polymer PEI, which also contains NH₂-functional groups (25, 26). G-PEG provided significant discrimination of the analytical signal at the low concentrations of SP analyzed (10⁻¹⁴-10⁻¹¹ g mL⁻¹) (FIG. 1B, showing modification of the WE to anchor ACE2 using 2 mg mL⁻¹ G-PEG (black circles) or 1 mg mL⁻¹ PEI (red circles)), probably because the large surface area of the nanomaterial provided more bioconjugation sites, facilitating the interactions between ACE2 and SP (27). Thus, the G-PEG modification strategy was used throughout this study.

The fabrication, modification, and functionalization steps were then optimized to obtain a more robust and sensitive biosensor for SARS-CoV-2 SP detection. The WE was modified with G-PEG using the drop-casting method and incubating for 60 min at 37° C. to dry. This procedure introduces amine groups on the WE surface for bioconjugation. Next, the ACE2 receptor containing EDAC (1-ethyl-3-(−3-dimethylaminopropyl) carbodiimide)+NHS was dropped on the WE modified with G-PEG and kept for 30 min at 37° C. When the carboxyl groups of ACE2 were exposed to EDAC-NHS, they were activated to form a stable ester, which undergoes a nucleophilic addition with the amino groups present on the WE, resulting in a stable amide bond between the carbon WE/G-PEG and ACE2 (25, 28). The remaining unmodified sites of the WE surface were then blocked using a 1.0% (m/v) BSA solution.

Polymeric membranes can protect the electrode surface against biofouling when this surface is exposed to the sample's complex matrix, and can also provide superficial preconcentration of chemical species. For this study, analytical curves were made at concentrations ranging from 1×10⁻¹⁴ to 1×10⁻¹¹ g mL⁻¹ SP in 0.1 mol L⁻¹ phosphate buffer solution (PBS) (pH=7.4) to compare 3 strategies of biosensor modification: (1) using 0.5% Nafion®; (2) using 0.5% chitosan; and (3) without any permeable membrane. The Nafion® layer resulted in the highest sensitivity of the biosensor (FIG. 1C, showing performance of the electrochemical biosensor modified with 0.5% (m/v) Nafion® (black circles), modified with 0.5% (m/v) chitosan (red circles), and without membrane layer (blue circles)), presumably because its anionic membrane allows small positively charged species to cross the biosensing surface and become preconcentrated close to this surface (30).

Given the results presented in FIG. 1C, a study was performed regarding the effects of changing the proportion of Nafion® on the modified biosensor since this proportion would directly impact membrane thickness. FIG. 1D (effect of Nafion® concentration on the sensitivity of the method: 0.0% (black circles); 0.5% (m/v; red circles); 1.0% (m/v; blue circles); 1.5% (m/v; purple circles); and 1.0% (m/v; green circles)) shows the performance of the biosensor at various Nafion® concentrations; 1.0% (m/v) Nafion® provided the highest detectability and analytical sensitivity. Thus, this condition was selected for further studies.

An evaluation was performed concerning time of incubation of SP with the surface of the modified biosensor would yield the best analytical performance for SARS-CoV-2 detection. The experiment was carried out in triplicate with an interval concentration ranging from 10⁻¹⁴ to 10⁻¹¹ g mL⁻¹ of SARS-CoV-2 SP (FIG. 1E). The results revealed that 5 min and 7 min of incubation time provided similar detectability. However, this optimization was based on the analytical sensitivity (slope) parameter obtained by analytical curves. Thus, 7 minutes was chosen as the optimal incubation time, which provided high detectability and sensitivity. These results demonstrate the fast-binding kinetics between SP and the ACE2 receptor immobilized on the electrode surface, highlighting the efficiency of the inventive biosensor architecture.

Materials. All reagents used in the experiments were of analytical grade. Deionized water (resistivity ≥18 MΩ cm at 25° C.) was obtained from a Milli-Q Advantage-0.10 purification system (Millipore). Human ACE2 Fc Chimera was obtained from GenScript. SP was kindly donated by Dr. Scott Hensley from the University of Pennsylvania. Graphene oxide conjugated with polyethylene glycol (G-PEG) amine-functionalized, N-(3-dimethylaminopropyl)-N-ethylcarbodiimide hydrochloride (EDAC), and N-Hydroxysuccinimide (NHS) with a degree of purity ≥98% and phosphate buffer saline solution, pH=7.4, were purchased from Sigma-Aldrich. Carbon and Ag/AgCl conductive inks and a dielectric ink were acquired from Creative Materials.

Fabrication of bacterial cellulose (BC) substrate. BC substrates were produced by G. hansenii (ATCC 53582), schematically illustrated in FIG. 1A. First, the bacteria were inoculated in 1.0 L of Hestrin-Schramm (HS) medium, which had been previously autoclaved at 121° C. for 15 minutes. Then, the mixture was transferred to a plastic container of approximately 40×20 cm and left at room temperature, 25±3° C., for 27 days in static conditions. Subsequently, the BC film formed was collected and cleaned using 0.1 mol L⁻¹ NaOH solution at 80° C. for 4 h. Finally, the pretreated BC was washed with deionized water to remove alkalinity and kept at 80° C. in an incubator until completely dry. This procedure provided a biodegradable substrate with a thickness of 90.0±1.0 μm.

The electrochemical devices were manufactured by the screen-printing method with 3-electrode configuration cells (dimensions: 2.5×2.0 cm) on the biodegradable BC substrate. Carbon conductive ink was used to fabricate the WE and counter electrode (CE), and Ag/AgCl conductive ink was used to fabricate the reference electrode (RE). To cure the conductive tracks, the printed BC substrates were placed in a thermal oven at 70° C. for 30 minutes. After the curing step, the devices were cut into small pieces (2.5×2.0 cm). To delimit the electrode area, a non-conductive ink was used, and the devices were submitted to an additional curing step under the same conditions as described above.

Modification of BC electrodes. To prepare the electrochemical biosensor, 5.0 μL of 2 mg mL⁻¹ G-PEG solution was dropped on the carbon WE and allowed to dry for 60 min at 37° C. Next, 5.0 μL of a mixture of 0.33 mg mL⁻¹ ACE2 receptor containing 25 mmol L⁻¹ EDAC and 50 mmol L⁻¹ of NHS solution were drop-casted on the surface of the WE and incubated at 37° C. for 60 min. The unmodified zones of the WE were blocked with 5.0 μL of 1% (m/v) BSA solution, and the WE was stored for 30 min at 37° C. to dry, to avoid non-specific interactions of other biomolecules present in the sample with the biosensor's surface. Finally, 5.0 μL of 1.0% (m/v) Nafion® was deposited onto the WE, and the WE was incubated at 37° C. for 60 min. The biosensor was then washed with PBS 0.1 mol L⁻¹ (pH=7.4) before use.

### Example 2—Electrochemical Characterization of Biosensor and Detection of SARS CoV-2

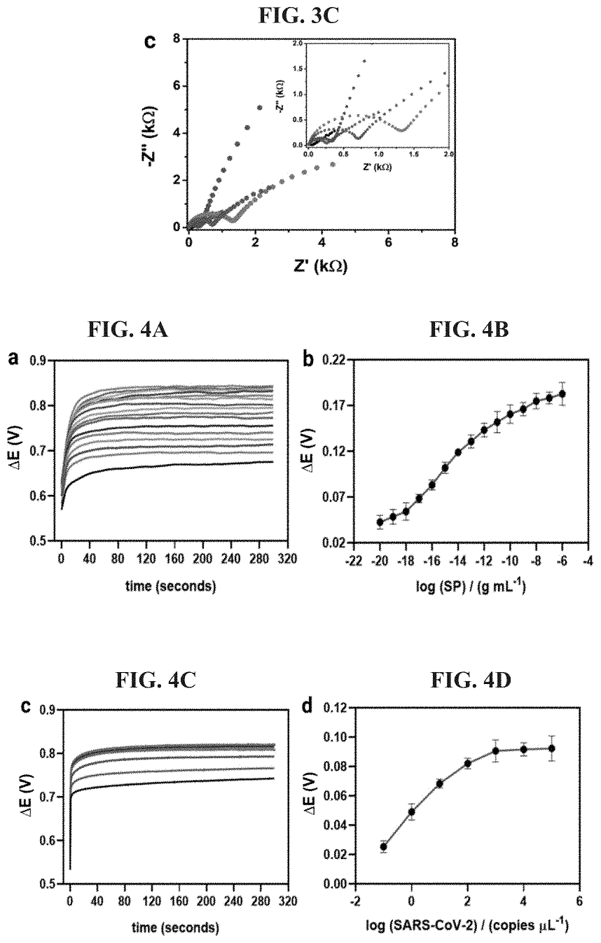

To evaluate the electrochemical behavior of the sensor modification, cyclic voltammetry (CV) and electrochemical impedance spectroscopy (EIS) were used to record measurements of each functionalization step of the biosensor (FIGS. 3A-3C). FIG. 3A provides a schematic representation of stepwise functionalization of the electrochemical biosensor. FIG. 3B shows CVs recorded in all steps of modification of the electrochemical biosensor using 5.0 mmol L⁻¹ [Fe(CN)₆]^{-3/-4} containing 0.1 mol L⁻¹ KCl as supporting electrolyte in a potential window ranging from −0.4 V to 0.7 V at a scan rate of 50 mV s⁻¹. For FIG. 3C, Nyquist plots were obtained in the same experimental conditions as used for FIG. 3B. The inset in FIG. 2C shows a zoomed-in view of the plots at high-frequency regions. Conditions: frequency range from 1×105 Hz to 0.1 Hz and 10 mV amplitude; measurements performed at room temperature. The colors displayed in the CVs and Nyquist plots are related to each step modification illustrated in FIG. 3A.

The bare carbon screen-printed electrode on the biodegradable BC substrate presented a defined redox process with peak currents (ip) of 148.5 μA and resistance to charge transfer (R_{CT}) of 40.2 S2 (FIGS. 3B and 3C, respectively). After modifying the WE with G-PEG, the ip drastically decreased to 51.7 μA and the R_{CT} increased to 312.6Ω. These results were in line with the low electrical conductivity of graphene oxide; PEG contributes to a lower charge transfer. Next, ACE2 was covalently anchored to the WE surface by the EDAC-NHS approach. This step contributed to a decrease in the ip to 27.5 μA and increased the R_{CT} value to 451.3Ω. Subsequently, the remaining nonspecific sites of the electrode were blocked with 0.1% (m/v) bovine serum albumin (BSA), resulting in an ip of 14.0 μA and R_{CT} of 824.7 S2, which is related to the modification with a nonconductive layer on the WE surface. In the last step of biosensor fabrication, the WE surface was modified using a 1% (m/v) Nafion® permeable membrane to enhance the robustness of the biosensor. Hence, the ip decreased to 7.9 μA and the R_{CT} increased to 1,466Ω.

Analytical performance of the biosensor. Highly specific interactions between the SP and the ACE2-coated electrode induce a potential variation. In these interactions, the output voltage is logarithmically correlated to the concentration of the target species in the solution, similarly to traditional ion-selective electrodes (ISE). Thus, when the analyte (SP) is present in the analyzed sample, the binding of the analyte to the functional membrane receptor (ACE2) produces an excess of surface charge on the electrode surface. Consequently, a potential change develops at the electrode, which can be used for diagnostic purposes. The presently disclosed reagentless electroanalytical method is based on these interactions.

An evaluation was performed regarding the electrochemical signal (potential difference) provided by the inventive potentiometric biodegradable BC-based biosensor through dose-response curves with low concentrations of virus. FIG. 4A shows potentiometric responses of the BC-based biosensor for concentrations ranging from 10.0 zg mL⁻¹ to 1.0 μg mL⁻¹ SARS-CoV-2 SP in 0.1 mol L⁻¹ PBS (pH=7.4). FIG. 4B depicts dose-response curve obtained from ΔE (V) values [subtracted from the blank values (ΔE (V)=Eₛₐₘₚₗₑ−E_{blank})] in the function of the logarithm of the SP concentration. FIG. 4C shows potentiometric responses for SARS-CoV-2 detection in a concentration range from 1×10⁻¹ copies μL⁻¹ to 1×10⁵ copies μL⁻¹. FIG. 4D provides a dose-response curve obtained from ΔE (V) values [subtracted from the blank values (ΔE (V)=Eₛₐₘₚₗₑ−E_{blank})] as a function of the logarithm of the SARS-CoV-2 concentration. All potentiometric measurements were carried out after the incubation (7 min) of 10 μL of SP or SARS-CoV-2 samples on the surface of the BC-based biosensor. The measurements were recorded in triplicate in 0.1 mol L⁻¹ PBS medium (pH=7.4) for 300 seconds.

The measurements were recorded by dropping 10 μL of SARS-CoV-2 SP or clinical samples onto the surface of the biosensor and incubating it for 7 minutes before each measure. The biosensor required at least 30 s to provide a stable potential difference response in the presence of SARS-CoV-2 SP to stabilize the accumulated charge (FIG. 4A, providing potentiometric responses of the BC-based biosensor for concentrations ranging from 10.0 zg mL⁻¹ to 1.0 μg mL⁻¹ SARS-CoV-2 SP in 0.1 mol L⁻¹ PBS (pH=7.4)). All analytical curves were plotted as a potential difference, ΔE:

ΔE=Eₛₐₘₚₗₑ−E_{blank} (Eq.1)

where Eₛₐₘₚₗₑ is the potential measured in the presence of SARS-CoV-2 and E_{blank} is the potential obtained for the blank, i.e., 0.1 mol L⁻¹ PBS at pH=7.4. The electrical potential was sampled at 3 minutes for quantitative purposes to ensure a stable response. The signal for ΔE increased with the increase in the concentration of SARS-CoV-2 SP over the concentration range studied of 10.0 zg mL⁻¹ to 1.0 μg mL⁻¹ in 0.1 mol L⁻¹ PBS at pH=7.4 (FIGS. 4A and 4B).

Next, titered samples with B.1 SARS-CoV-2 concentrations ranging from 1×10⁻¹ copies μL⁻¹ to 1×10⁵ copies μL⁻¹ were analyzed (FIG. 4C), and a dose-response curve was obtained by measuring ΔE as a function of the logarithm of the B.1 SARS-CoV-2 concentration (FIG. 4D). The ΔE response increased from 10⁻¹ to 10³ copies μL⁻¹ and after that reached a plateau, probably due to the limitation of recognizing sites leading to response saturation.

The limits of detection (LOD) and quantification (LOQ) of the electrochemical device were calculated based on the four-parameter logistic (4PL) method, which is commonly employed for bioassays that use binding interactions. Applying equations 2 and 3, we obtained an LOD of 4.26×10⁻¹⁸ g mL⁻¹ and an LOQ of 1.42×10⁻¹⁷ g mL⁻¹ for SARS-CoV-2 SP (FIG. 4B), and an LOD of 0.05 copies μL⁻¹ and an LOQ of 0.17 viral RNA copies μL⁻¹ (FIG. 4D). These analytical parameters indicate that the sensitivity of the present biosensing approach is similar to that of the RT-qPCR technique.

L_{C}=μ_{blank}+t(1−α,n−1)σ_{blank} (Eq. 2))

where L_{C} is a value of blank limit, μ_{blank} is the mean of signal intensities for n blank (negative control) replicates, σ_{blank} is the standard deviation of blank replicates, and t(1−α, n−1) is the 1—α percentile of the t-distribution given n−1 degrees of freedom, α=β=0.05 significance levels.

LOD=L_{C}+t[1−β,m(n−1)]σₜₑₛₜ (Eq. 3)

where L_{d} is the LOD in the signal domain, σₜₑₛₜ is the pooled standard deviation of n test replicates and t[1−β, m(n−1)] is the 1−β percentile of the t-distribution given m(n−1) degrees of freedom. Again, the evaluation was set to σ=β=0.05, but these significance levels can be chosen properly for each study.

A comparison of sensing methods for SARS-CoV-2 was performed, and the inventive device provided the lowest LOD for SARS-CoV-2 SP solution (LOD=4.26×10⁻¹⁸ g mL⁻¹) and gave results in a short time. The testing time was set as 10 minutes, which included 7 minutes for incubation of the sample and 3 minutes for potentiometric analysis (ΔE sampled at 3 min).

Cross-reactivity, reproducibility, and potential stability assays. To investigate the specificity of the instant biosensing electrochemical device for SARS-CoV-2, it was applied to other viruses and viral antigens under the same optimized experimental conditions. In addition to SARS-CoV-2, we tested four other viruses (H₁N₁, Influenza—A/California/2009; Influenza B—B/Colorado; MHV—murine hepatitis virus; and HSV2—herpes simplex virus-2) and three antigenic preparations (corresponding to heat-inactivated Zika virus and yellow fever, and gamma-irradiated Ebola virus). All experiments were carried out in triplicate in 0.1 mol L⁻¹ PBS at pH=7.4 for 300 seconds of analysis. 10 μL of each virus or viral antigen was incubated on the biosensor surface for 7 minutes before the potentiometric measurements were taken (FIG. 6). No cross-reactivity was detected in any of the seven samples, which highlights the utility of our sensor for SARS-CoV-2 detection and COVID-19 diagnosis.

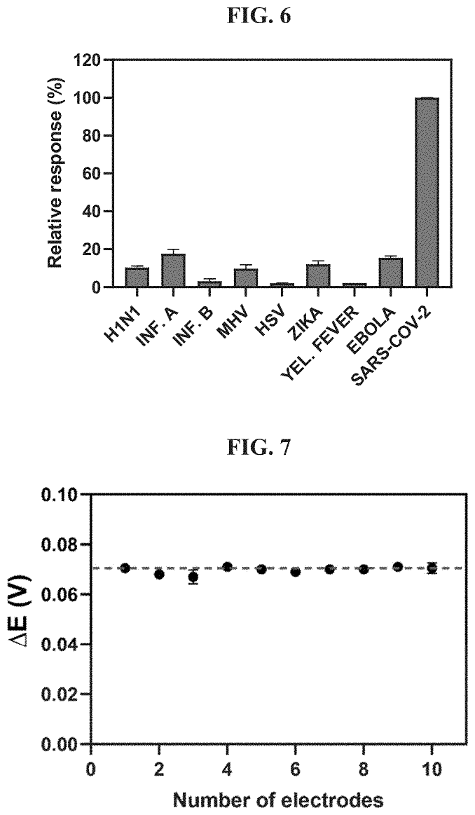

Reproducibility assays were carried out to ensure that different test batches of SARS-CoV-2 performed similarly. For this study, potentiometric measurements were recorded of 1×10¹ copies μL⁻¹ of SARS-CoV-2 prepared in a virus transportation medium (VTM) over 7 minutes of incubation time. The relative standard deviation (RSD) obtained with 10 biosensors representing different fabrication batches was 3.78%, indicating that the present fabrication method and functionalization protocol were highly reproducible (FIG. 7). The observed reproducibility indicates that the fabrication of the inventive device is highly scalable and can be developed to provide on-demand testing at the point of care.

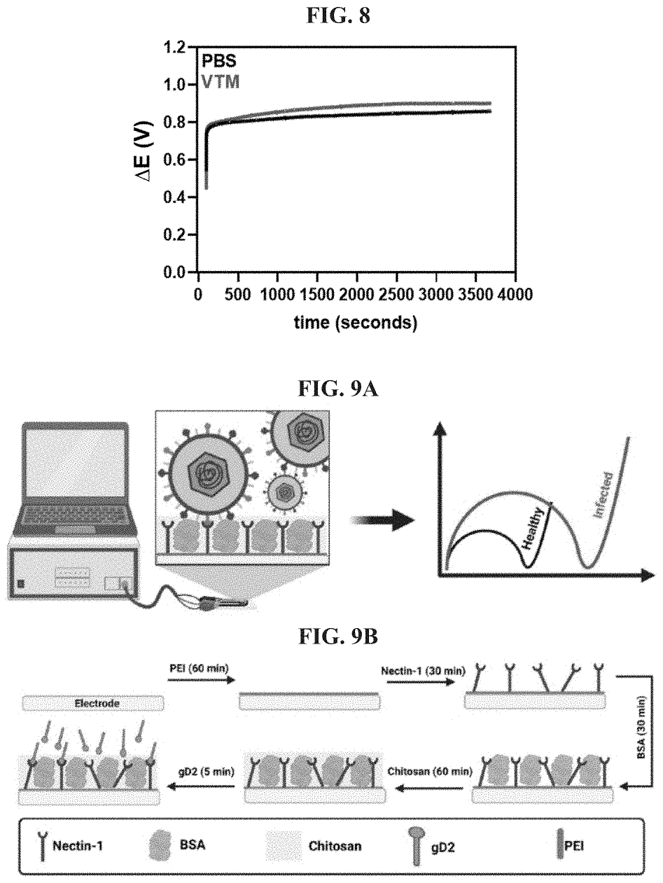

The stability of the biosensor was evaluated potentiometrically using 0.1 mol L⁻¹ PBS (pH=7.4) and VTM for 60 minutes (FIG. 8). The results indicate that the biosensor achieve high stability after 2 minutes, with a low drift response (<4%) over the evaluated period for PBS medium. When VTM was used, a drift response on the electrical potential was noted for a long analysis period (>500s), which may be related to the fouling of the electrode surface by proteins, antibiotics, or other biomolecules present in VTM composition. Therefore, PBS was selected as the optimal medium to carry out all potentiometric tests and sample analyses.

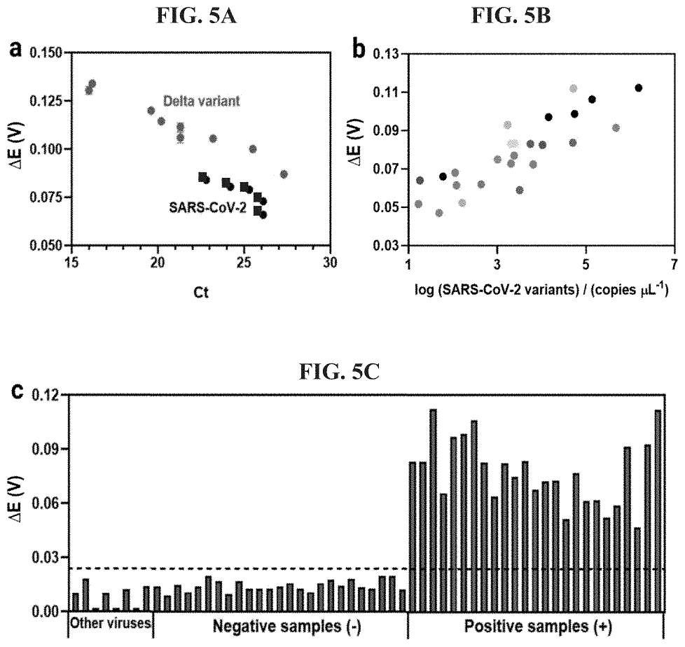

Detection of SARS-CoV-2 in clinical samples. Using the optimized experimental conditions, the present biodegradable electrochemical biosensor was applied to the analysis of 15 OP/NP clinical samples, 5 of which had the original SARS-CoV-2 strain and 10 of which had the SARS-CoV-2 delta variant. FIG. 5A provides the electrochemical response obtained for 5 clinical samples containing original SARS-CoV-2 strain (black circles) and 10 clinical samples containing SARS-CoV-2 delta variant (B.1.617.2, red circles) as a function of Ct values. FIG. 5B shows the potential difference, ΔE, obtained using the modified electrode for another 12 lineages of SARS-CoV-2 as a function of the RNA concentration (copies μL⁻¹) provided by the RT-PCR method, (black ●) B.1, (red ●) B.1.291, (dark blue ●) B.1.369, (pink ●) B.1.340, (green ●) B.1.243, (brown ●) B.1.311, (gray ●) B.1.1.304, (purple ●) B.1.1.317, (orange ●) B.1.2, (light blue ●) B.1.1.7, (light purple ●) B.1.240, (yellow ●) B.1.350. FIG. 5C provides a comparison of the electrochemical response obtained by the cross-reactivity studies (grey bars), 25 SARS-CoV-2 negative clinical samples (red bars), and 25 positive SARS-CoV-2 clinical samples containing different lineages (blue bars). The dotted line indicates the cut-off value of ΔE (V) (ΔE (V)=Eₛₐₘₚₗₑ−E_{blank}) response established to indicate whether the sample was positive for SARS-CoV-2 variants as determined by the inventive biosensor.

Viral loads in the samples ranged widely, with cycle threshold (Ct) values varying from 14.0 to 27.3 (FIG. 5A and Table 1).

TABLE 1

| ID Sample | ΔE (V) | Ct |
|---|---|---|
| SARS-COV-2 (8) | 0.084 | 22.8 |
| SARS-COV-2 (27) | 0.081 | 24.2 |
| SARS-COV-2 (20) | 0.079 | 25.3 |
| SARS-COV-2 (30) | 0.073 | 26.1 |
| SARS-COV-2 (2) | 0.066 | 26.1 |
| Delta 1 | 0.136 | 14.0 |
| Delta 2 | 0.131 | 16.0 |
| Delta 3 | 0.134 | 16.2 |
| Delta 4 | 0.120 | 19.6 |
| Delta 5 | 0.115 | 20.2 |
| Delta 6 | 0.106 | 21.3 |
| Delta 7 | 0.112 | 21.3 |
| Delta 8 | 0.106 | 23.2 |
| Delta 9 | 0.100 | 25.5 |
| Delta 10 | 0.081 | 27.3 |

The biosensor detected SARS-CoV-2 samples and delta variants in all 15 clinical samples analyzed. The ACE2-based biosensor provided a higher analytical response, i.e., increased potential difference, for the SARS-CoV-2 delta variant samples compared to original strain samples with similar Ct values, which may be associated with the higher affinity of their mutated SP with the ACE2 receptor.

In order to evaluate the efficacy and robustness of the biosensor for COVID-19 diagnosis, another set of 50 NP/OP clinical samples was tested, 25 of which were positive NP/OP samples containing 12 SARS-CoV-2 variants of different lineages and 25 of which were negative NP/OP clinical samples (Table 2) obtained, after heat-inactivation, from patients from the Hospital of the University of Pennsylvania (HUP).

TABLE 2

| | ID sample | Lineage | ΔE (V) | Copies μL⁻¹ |
|---|---|---|---|---|
| Positive | 228 | B.1.350 | 0.083 | 2.04 × 10³ |
| Samples | 263 | B.1.350 | 0.083 | 2.47 × 10³ |
| | 266 | B.1 | 0.112 | 1.53 × 10⁶ |
| | 269 | B.1 | 0.066 | 5.97 × 10¹ |
| | 272 | B.1 | 0.097 | 1.43 × 10⁴ |
| | 346 | B.1 | 0.098 | 5.52 × 10⁴ |
| | 373 | B.1 | 0.106 | 1.36 × 10⁵ |
| | 290 | B.1.291 | 0.083 | 5.58 × 10³ |
| | 328 | B.1.369 | 0.064 | 1.80 × 10¹ |
| | 369 | B.1.369 | 0.082 | 1.04 × 10⁴ |
| | 334 | B.1.340 | 0.075 | 1.01 × 10³ |
| | 348 | B.1.240 | 0.083 | 5.04 × 10⁴ |
| | 380 | B.1.243 | 0.068 | 1.12 × 10² |
| | 408 | B.1.243 | 0.072 | 6.43 × 10³ |
| | 423 | B.1.243 | 0.072 | 2.03 × 10³ |
| | 428 | B.1.243 | 0.051 | 1.67 × 10¹ |
| | 444 | B.1.243 | 0.077 | 2.39 × 10³ |
| | 452 | B.1.243 | 0.061 | 1.20 × 10² |
| | 381 | B.1.311 | 0.062 | 4.33 × 10² |
| | 385 | B.1.1.304 | 0.052 | 1.61 × 10² |
| | 391 | B.1.1.317 | 0.059 | 3.18 × 10³ |
| | 406 | B.1.2 | 0.091 | 4.74 × 10⁵ |
| | 455 | B.1.2 | 0.047 | 4.89 × 10¹ |
| | 459 | B.1.1.7 | 0.093 | 1.70 × 10³ |
| | 460 | B.1.1.7 | 0.112 | 5.20 × 10⁴ |
| Negative | 57 | — | 0.014 | 0 |
| Samples | 58 | — | 0.009 | 0 |
| Negative | 59 | — | 0.015 | 0 |
| | 60 | — | 0.011 | 0 |
| | 61 | — | 0.014 | 0 |
| | 62 | — | 0.021 | 0 |
| | 63 | — | 0.017 | 0 |
| | 64 | — | 0.011 | 0 |
| | 65 | — | 0.017 | 0 |
| | 66 | — | 0.013 | 0 |
| | 67 | — | 0.013 | 0 |
| | 68 | — | 0.013 | 0 |
| | 69 | — | 0.014 | 0 |
| | 70 | — | 0.016 | 0 |
| | 71 | — | 0.013 | 0 |
| | 72 | — | 0.011 | 0 |
| | 73 | — | 0.016 | 0 |
| | 74 | — | 0.018 | 0 |
| | 75 | — | 0.014 | 0 |
| | 76 | — | 0.018 | 0 |
| | 77 | — | 0.014 | 0 |
| | 78 | — | 0.012 | 0 |
| | 79 | — | 0.019 | 0 |
| | 80 | — | 0.019 | 0 |
| | 81 | — | 0.012 | 0 |

All the lineages were confirmed by RT-PCR. The cut-off value of the inventive biosensor was set as ΔE>0.025 V as positive for SARS-CoV-2, and ΔE<0.025 V as negative (FIG. 5C). The cut-off value was based on the analytical signal obtained for the lowest quantity of virus analyzed (FIG. 4D). In addition to the 10 NP/OP clinical samples of the delta variant noted above (FIG. 5A), the present BC-based potentiometric biosensor accurately detected the virus in the 25 positive clinical samples containing 12 different SARS-CoV-2 lineages, which suggests that the method would not require additional adaptation for the detection of new SARS-CoV-2 variants, as long as ACE2 remains the entry point into human cells for the mutated virus. There was also a close correlation between the analytical response (ΔE) and the concentration of virus present in the clinical samples (FIG. 5B), highlighting the potential of the present biosensor for both rapid detection of COVID-19 and monitoring the infection status (viral loads) of patients.

New variants of SARS-CoV-2 are likely to continue to emerge in the months and years ahead, and that inexpensive sensors for the detection of this virus will be needed to gather data on outbreaks and to diagnose cases. Here, the robustness and accuracy of a BC-based biosensor were evaluated by analyzing 65 NP/OP clinical samples (40 positive NP/OP samples from 13 SARS-CoV-2 lineages and 25 negative NP/OP samples; Tables 1 and 2). The accuracy of detection of this range of samples suggests that the inventive device would not require additional adaptation to detect emerging SARS-CoV-2 variants, as long as the newly mutated virus interacted with ACE2 to enable its entry into human cells. Based on its outstanding analytical parameters (high selectivity, reproducibility, specificity, and accuracy), low cost, simplicity, and biodegradability, the present device is well suited for frequent testing at the point of need. Thus, the inventive devices may help to prevent outbreaks in countries where the SARS-CoV-2 vaccination rates are low but frequent testing is feasible and sanitary practices are adequate.

### Methods

i. Electrochemical measurements. For electrochemical characterization of the electrodes in each step of modification, the CV technique was used in a potential window ranging from 0.7 to −0.3 V and with a scan rate of 50 mV s⁻¹. EIS experiments were carried out at frequencies ranging from 1×10⁵ Hz to 0.1 Hz using an amplitude of 10 mV, and under open circuit potential (OCP). The electrochemical studies were recorded using 0.1 mol L⁻¹ KCl solution containing 5.0 mmol L⁻¹ of the redox probe [Fe(CN)₆]^{3-/4-} solution. Potentiometric measurements were carried out in a time interval of 300 seconds using 0.1 mol L⁻¹ PBS (pH=7.4). A MULTI AUTOLAB M101 potentiostat with six channels, controlled by the NOVA 2.1 software, was used for all the electrochemical measurements. Experiments were carried out at room temperature, 25±3° C.

ii. SARS-CoV-2 biosensing. For SARS-CoV-2 biosensing, 10.0 μL of 0.1 mol L⁻¹ PBS (pH=7.4) or VTM containing either SP or SARS-CoV-2 samples was applied to the biosensor surface and the device was incubated at room temperature for 7 minutes. Following incubation, the electrochemical cell was gently washed with 0.1 mol L⁻¹ PBS (pH=7.4) to remove the unbound virus and sample. Then, 200 μL, of the 0.1 mol L⁻¹ PBS (pH=7.4) was used for potentiometric measurements and the potential value (E) was obtained. The calibration curves were obtained in the concentration range from 10.0 zg mL⁻¹ to 1.0 μg mL⁻¹ SARS-CoV-2 SP in 0.1 mol L⁻¹ PBS (pH=7.4).

iii. Reproducibility, stability, and cross-reactivity studies. To carry out the reproducibility study, the potential response was obtained by exposing 6 electrodes (from different batches) to 1×10¹ copies μL⁻¹ of SARS-CoV-2 prepared in VTM for 7 minutes. The stability of the electrode response was potentiometrically evaluated in both 0.1 mol L⁻¹ PBS and VTM for 1 hour. Cross-reactivity studies were performed with the following viral strains, all at 10⁵ PFU mL⁻¹: H₁N₁, Influenza—A/California/2009; Influenza B—B/Colorado; MHV—murine hepatitis virus; HSV2—herpes simplex virus-2. Cross-reactivity studies were also performed with heat-inactivated antigenic preparations of Zika virus (viral genome copy number: 1.1×10⁷ copies μL⁻¹), yellow fever virus (viral genome copy number: 1.8×10⁴ copies μL⁻¹), and Ebola virus (viral genome copy number: 1.1×10⁷ copies μL⁻¹), obtained from BEI Resources®. All the experiments were carried out by combining the viral sample with 0.1 mol L⁻¹ PBS for 300 seconds of analysis, and 10 μL, of each virus (or antigenic preparation) was incubated on the biosensor surface for 7 minutes before the potentiometric measurements were made.

iv. Clinical sample analysis. NP/OP swab patient samples were heat-inactivated prior to analysis. Of the 65 NP/OP samples analyzed in this study, 40 were positive and 25 were negative for SARS-CoV-2 when tested by the RT-PCR method. The 25 negative clinical samples were acquired from the Hospital of the University of Pennsylvania (IRB protocol 844145). The 40 positive SARS-CoV-2 samples containing 13 variants: B.1.350, B.1.340, B.1, B.1.291, B.1.369, B.1.240, B.1.243, B.1.311, B.1.1.304, B.1.1.317, B.1.2, B.1.1.7 (alpha variant), and B.1.617.2 (delta variant) were obtained from under IRB protocol 823392. WA cut-off value of potential response (ΔE) was set to higher than 25 mV to express a positive diagnostic result, in accordance with the analytical response obtained for the lowest detected concentration of SARS-CoV-2 (10⁴ copies μL⁻¹) in the dose-response curve (FIG. 4D), i.e., samples that exhibited ΔE>25 mV were considered positive for SARS-CoV-2 (Table 2 and FIG. 5C). The concentration range obtained by RT-PCR for the delta variant and the other 12 SARS-CoV-2 variants in the clinical samples ranged from 14 to 27.3 cycle threshold (Ct) and from 1.67×10¹ to 1.53×10⁶ RNA copies μL⁻¹, respectively.

### Example 3—Fabrication of HSV Biosensor

An HSV biosensor that was functionalized with nectin-1 was prepared. Electrochemical impedance spectroscopy (EIS) was used for the transduction of biosensor response, i.e., the selective binding between the nectin-1 bioreceptor immobilized on the electrode surface and the gD2 glycoproteins from HSV-2. The binding between nectin-1 and gD2 changes the interfacial electron transfer kinetics between ferricyanide/ferrocyanide (i.e., the redox probe used) and the electrode. The altered kinetics, in turn, can be detected by monitoring the increase in resistance to charge transfer (R_{CT}), indicating a positive diagnostic result for HSV-2 infection (FIG. 9A). Each functionalization step was studied in order to generate a reliable, ultrasensitive, and robust biosensor that presents original functional materials for HSV-2 diagnosis (FIG. 9B). The R_{CT} values were extracted by application of the Randles equivalent electrical circuit.

All data from the optimization studies and analytical curves were plotted using the normalized R_{CT} response, as defined by the following equation:

$$Normalized {R}_{CT} = \frac{Z - {Z}_{0}}{{Z}_{0}} \qquad ( Eq \text{.1} )$$

where Z is the R_{CT} value obtained after incubating the electrode surface with gD2 or HSV-2 samples, and Z₀ is the R_{CT} value of the analytical blank solution [i.e., PBS or Dulbecco's Modified Eagle Medium (DMEM) with 5% fetal bovine serum (FBS)]. The normalization process of R_{CT} corrects variation in the sensor response, which may be caused by analyst operation and temperature fluctuations when testing. Thus, normalization facilitates the eventual use of the sensor at decentralized testing sites.

The electrochemical sensors (3-electrode configuration) were manufactured by a screen-printing technique on phenolic paper circuit board material, as a low-cost and convenient platform. Electrically conductive carbon and Ag/AgCl inks (Creative Materials, USA) were employed to construct the working (WE)/auxiliary (ΔE) and reference (RE) electrodes, respectively. After a curing step of 30 min at 100° C., the material was cut into 2.5×2.0 cm pieces, and their geometrical area was delimited using dielectric tape.

Initially, to generate a robust and sensitive biosensor, two strategies were evaluated to modify the working electrode (WE) and enable the anchoring of the nectin-1 bioreceptor. In the first approach, the WE surface was coated with glutaraldehyde (GA), a dialdehyde used to anchor biomolecules through their N-terminal groups; for the second approach, the WE was modified with PEI, a conductive polymer containing amino functional groups enabling the attachment of biomolecules through their carboxylic acid and ester groups.

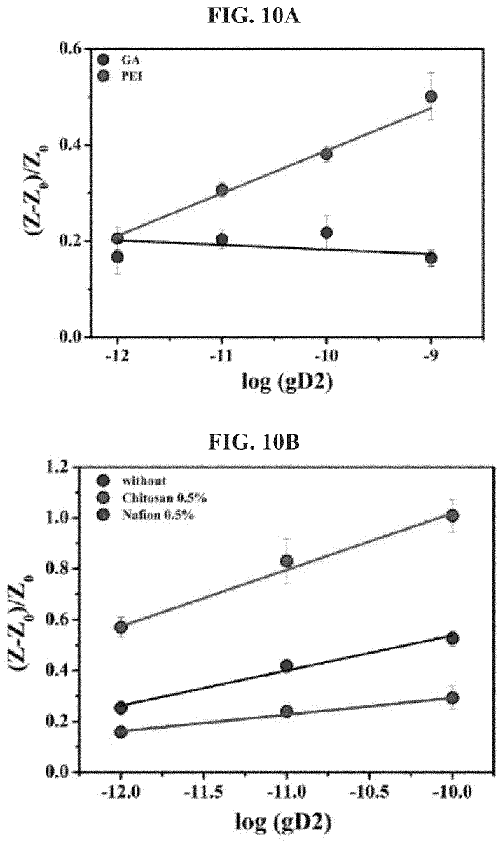

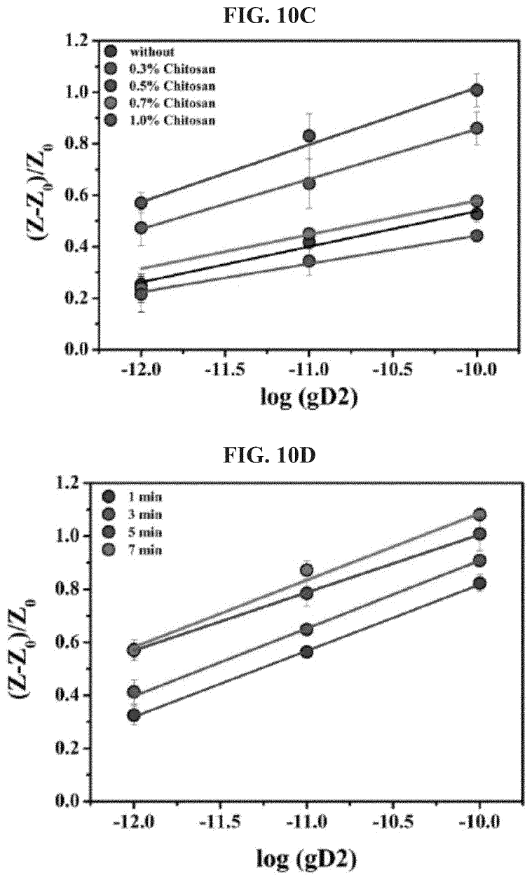

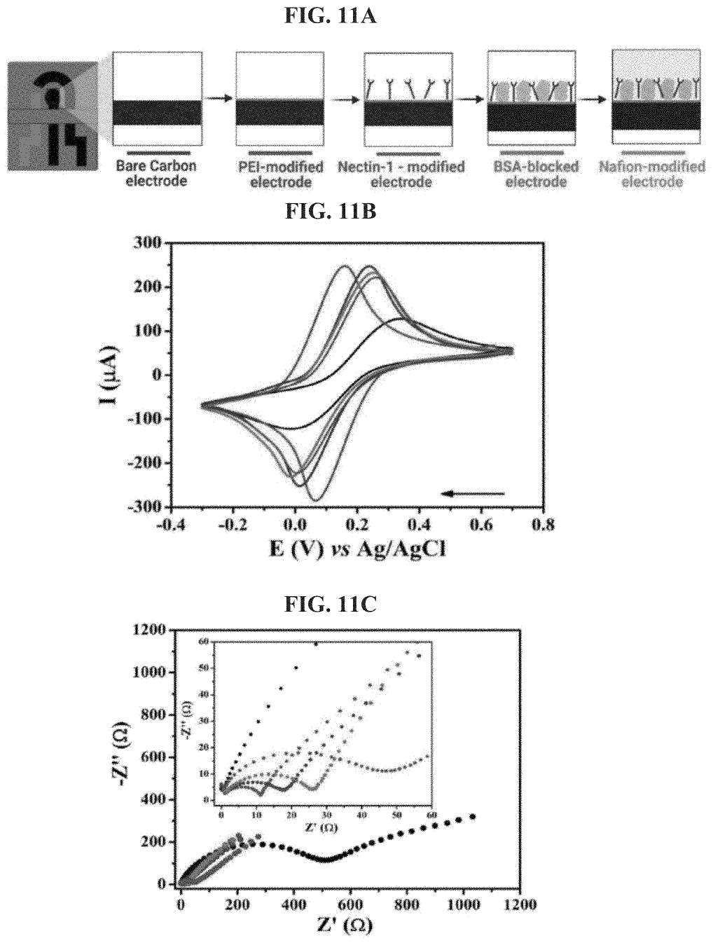

FIGS. 10A-10D depict the results of the characterization of the biosensor. FIG. 10A pertains to anchoring of nectin-1 using 25% (m/v) GA (black circles) and 1 mg mL PEI (red circles). Optimal results were obtained when the substrate was modified with PEI to enable the anchoring of the nectin-1 receptor through the —COOH terminal group. FIG. 10B depicts the analytical response of the biosensor when fabricated without an additional membrane layer (black circles), modified with 0.5% (m/v) chitosan (red circles), and modified with 0.5% (m/v) Nafion (blue circles). The highest sensitivity was obtained when the biosensor was modified with 0.5% (m/v) chitosan. FIG. 10C shows the effect of chitosan concentration on biosensor sensitivity: 0.0% (black circles), 0.3% (m/v; red circles), 0.5% (m/v; blue circles), 0.7% (m/v; pink circles), and 1.0% (m/v; green circles). Chitosan at 0.5% (m/v) provided the highest detectability maintaining the lowest reagent-to-usage ratio; thus, this condition was selected for subsequent measurements. FIG. 10D shows the results of incubation time experiments between gD2 and the modified electrochemical biosensor. Calibration curves were generated using gD2 at concentrations ranging from 1 pg mL⁻¹ to 0.1 ng mL⁻¹ and incubation times ranging from 1 to 7 minutes. No significantly increased differences in the detectability of gD2 were observed for incubation periods longer than 5 minutes; thus, this incubation time was selected for subsequent work. All experiments were carried out at room temperature and obtained through calibration curves for gD2 at a concentration range between 1.0 pg mL⁻¹ and 0.1 ng mL⁻¹. All EIS measurements were recorded at open circuit potential at the frequency range of 1×10⁵ Hz to 0.1 Hz and using an amplitude of 10 mV in the following medium: 5 mmol L⁻¹ [Fe(CN)₆]^{-3/-4} in 0.1 mol L⁻¹ KCl solution.

Using GA as a modifier did not provide significant discrimination of the analytical signal (R_{CT}) at the concentrations of gD2 tested (10⁻¹²-10⁻⁹ g mL⁻¹, FIG. 10A). This result can be explained by the partial obstruction, or steric effect of the active sites, present in the domain of the receptor when this immobilization strategy was used, which may have hindered the effective interaction with the viral particle. This hypothesis was confirmed by the observation that the PEI modification allowed detection of the binding interactions between nectin-1 and gD2, yielding the high sensitivity seen in the analytical curve (FIG. 10A). The binding of the C-terminal of nectin-1 to the PEI-modified surface left the —NH groups of the former free for gD2 with which to interact.

The main fabrication, modification, and functionalization steps of the biosensor using PEI was investigated. First, the working electrode was modified with 4.0 μL of 1.0 mg mL⁻¹ PEI solution, by drop-casting, and incubated for 60 min at 37° C. This procedure generates —NH functional groups on the carbon electrode surface. Then, 4.6 μL of 0.13 mg mL⁻¹ of the nectin-1 receptor, containing a mixture of 25.0 mmol L⁻¹ EDC+50.0 mmol L⁻¹ NHS, was deposited on the surface of the PEI-modified WE, and the biosensor was incubated for 30 min at 37° C. The carboxyl groups on nectin-1, when exposed to EDC-NHS, are activated to form a stable ester, which undergoes a nucleophilic addition with the amino groups on the PEI-modified WE, such that a stable amide bond is formed between the PEI-modified carbon electrode and nectin-1. Subsequently, the remaining unmodified sites of the electrode surface were blocked with 4.0 μL of a 1.0% (m/v) BSA solution. In the last step, 4.0 μL of 0.5% (m/v) chitosan was dropped on the surface of the nectin-1-modified WE.

After selecting PEI as the immobilization strategy for nectin-1, the use of two types of permeable membranes, namely Nafion® and chitosan, was investigated. Analytical curves ranging from 1×10⁻¹² to 1×10-10⁻¹⁰ g mL⁻¹ of gD2 in 0.1 mol L⁻¹ of PBS (pH=7.4) were constructed. Experiments were performed in triplicate to compare 3 strategies: i) without a permeable membrane, ii) with 0.5% Nafion, and iii) with 0.5% chitosan (FIG. 10B). According to these results, the electrochemical biosensor modified with chitosan 0.5% (m/v) presented a sensitivity of 0.222, which is 1.6-fold higher than the biosensor without any semipermeable membrane (sensitivity of 0.138) and 2.74-fold higher than the biosensor with Nafion (sensitivity of 0.081). The increase in sensitivity is associated with the preconcentration features of the glycoprotein gD2 during the incubation period, which is trapped close to the bioreceptor, enabling a larger number of binding events, and enhancing the detectability of our method (FIG. 10B). In addition, the positive charges displayed by chitosan in the acidic medium can preconcentrate [Fe(CN)₆]^{3-/4-}, i.e., the anionic redox probe, into the polymeric layer, enhancing the electrochemical response. Given these results, the proportion of chitosan on the modified biosensor was investigated, since it directly impacts membrane thickness. The experiments revealed that 0.5% (m/v) of chitosan provided the highest impedimetric responses and analytical sensitivity since higher concentrations provided lower detectability (FIG. 10C). Thus, 0.5% (m/v) of chitosan was selected for further studies.

Subsequently, the optimal incubation time of either gD2 or viral samples with the surface of the biosensor was investigated to obtain a compromise between analytical frequency and sensitivity for HSV-2 detection. The evaluation was based on the analytical sensitivity (slope) parameter obtained by analytical curves, determined in triplicate, at concentrations of gD2 ranging from 10⁻¹² to 10⁻¹⁰ g mL⁻¹ (FIG. 10D). By balancing detection ability with the sensitivity values of the dose-response curves while maintaining a short testing time, 5 minutes was selected for the incubation time. These results demonstrate the rapid binding kinetics between gD2 and the immobilized nectin-1 on the electrode surface, underscoring the efficiency of the functionalized biosensor architecture.

### Example 4—Electrochemical Characterization and Analytical Performance of HSV Biosensor

Electrochemical Characterization. For each functionalization step (FIG. 11A), the electrochemical behavior was characterized by CV and EIS (FIGS. 11B and 11C, respectively). CV (FIG. 11B) and Nyquist (FIG. 11C) plots showed that the bare carbon electrode (black line) presented poorly defined redox processes with peak currents (ip) of 133.1±2.5 μA and R_{CT} of 549.4±24.6Ω. The electrochemical performance of the sensor was enhanced by modifying the carbon electrode surface with PEI (red line), as well-defined and intense (251.71±3.17 μA) current peaks were observed for the redox probe with an R_{CT} value of 11.1±1.2Ω. These results were expected, given the high charge transfer generated by the π-electrons of the conductive PEI membrane. Next, nectin-1 was anchored to the electrode surface using the EDC-NHS approach (blue line). The receptor was first immobilized through an amide bond between the amine group from the PEI and the carboxyl groups from nectin-1. This step led to a small increase in the R_{CT} value, to 17.6±2.1 S2, and a slight decrease of the ip, to 247.15±2.56 μA (blue line). Any nonspecific sites of the electrode were blocked by using 0.1% (m/v) BSA solution, resulting in an R_{CT} of 26.3±1.2 S2 and ip of 231.2±3.9 μA (magenta line) due to the introduction of a nonconductive layer on the surface of the electrode. Finally, the electrode surface was modified with a 0.5% (m/v) chitosan permeable membrane to enhance the robustness and sensitivity of the biosensor. This step increased the R_{CT} to 47.5±4.0 S2 and decreased the ip to 222.0±3.3 μA (green line).

All electrochemical measurements were carried out using a mixture of 5.0 mmol L⁻¹ [Fe(CN)₆]³⁻ and [Fe(CN)₆]⁴⁻, as a redox probe, in 0.1 mol L⁻¹ KCl solution. All functionalization steps of the biosensor were characterized by electrochemical Impedance Spectroscopy (EIS), which was also used to quantify the HSV-2 and gD2 concentrations. The frequencies used ranged from 1×10⁵ Hz to 0.1 Hz, and the open circuit potential was applied with an amplitude of 10 mV (vs. Ag/AgCl). For cyclic voltammetry (CV) experiments, the potential ranged from −0.3 to 0.7 V (vs. Ag/AgCl) using a scan rate of 50 mV s⁻¹.

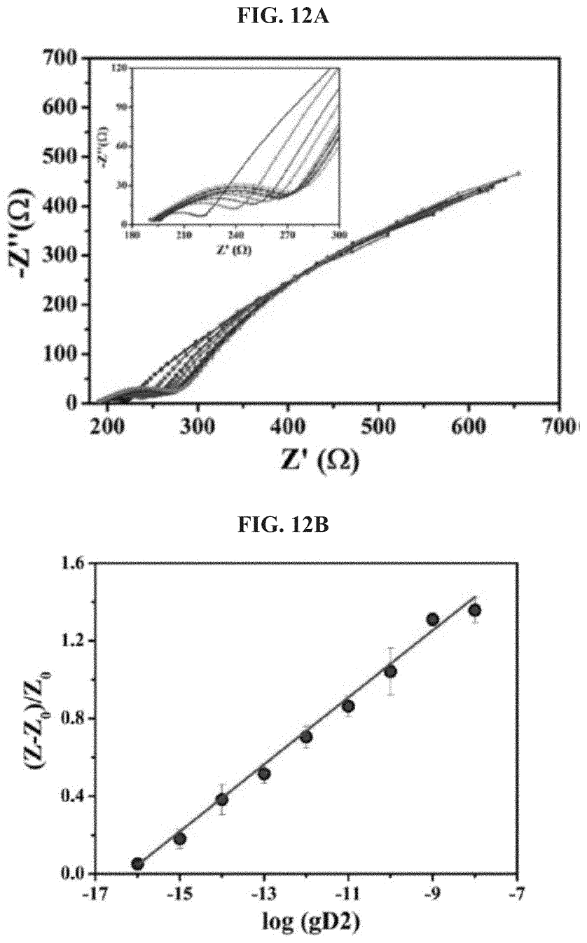

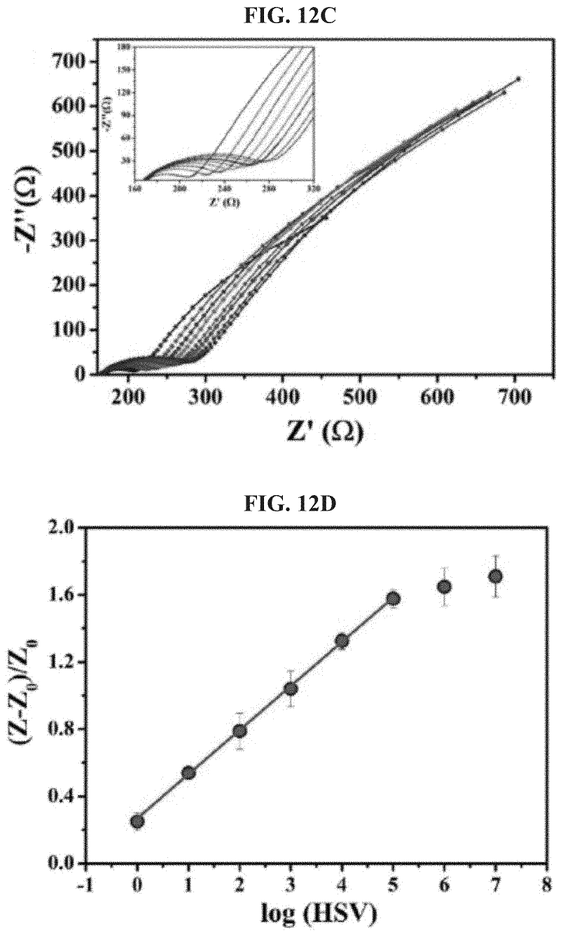

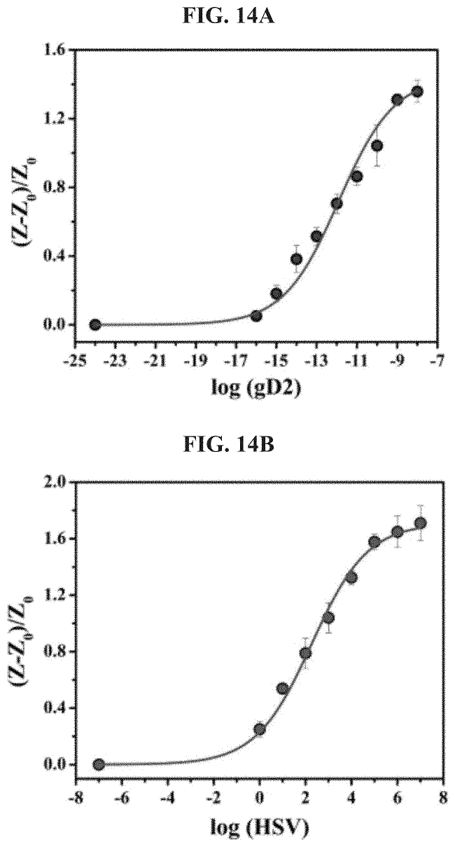

Analytical Performance. EIS was used to quantify free gD2 and HSV-2 virus in 0.1 mol L⁻¹ PBS (pH=7.4). Dose-response curves were built with the previously described experimental conditions (i.e., 1 mg mL⁻¹ PEI, 0.5% chitosan, and 5 minutes of incubation time), and the analytical results were normalized according to Eq. 1. FIG. 12A illustrates Nyquist plots for increased concentrations of gD2 ranging from 0.1 fg mL⁻¹ to 10.0 ng mL⁻¹ in 0.1 mol L⁻¹ PBS (pH=7.4). A linear correlation was observed over the entire range of concentrations evaluated (0.1 fg mL⁻¹ to 10.0 ng mL⁻¹ gD2), when plotted as a logarithm function (FIG. 12B), with a determination coefficient R² of 0.997. The LOD and limit of quantification (LOQ) were calculated as 0.019 fg mL⁻¹ and 0.089 fg mL⁻¹ gD2, respectively. An analytical curve was built for a titered HSV-2 sample at concentrations, in a DMEM medium, ranging from 1×10° to 1×10⁷ PFU mL⁻¹ (FIG. 12C). A linear correlation was observed in the concentration range from 1×10⁰ PFU mL⁻¹ to 1×10⁵ PFU mL⁻¹ with an R²=0.999 (FIG. 12D). LOD and LOQ were calculated as 0.057 PFU mL⁻¹ and 0.210 PFU mL⁻¹ HSV-2, respectively. Three different biosensors were used per experiment, and concentrations were depicted as the logarithmic function of the dose used for gD2 and HSV-2. The four-parameter logistic (4PL) curve (FIGS. 14A, 14B), a method that assesses binding interactions and kinetics, was used to determine the LOD and LOQ values.

Collectively, these experiments highlight the excellent sensitivity displayed by the inventive biosensor, which should provide an early diagnosis of HSV-2 infection in human clinical samples. Another advantage for diagnostic purposes is the short testing time, i.e., 9 minutes, consisting of a 5-minute incubation of the sample on the electrode surface and an additional 4 minutes for the EIS measurements of both the analytical blank and the sample of interest.

In comparison to other approaches reported in the literature, the present disclosure provides the first approach that uses a moiety for detecting the viral glycoprotein gD2 instead of genosensor technology using genetic material for the recognition of HSV. In addition, the presently disclosed HSV sensors presents the fastest testing time, with a very low LOD and a large interval concentration range to detect HSV-2. Furthermore, the devices can be produced inexpensively. Considering the cost of nectin-1 ($800/mg), for example, the final cost to assemble each HSV biosensor was exactly $1.00: $0.12 for electrode fabrication+$0.40 for all the chemicals used in the functionalization step (PEI+EDC+NHS+BSA+Chitosan)+$0.48 for nectin-1. Because the present biosensors are low-cost, their production is potentially highly scalable.

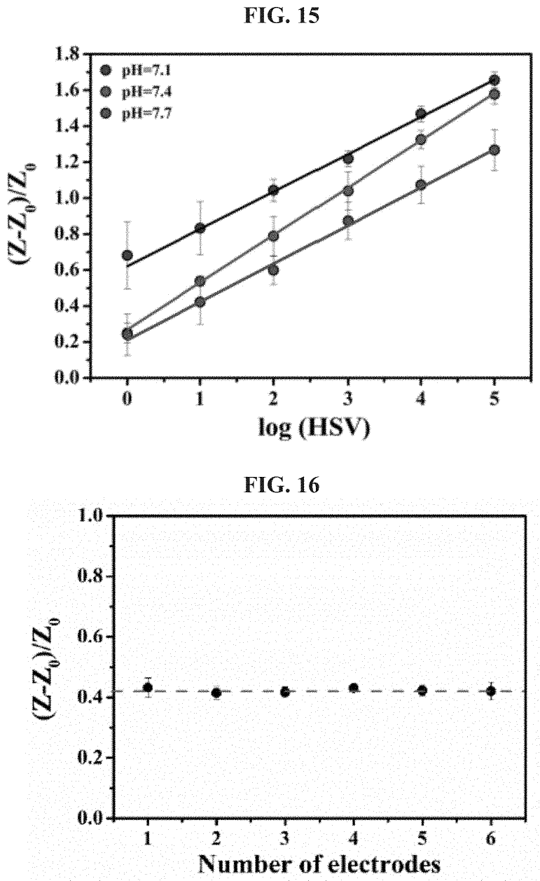

The effect on the biosensor's electrochemical response of adjusting the pH of the medium to a pH that is close to physiological conditions was also studied (FIG. 15). DMEM medium was used to dilute the titered virus samples, and each pH value was adjusted to the range of 7.1-7.7 and tested using the optimized protocol previously described. When the pH of DMEM was 7.1, the biosensor exhibited a high detectability and a sensitivity of 0.212±0.008. At pH 7.4, sensitivity increased to 0.263±0.003. Finally, when the pH of DMEM was 7.7, the analytical sensitivity of the biosensor decreased (0.207±0.008). These data can be explained by conformational changes of the biomolecules induced by differences in pH which, in turn, affect the binding of gD2 to the nectin-1 receptor. These results indicate the importance of adjusting the pH of biofluids for diagnostic purposes, for example, by using a buffered medium, since genital samples are usually acidic. These results are consistent with previous studies evaluating the effect of pH changes on the interaction between gD2 and the nectin-1 receptor, which found that the alkalinity of the medium changed the proximity between the viral bilayer and the host cell membrane, likely affecting the interaction between nectin-1 and gD2. These changes influence the ability of the virus to fuse with and infect the cells.

### Example 5—Reproducibility and Stability Assays

To verify the reproducibility of the proposed method, i.e., to assess whether different batches of biosensors performed similarly, 6 biosensors from different fabrication rounds were evaluated using the same optimized protocol. Briefly, the R_{CT} measures were recorded by EIS using 5 mmol L⁻¹ [Fe(CN)₆]^{-3/-4} after incubating the biosensor with 1×10⁻⁹ g mL⁻¹ of gD2 prepared in 0.1 mol L⁻¹ of PBS (pH=7.4) (FIG. 16). A relative standard deviation (RSD) of 5.12% was obtained, indicating excellent reproducibility of the manufacturing and biofunctionalization protocol. The experiments were carried out by incubating 10 μL of sample diluted in 0.1 mol L⁻¹ PBS (pH=7.4) for 5 minutes before recording each measurement.

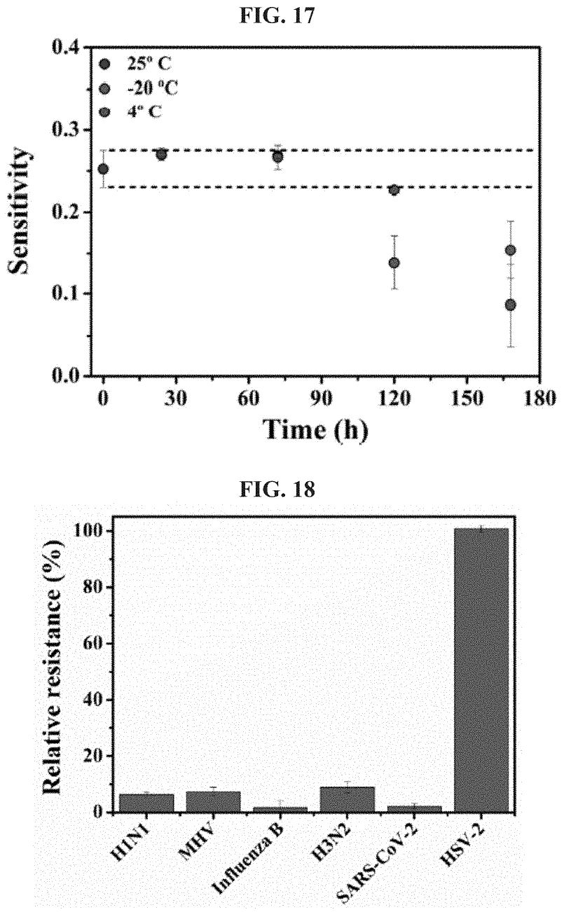

The stability of the electrochemical biosensor, stored in sealed Petri plates at various temperatures (−20° C., 4° C., and 25° C.), was evaluated over 7 days. Analytical curves were built at concentrations ranging from 1×10⁻¹² g mL⁻¹ to 1×10⁻⁹ g mL⁻¹ gD2 in 0.1 mol L⁻¹ PBS, pH 7.4 (FIG. 17). The biosensors did not exhibit stability when stored at room temperature overnight. When stored at −20° C., on the other hand, the biosensors were stable for up to 72 hours, and after 120 hours, the sensitivity decreased to 48% of the initial value. The freezing of the biosensor for prolonged periods may modify the structuring of the functionalized surface, changing its ability to recognize the virus, i.e., the sensitivity. In this regard, electrodes stored at 4° C., the intermediary condition tested, were stable for 120 hours (5 days). The mean sensitivity of the device decreased after 7 days, displaying 40% of the initial performance of the device.

### Example 6—Detection of HSV in Pre-Clinical Animal Model

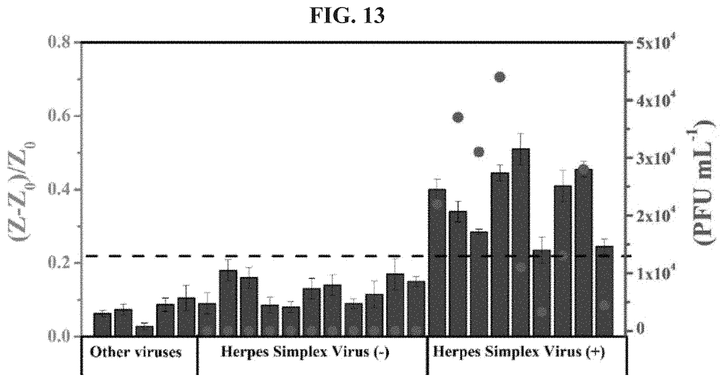

The ability of the biosensor to detect HSV-2 in pre-clinical samples was assessed. Tested blindly, in triplicate (n=3), were 9 HSV-2 positive and 11 negative biofluid samples collected from the vagina of guinea pigs (FIG. 13). All samples were heat-inactivated (56° C. for 1 h) prior to the electrochemical analysis. All samples were obtained from guinea pigs that had been infected two days earlier with HSV-2 or that were uninfected. The biosensor performance is dependent on the cut-off used to discriminate positive and negative samples. A low cut-off can enable the detection of low viral loads but may lead to false positive results. On the other hand, high cut-off values avoid false positive results but limit the detectability of the method, i.e., lead to false negative results. For diagnostic purposes, the cut-off value of our biosensor was set as [(Z−Z₀)/Z₀]>0.22 to identify a positive HSV-2 result, and [(Z−Z₀)/Z₀]<0.22 for negative samples. The cut-off value was based on the analytical signal obtained for the lowest quantity of titered virus analyzed (FIG. 12A).

The biosensors achieved 88.9% sensitivity, 100% specificity, and 95% accuracy for the set of 20 samples evaluated, i.e., the biosensors correctly diagnosed 19/20 samples tested. There was a response variation between the proposed method and the titrated method for sample analyses (FIG. 13). This is likely due to the heat inactivation process prior to performing the electrochemical measurements, since heat inactivation induces viral lysis, generating different amounts of free gD2 or cell fragments containing gD2 that can interact with the nectin-1 present on the surface of the working electrode. In addition, the heating step needs to be carefully performed to avoid denaturation of the glycoproteins, i.e., structural alterations on viral proteins (gD2). Thus, these points prevent an exact correlation between the titrated method and our approach. However, based on the data obtained, the high diagnostic accuracy (95%) observed for the 20 samples tested suggests that the selective biosensor approach is excellent at detecting HSV-2 viral particles in complex samples and thus constitutes a promising alternative to standard methods.

### Example 7—Cross-Reactivity Experiments

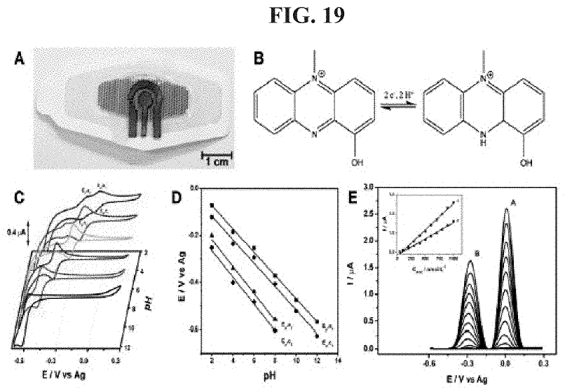

Cross-reactivity experiments were performed to rule out any potential off-target effects of the nectin-1-modified electrode with viruses other than HSV. Selectivity was studied for 5 viruses: H₁N₁ (A/California/2009), Influenza-B/Colorado, H₃N₂, MHV-murine hepatitis virus, and SARS-CoV-2. All experiments were performed using the same optimized conditions as those used for HSV-2 detection. No significant cross-reactivity was detected with any of the viruses tested, as revealed by a relative R_{CT} percentage of up to 12%, which is lower than the cut-off value of 22% established for a positive diagnosis of HSV-2 infection in biofluid samples (FIG. 18). These results, associated with the selectivity observed in the analysis of pre-clinical samples (guinea pig vaginal biofluids) in which no false positives were detected, highlight the robustness and selectivity of the biosensor. However, the glycoprotein D proteins from HSV-1 and HSV-2 have a high degree of identity and both viruses can enter the cell through gD binding to the nectin-1 receptor, which could result in the detection of HSV-1 if that virus were present in genital biofluid, or the clinical sample tested. The present devices can be advantageous to diagnose both HSV-1 and HSV-2 infections. A relevant scenario for the use of such a testing device could be in pregnant women in labor before childbirth if the presence of either HSV-1 or HSV-2 is suspected. Such a diagnosis can help prevent the newborn from acquiring neonatal herpes from an infected mother.

### Example 8: Detection of SARS CoV-2

An electrode is screen-printed onto a paper substrate. The electrode is functionalized with thiol groups. An ACE2 protein that further includes an N-terminus cysteine group is bonded to the thiol-functionalized electrode via disulfide bonds. Bovine serum albumin is used to block the remaining exposed surfaces of the electrode.

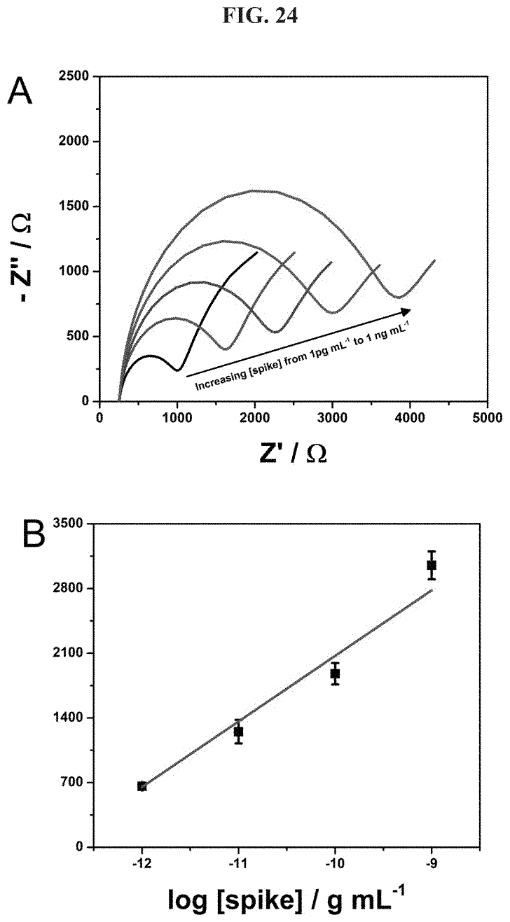

The device comprising the electrode and the substrate is contacted with blood serum from a subject suspected of being infected with SARS CoV-2. A potentiostat is used to deliver a current to the electrode, and the resulting EIS signal is recorded using a Squidstat Plus analyzer at open circuit potential and a frequency range from 10⁵ to 10⁻² Hz using an alternated current signal of 10 mV amplitude. The changes in resistance to charge transfer (R_{CT}), before and after exposure of the electrode to the blood serum is used to provide qualitative and quantitative results that enable COVID-19 diagnosis. FIGS. 24A and 24B provide the results of the assessment.

### Example 9—Device with Portable Potentiostat

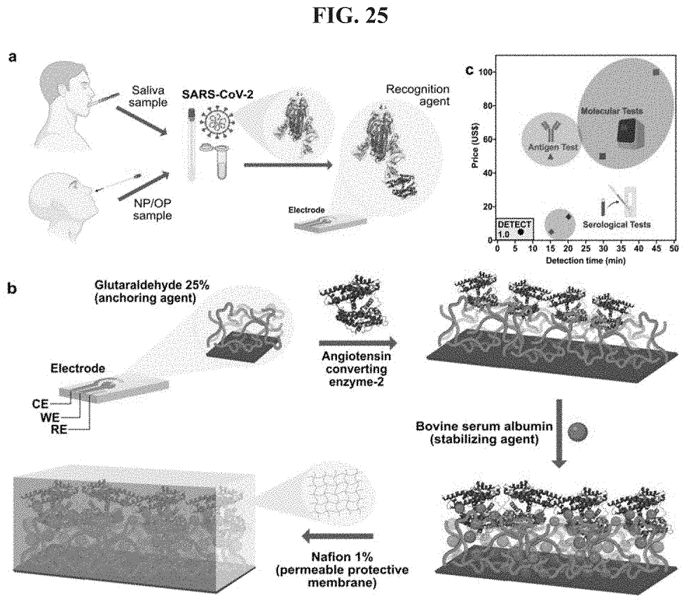

Inventors developed a simple, inexpensive, and rapid test for detection of SARS-CoV-2, dubbed “DETECT 1.0” (DETECT 1.0 (Detection through Electrochemical Technology for Enhanced COVID-19 Testing prototype 1.0) (FIG. 25) The device transformed biochemical information from a specific molecular binding event between the SARS-CoV-2 spike protein (SP) and ACE2 into an electrical signal that can easily be detected.

As illustrated in FIG. 25A-25C, DETECT 1.0 enables diagnosing neat saliva and NP/OP swab samples infected with SARS-CoV-2 (FIG. 25A). FIG. 25B provides a schematic for the preparation of the electrodes. Briefly, the screen-printed electrodes in a three-electrode configuration cell (counter electrode—CE, working electrode—WE, and reference electrode—RE) were printed in phenolic paper circuit board or filter paper with conductive carbon and Ag/AgCl inks. The WE was functionalized with glutaraldehyde to enable anchoring of ACE2, which was stabilized by the addition of bovine serum albumin. Detection was improved by adding a Nafion permeable membrane enabling chemical preconcentration of cation species and protecting the electrode's surface against biofouling with proteins, lipids, and other macromolecules present in biological samples. FIG. 25C provides a cost and detection time comparison matrix between DETECT 1.0 and existing FDA-approved antigen, serological and molecular tests (Government, A. C. (2020). Information of Coronavirus (COVID-19) Testing; Service, R. (2020); Administration, U.S.F.& D. (2020). In Vitro Diagnostics EUAs).

DETECT 1.0 (also referred to herein as DETECT) uses electrochemical impedance spectroscopy (EIS), an electrochemical technique extensively utilized for the characterization of functionalized electrode surfaces and the transduction of biosensors. In our test, the EIS transducer signal reported the selective interaction/binding between the biological receptor immobilized on the electrode surface (i.e., ACE2) and its binding element (i.e., spike protein). The binding between these two molecules causes a change in interfacial electron transfer kinetics between the redox probe, ferricyanide/ferrocyanide in solution and the conducting electrode sites. This electrochemical change is then detectable by monitoring the charge-transfer resistance (R_{CT}), the diameter of the semi-arc on the Nyquist plot, which correlates with the number of targets bound to the receptive surface. The selectivity of an EIS biosensor mostly relies on the specificity between the target and the recognizing bioelement immobilized on the electrode surface and its robustness through the designed architecture surfaces to minimize non-specific binding of the analyte or adsorption of other biomolecules in solution.

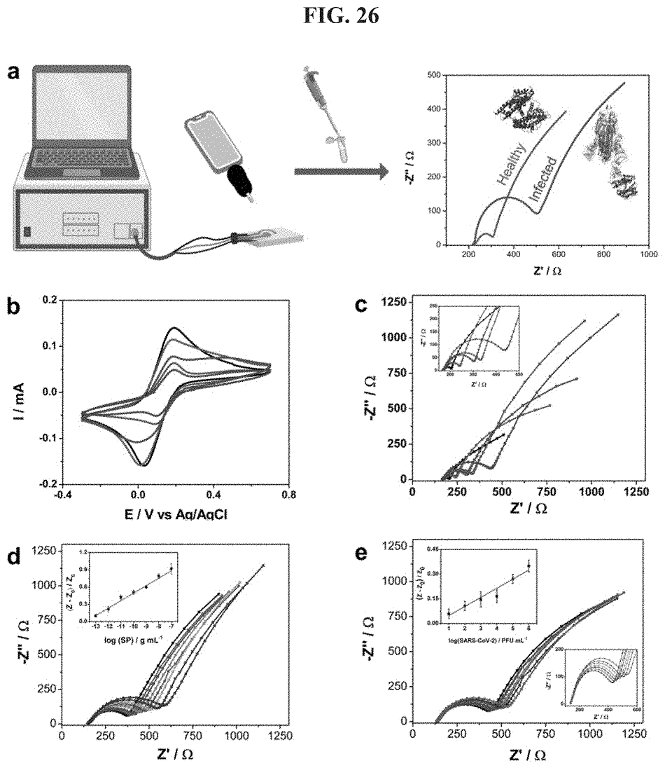

The electrochemical device was designed to explore the remarkable binding affinity of SARS-CoV-2 spike protein (SP) to ACE2, its receptor in the human body. FIGS. 26A-26E provide information concerning the characterization and calibration of the DETECT 1.0 device. FIG. 26A is a schematic representation of the DETECT diagnostic process. FIG. 26B provides a cyclic voltammetry plot, and FIG. 26C provides a Nyquist plot (inset shows the zoomed region of the curve with the semi-arc) of all functionalization steps showing progressive increased resistivity between the bare electrode (in black) and the four modification steps: addition of glutaraldehyde (in red), functionalization of ACE2 (in blue), addition of the blocking agent bovine serum albumin (in green), and addition of the Nafion permselective membrane (in purple). FIG. 26D provides Nyquist plots for different SP concentrations ranging from 100 fg mL-1 to 100 ng mL-1 with 10-fold increments in neat saliva from a healthy donor (negative result by RT-qPCR). The inset shows the linearized correlation between normalized R_{CT} values and the concentration of SP exposed to the electrode. FIG. 26E provides Nyquist plots for tittered inactivated virus solutions at concentrations ranging from 101 to 106 PFU mL-1 with 10-fold increments. The upper left inset shows the linearized correlation between the normalized R_{CT} values and concentration of inactivated virus in solution. The lower right inset shows a zoomed region of the curve with the Nyquist plots' semi-arc (R_{CT}). The analytical curves presented in FIGS. 26D and 26C were based on triplicate measurements. All data were recorded using the eCHIP version of DETECT.

We designed the electrochemical device to explore the remarkable binding affinity of SARS-CoV-2 spike protein (SP) to ACE2, its receptor in the human body (Andersen et al., 2020; Yang et al., 2020) (FIG. 26A). The working electrode (WE), where the (electro)chemical reaction/interaction takes place and is converted to a detectable analytical signal, was functionalized by a drop-casting method. Enzyme immobilization was achieved by cross-linking ACE2 using the bifunctional chemical cross-linker glutaraldehyde (GA) (Barbosa et al., 2014). This dialdehyde reacts mainly with the primary amino groups of proteins, for example, the ε-amino group of lysine residues or the N-terminal group of the protein chain (Pereira et al., 2018). We used bovine serum albumin (BSA) to block the electrode's surface after immobilization of ACE2. BSA is a functionally inert protein with a high density of superficial lysine residues that is commonly used for biosensor development (Pereira et al., 2018).

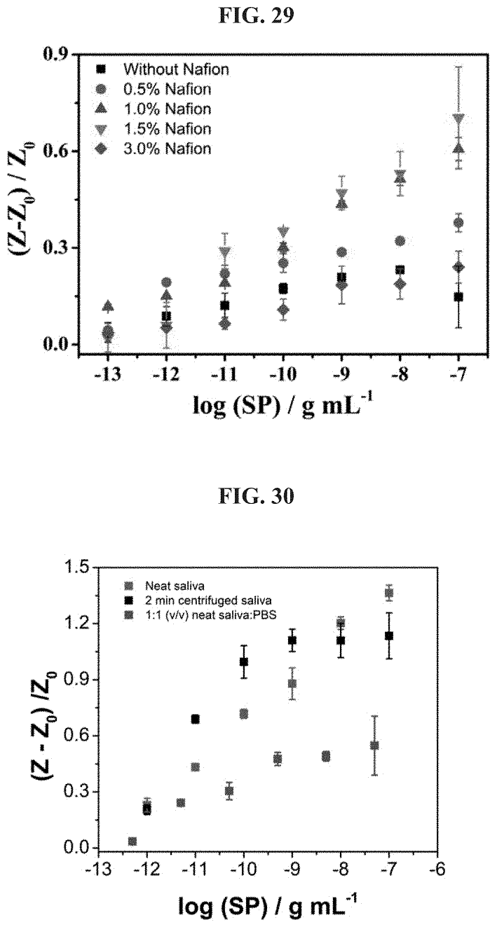

Using these well-established protocols for bioelectrode development, we first added GA for 1 hour at 37° C. to fully cover the carbon electrode surface generating a cross-linked polymer that enables the covalent anchoring of ACE2 at 37° C. for 1.5 hours (FIG. 25B). Next, BSA was added to the surface of the electrode for 30 minutes at 37° C. to block possible remaining active sites (i.e., working electrode's surface areas that were not functionalized with ACE2) thus preventing nonspecific adsorption to the GA layer by other proteins. We also incorporated an additional functionalization step using a 1.0% Nafion solution (FIG. 29) to create a protective polymeric membrane enhancing the robustness of the biosensor (Mauritz and Moore, 2004). Interestingly, Nafion increased up to 2-fold the sensitivity of the biosensor, particularly when used at a concentration ranging between 1.0% and 1.5% (FIG. 29). Given these results, we selected 1.0% Nafion (wt %) for subsequent optimization steps because of its optimal analytical response to low reagent usage ratio (Mauritz and Moore, 2004). This anionic membrane enables small positively charged species to cross and preconcentrate close to the biosensing surface. The Nafion layer also enhanced the robustness of DETECT by protecting against biofouling of the electronic surface when exposed to the sample's complex matrix (e.g., proteins, lipids, and other macromolecules present in biological samples) that may interfere with the detection (e Silva et al., 2020; Mauritz and Moore, 2004)

The optimized protocol generated the best analytical signal for the detection of SARS-CoV-2 in human biofluid samples (FIG. 25A). It consists of the following 4-steps: 1) modifying the working electrode with the immobilizing agent (GA); 2) covalent attachment of the recognition agent ACE2; 3) addition of the stabilization and active site blocking agent BSA; and 4) incorporating the permselective membrane (Nafion). A detailed protocol describing biosensor preparation, including the production of the screen-printed devices and functionalization, is provided in the Examples, infra.

Our test can be performed at room temperature with minimal equipment and reagents, and costs $4.67 to produce [$0.07 to produce the bare electrode, $4.50 to functionalize the electrode with the recognition agent ACE2, and $0.10 to coat the electrode with GA, BSA, and Nafion used (FIG. 19C)]. The overall cost of DETECT may be further reduced through recombinant production of ACE2 and ACE2 variants (Chan et al., 2020). Our technology is also highly scalable, as the electrodes can be rapidly mass-produced by using commercially available screen-printers. One laboratory-sized unit is able to produce 35,000 electrodes daily (1.05 M electrodes/month) and this could scaled-up to 10.5 billion electrodes monthly with only 10,000 screen-printers (Table 1′). These estimates take into account both the time needed to print the electrodes and all functionalization steps (i.e., 1 hour for GA functionalization, 1.5 hours to incorporate ACE2, 0.5 hours for BSA, and 1 hour for Nafion; total of 4 hours). However, it must be noted that these steps can be fully automated into a production line for industrial purposes, drastically reducing time requirements.

TABLE 1
DETECT 1.0: a scalable technology. Scalability of the
production of electrodes over a one-year period with laboratory screen-printers and industrial
screen-printers. The numbers shown reflect both the number of printed electrodes over time
considering the printing rate of the screen printer and all functionalization steps (addition of
the anchoring agent, anchoring the recognition agent, addition of the blocking agent, and
generation of the perm-selective membrane, the latter of which may take 4 additional hours
after electrodes are printed.

| | Number of electrodes produced | | | | | |
|---|---|---|---|---|---|---|
| | | | | 1 Industrial | 100 Industrial | 10,000 Industrial |
| Time | 1 Screen- | 100 Screen- | 10,000 Screen- | Screen- | Screen- | Screen- |
| (days) | printer | printers | printers | printer | printers | printers |
| 7 | 245,000 | 24,500,000 | 2,450,000,000 | 1,050,000 | 105,000,000 | 10,500,000,000 |
| 15 | 525,000 | 52,500,000 | 5,250,000,000 | 2,250,000 | 225,000,000 | 22,500,000,000 |
| 30 | 1,050,000 | 105,000,000 | 10,500,000,000 | 4,500,000 | 450,000,000 | 45,000,000,000 |
| 365 | 12,775,000 | 1,277,500,000 | 127,750,000,000 | 54,750,000 | 5,475,000,000 | 547,500,000,000 |

The key steps required for the electrode's functionalization were optimized and characterized (FIGS. 26B-C). Additionally, we evaluated the incubation time (i.e. time of exposure of the sample to the biosensor to enable sensitive detection), and whether a centrifugation/dilution step was needed to detect SARS-CoV-2 in complex biological samples such as saliva⁸. These optimization steps revealed that an additional centrifugation step was not needed (FIG. 30) since the use of neat saliva yielded similar results to those obtained using centrifuged samples. FIG. 30 provides calibration curves of the SP ranging from 500 fg mL⁻¹ to 100 ng mL⁻¹, where the saliva samples were incubated using three different setups: (i) direct use, i.e., without any pretreatment; (ii) neat saliva after 2 min of centrifugation at 10,000 rpm; and (iii) after simple 1:1 dilution in PBS. We can observe that the use of neat saliva allows the same detection efficacy and greater linear behavior when compared to the other pretreatment conditions. All measurements were recorded in triplicate using eChips.

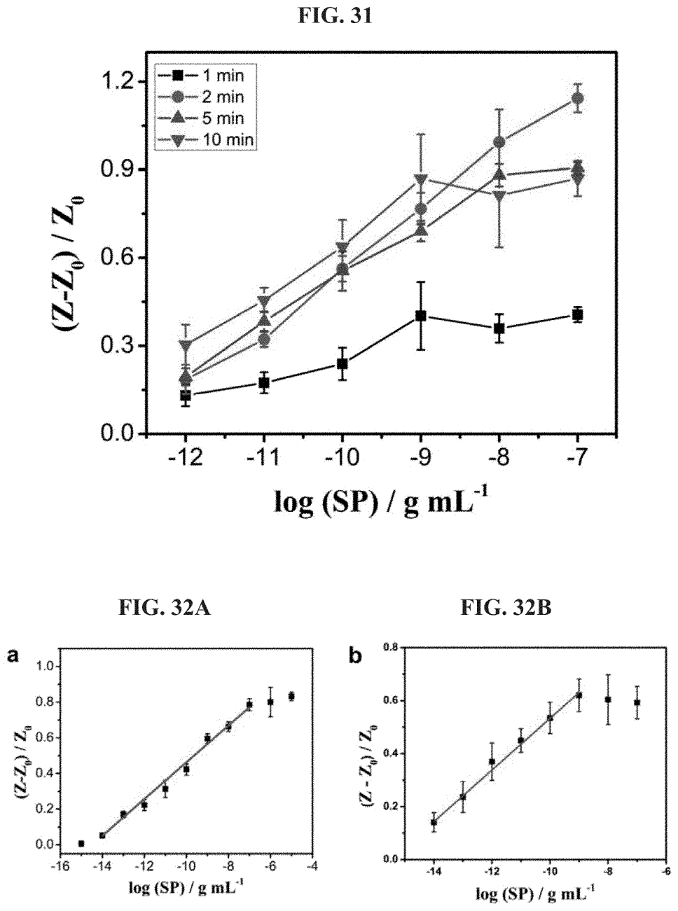

These results demonstrated that our approach is robust and can directly use human samples (NP/OP or saliva) without a prior pretreatment step, thus allowing the application of DETECT for streamlined and rapid point-of-care diagnosis. We selected 2 minutes as the optimal incubation period of the sample on the working electrode's surface for sensitive SARS-CoV-2 detection in samples considering the detectability and analytical frequency of the tests (FIG. 31). Our very minimal incubation time requirement (2 minutes) confirms the favorable configuration of the modified electrode that allows rapid interaction kinetics between the SP and immobilized ACE2 [kinetics constant rate of 10⁴M⁻¹s⁻¹ in its natural environment (Yang et al., 2020)]. Overall, DETECT provides a result in 4 minutes (2 minutes of sample incubation+2 minutes to perform the EIS analysis), which is vastly faster than methods currently available for diagnosing COVID-19 (FIG. 25C). It is important to note that the total time required to run each blank is an additional 4 minutes. However, we did not take this into account in our testing time calculations because the blanking step can be done before analyzing clinical samples, and we can use the R_{CT} values obtained for the blanks (PBS or VTM) to compare with the patient sample values.

Taking into account the optimal analytical conditions evaluated (Table 1′), we built calibration curves for free SP (FIGS. 26D, 32A, 32B) and heat-inactivated virus using the normalized R_{CT} response, defined by the following equation:

$$normalized {R}_{CT} = \frac{Z - {Z}_{0}}{{Z}_{0}}$$

where Z is the R_{CT} of the sample and Z₀ is the R_{CT} of the respective blank solution: phosphate buffer saline (PBS), virus transportation medium (VTM), or healthy saliva. The normalization process of R_{CT} aims to correct eventual fluctuations in the sensor operation, such as the temperature at the testing point or variations due to analyst operation.

TABLE 2′
Analytical parameters of DETECT 1.0.

| Parameter | Value | |
|---|---|---|
| Linear concentration range (SP in PBS) | 10 fg mL⁻¹-100 ng mL⁻¹ | |
| Linear concentration range (SP in VTM) | 10 fg mL⁻¹-1 ng mL⁻¹ | |
| Linear concentration range (SP in saliva) | 100 fg mL⁻¹-100 ng mL⁻¹ | |
| Limit of detection (SP in PBS) | 2.18 | fg mL⁻¹ |
| Limit of detection (SP in VTM) | 6.29 | fg mL⁻¹ |
| Limit of detection (SP in saliva) | 1.39 | pg mL⁻¹ |
| Limit of quantification (SP in PBS) | 7.26 | fg mL⁻¹ |
| Limit of quantification (SP in VTM) | 20.96 | fg mL⁻¹ |
| Limit of quantification (SP in saliva) | 4.63 | pg mL⁻¹ |
| Working concentration range (IV in VTM) | 10¹-10⁶ PFU mL⁻¹ | |
| Limit of detection (IV in VTM) | 1.16 | PFU mL⁻¹ |
| Limit of quantification (IV in VTM) | 3.87 | PFU mL⁻¹ |

The dose-response curve for the free SP in PBS solution ranged from 1 fg mL⁻¹ to 10 μg mL¹ (FIG. 32A). A linear concentration range from 10 fg mL⁻¹ to 100 ng mL⁻¹ was obtained (R²=0.993) and limits of detection (LOD) and quantification (LOQ) were calculated as 2.18 fg mL⁻¹ and 7.26 fg mL⁻¹ SP based on signal to noise ratios (S/N=3) and (S/N=10), respectively. We built a dose-response for the free SP in VTM medium at a concentration range from 10 fg mL⁻¹ to 100 pg mL⁻¹ (FIG. 32B). A linear concentration range from 10 fg mL⁻¹ to 1 ng mL⁻¹ was obtained (R²=0.995) and limits of detection (LOD) and quantification (LOQ) were calculated as 6.29 fg mL⁻¹ and 20.96 fg mL⁻¹ SP based on the signal to noise ratio (S/N=3) and (S/N=10), respectively. When performed in neat saliva, the calibration curve was built at a concentration ranging from 100 fg mL⁻¹ to 100 ng mL⁻¹ (FIG. 26D). The calculated LOD and LOQ were 1.39 pg mL⁻¹ and 4.63 pg mL⁻¹, respectively. The higher LODs obtained in saliva and VTM are consistent with the increased sample complexity compared to PBS solution.

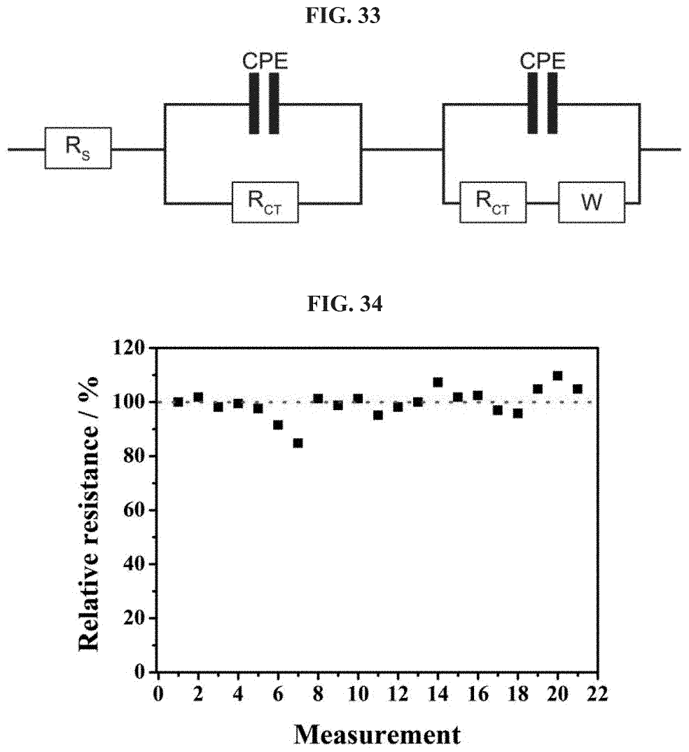

The R_{CT} values of Nyquist plots were extracted by the application of an equivalent circuit (FIG. 33). The equivalent circuit comprises two semi-arc regions observed in the Nyquist plots, where the first is a non-defined semi-arc at a high-frequency range due to inhomogeneity or defects in the electrode modification step (during drop-casting functionalization) and considerably small (R_{CT}˜10Ω) (Bertok et al., 2019; Uygun and Ertu\check{g}rul Uygun, 2014). The second parallel component of the equivalent circuit comprises an R_{CT}, whose signal intensity was proportional to the logarithm of the SP/virus concentration, and also presented a Warburg element to describe the mass transport (diffusional control).

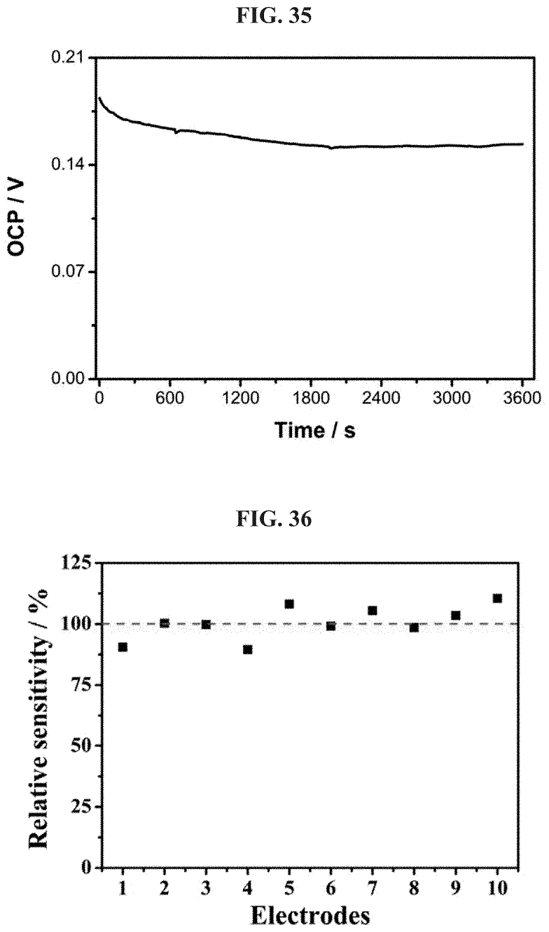

The concentration range of SP detected by our device was 10-1,000 times lower than that reported in previous studies (Rashed et al., 2021; Seo et al., 2020), thus underscoring the sensitivity of our approach. To assess the diagnostic capability of DETECT, we calibrated our biosensor using tittered solutions of inactivated SARS-CoV-2 ranging from 10¹ to 10⁶ PFU mL⁻¹. DETECT exhibited high sensitivity presenting a limit of detection (LOD) of 1.16 PFU mL⁻¹, which corresponds to the order of 10° RNA copies μL⁻¹ (Rao et al., 2020; Uhteg et al., 2020), a viral load that correlates with the initial stages of COVID-19 (i.e., 2 to 3 days after onset of symptoms)(Zou et al., 2020). Thus, DETECT's LOD and LOQ values are comparable to those of gold-standard approaches such as RealStar® SARS-CoV-2, CDC COVID-19, and e-Plex® SARS-CoV-2 (Uhteg et al., 2020) with the advantage of detecting symptomatic and asymptomatic individuals at the earliest stages of the infection allowing for rapid decision-making and the subsequent use of more appropriate and effective countermeasures. To ensure the repeatability, stability, and reproducibility of the results, we carried out three different experiments. First, 21 successive EIS measurements of the medium (PBS) were performed using the same device to verify the drift of the EIS response, yielding an RSD value of 5.3% (FIG. 34). These results demonstrated that the device exhibits a repeatable and stable response. Next, a measurement of open circuit potential before and after the addition of 1.0 ng mL⁻¹ of SP in PBS was recorded for 60 minutes (FIG. 35) and a small change in the potential (RSD value of 0.76%) was observed during the 30 minutes of exposure to 1.0 ng mL⁻¹ of SP solution. Finally, the reproducibility of DETECT was evaluated by analytical curves in the range of 1 pg mL⁻¹ to 1 ng mL⁻¹ of SP and the analytical sensitivity of 10 electrodes from different batches was assessed (FIG. 36). An RSD value of 6.8% was obtained, indicating that the electrochemical device fabrication and functionalization protocols display high reproducibility.

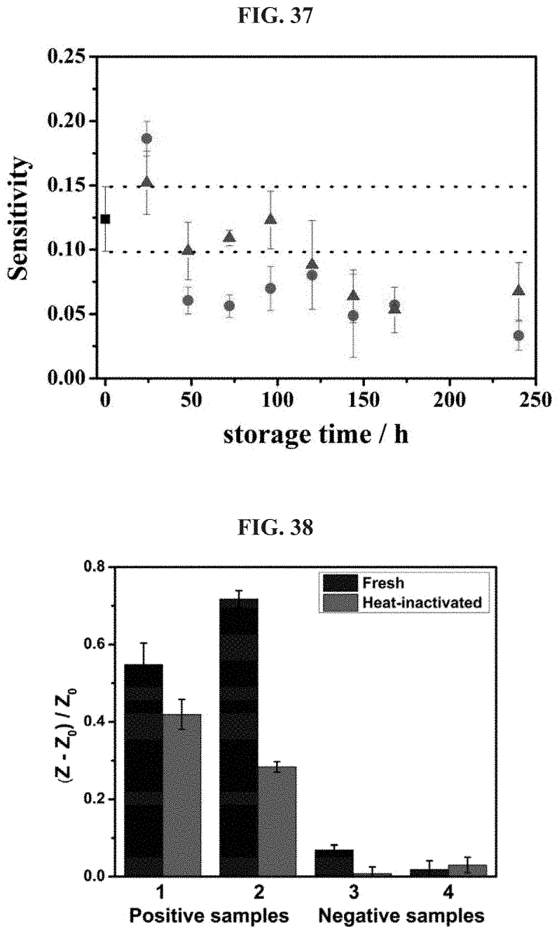

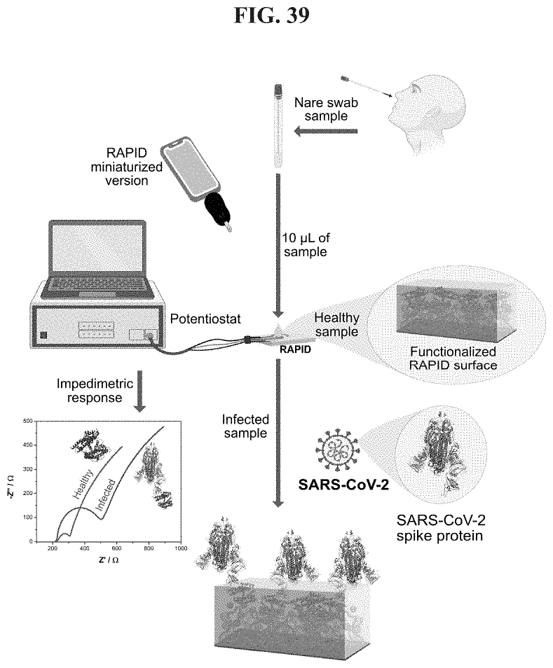

Next, we evaluated the stability of DETECT at different temperature storage conditions (25° C., 8° C., and −20° C.) over 10 days (FIG. 37). Analytical curves were generated with SP at a concentration ranging from 1 pg mL⁻¹ to 1 ng mL⁻¹ and the sensitivity was normalized by the mean value of the three different biosensors used immediately after the functionalization steps. The biosensors stored at room temperature did not detect the SP after 24 hours due to loss of enzymatic activity (FIG. 37). The sensors stored at 8° C. were stable after 24 hours, but after 48 hours presented decreased sensitivity (around 50% of the initial response) keeping this low sensitivity for 7 days (FIG. 37). Biosensors stored at −20° C. exhibited the most promising results since they were as sensitive as those used right after functionalization even after 96 hours and retained 50% of their sensitivity after 10 days of storage (FIG. 37).

Next, the performance of DETECT was assessed using both SARS-CoV-2-positive and negative clinical samples from the Hospital of the University of Pennsylvania (HUP) (Tables 3′ and 5′, below), including a highly contagious SARS-CoV-2 UK B.1.1.7 variant (Tables 3′ and 4′, below). All samples were heat-inactivated at 56° C. for 1 hour. The effect of heat inactivation of SARS-CoV-2 samples on the analytical response of our biosensor was evaluated through measurements taken before and after sample inactivation at 56° C. for 1 hour (FIG. 38). The results indicated that thermal inactivation affected the ability of SP to bind to ACE2, since a decrease of up to 60% was detected in the analytical response for sample 2 after heat inactivation (FIG. 38). These results indicate that heat-inactivated clinical samples with very low viral titers may fall below our current limit of detection. Rath and Kumar (Rath and Kumar, 2020) demonstrated using molecular dynamics simulations that temperatures >50° C. trigger the closing of the spike receptor binding motif (RBM), which buries the receptor binding residues preventing contacts between the SP and the ACE2 receptor. These insights may help explain the results obtained upon thermal inactivation of our biosensor (FIG. 38). However, despite this decrease in SP binding to ACE2 upon heat inactivation, the sensitivity of our method still enabled accurate viral detection in clinical samples containing a range of viral titers (FIG. 26E).

We also observed that centrifuging the samples did not lead to increased impedimetric detection of the SP (FIG. 30). Therefore, the NP/OP and saliva samples were used in VTM and PBS, respectively, following the Food and Drug Administration (FDA) recommendation for regulatory applications. Of note, the detectability of impedimetric measurements after 2 minutes of incubation of the sample on the working electrode's surface was as high as longer incubation times of 5 and 10 minutes (FIG. 31), thus demonstrating DETECT's fast interaction kinetics between the SP and functionalized WE, as discussed above. Thus, we selected 2 minutes of incubation and set as a cut-off value a 10% change in the R_{CT} when compared to the blank solution. Such a cut-off threshold takes into account the LOQ obtained for inactivated virus analysis (FIG. 26E), thus allowing discrimination between SARS-CoV-2 negative and SARS-CoV-2 positive samples (Tables 3′ and 5′, below).

In blinded tests, we analyzed 139 NP/OP swab samples (in VTM) obtained from patients after heat-inactivation, 109 of which were COVID-19 positive and 30 COVID-19 negative as determined by RT-qPCR and clinical assessment (Table 3, below). DETECT demonstrated high sensitivity, specificity and accuracy for NP/OP (83.5%, 100% and 87.1%, respectively; Table 4′) and saliva (100%, 86.5% and 90.0%, respectively; Table 4′) samples. DETECT missed a single sample, which presented a viral count lower than its LOD (10 RNA copies μL⁻¹). It is worth noting that although the heat inactivation protocol decreased the response of our biosensor due to the inactivation of SP (FIG. 38), the outstanding sensitivity of DETECT (Table 2′) enabled high detectability (Table 2′) and hit rate (Table 4′, below). Out of the 12 negative NP/OP swab samples present in our sample set, 100% were confirmed as SARS-CoV-2 negative by DETECT (data not shown). In addition, the highly contagious SARS-CoV-2 UK variant B.1.1.7 was obtained from a government testing site in Philadelphia (Tables 4′ and 5′, below). DETECT successfully identified this sample as positive with a normalized R_{CT}. value of 1.10 (Table 3′), thus underscoring its ability to detect emerging mutant variants of SARS-CoV-2.

TABLE 3′
Diagnosis of NP/OP samples from patients of the
Hospital of the University of Pennsylvania (HUP)
with COVID-19 symptoms using DETECT 1.0.

| | COVID-19 | | DETECT | |
|---|---|---|---|---|
| NP/OP Sample ID | Status | RT-qPCR | 1.0 | R_{CT} |
| 257 | + | + | − | 0 |
| 312 | + | + | + | 0.177 |
| 357 | − | − | − | 0.024 |
| 255 | − | − | − | 0.013 |
| 307 | + | + | + | 0.161 |
| Mock-1 | − | − | − | 0 |
| 312 | + | + | + | 0.159 |
| 356 | − | − | − | 0.021 |
| 263 | + | + | + | 0.149 |
| 290 | + | + | + | 0.123 |
| 314 | + | + | + | 0.263 |
| 256 | + | + | + | 0.210 |
| 334 | + | + | + | 0.254 |
| 257 | + | + | + | 0.155 |
| 251 | + | + | + | 0.171 |
| 309 | + | + | + | 0.136 |
| 332 | − | − | − | 0.029 |
| 336 | + | + | + | 0.16 |
| 290 | + | + | + | 0.145 |
| Mock-2 | − | − | − | 0 |
| 353 | − | − | − | 0 |
| Mock-3 | − | − | − | 0 |
| 262 | + | + | + | 0.118 |
| 360 | − | − | − | 0 |
| 348 | + | + | + | 0.261 |
| 358 | | | | 0 |
| 363 | − | − | − | 0.080 |
| 346 | + | + | + | 0.128 |
| 348 | + | + | + | 0.168 |
| 361 | − | − | − | 0.0722 |
| UPHS COVID 1 | + | + | + | 0.275 |
| UPHS COVID 4 | + | + | − | 0 |
| UPHS COVID 5 | + | + | + | 0.255 |
| UPHS COVID 6 | + | + | + | 0.200 |
| UPHS COVID 7 | + | + | + | 0.106 |
| UPHS COVID 8 | + | + | − | 0.098 |
| UPHS COVID 9 | + | + | + | 0.109 |
| UPHS COVID 10 | + | + | + | 0.272 |
| UPHS COVID 11 | + | + | − | 0 |
| UPHS COVID 12 | + | + | − | 0 |
| UPHS COVID 13 | + | + | − | 0 |
| UPHS COVID 14 | + | + | + | 0.167 |
| UPHS COVID 15 | + | + | + | 0.114 |
| UPHS COVID 16 | + | + | + | 0.137 |
| UPHS COVID 17 | + | + | + | 0.143 |
| UPHS COVID 18 | + | + | + | 0.241 |
| UPHS COVID 19 | + | + | + | 0.241 |
| UPHS COVID 20 | + | + | + | 1.011 |
| UPHS COVID 21 | + | + | + | 1.082 |
| UPHS COVID 22 | + | + | + | 0.183 |
| UPHS COVID 23 | + | + | + | 0.725 |
| UPHS COVID 24 | + | + | + | 0.107 |
| UPHS COVID 25 | + | + | + | 0.175 |
| UPHS COVID 26 | + | + | + | 0.104 |
| UPHS COVID 27 | + | + | + | 0.110 |
| UPHS COVID 28 | + | + | + | 0.171 |
| UPHS COVID 32 | + | + | + | 0.129 |
| UPHS COVID 35 | + | + | + | 0.191 |
| UPHS COVID 36 | + | + | + | 0.745 |
| UPHS COVID 37 | + | + | + | 0.110 |
| UPHS COVID 39 | + | + | + | 0.130 |
| UPHS COVID 40 | + | + | + | 0.261 |
| UPHS COVID 42 | + | + | + | 0.108 |
| UPHS COVID 44 | + | + | + | 0.179 |
| UPHS COVID 45 | + | + | − | 0.024 |
| UPHS COVID 46 | + | + | + | 0.114 |
| UPHS COVID 47 | + | + | + | 0.103 |
| UPHS COVID 48 | + | + | + | 0.146 |
| UPHS COVID 49 | + | + | + | 0.101 |
| UPHS COVID 50 | + | + | + | 0.126 |
| UPHS COVID 51 | + | + | + | 0.132 |
| UPHS COVID 52 | + | + | + | 0.131 |
| UPHS COVID 53 | + | + | + | 0.191 |
| UPHS COVID 54 | + | + | + | 0.107 |
| UPHS COVID 55 | + | + | + | 0.225 |
| UPHS COVID 56 | + | + | + | 0.104 |
| UPHS COVID 57 | + | + | + | 0.188 |
| UPHS COVID 58 | + | + | + | 0.194 |
| UPHS COVID 59 | + | + | − | 0 |
| UPHS COVID 60 | + | + | − | 0 |
| UPHS COVID 61 | + | + | − | 0 |
| UPHS COVID 62 | + | + | + | 0.139 |
| UPHS COVID 63 | + | + | + | 0.229 |
| UPHS COVID 64 | + | + | + | 0.481 |
| UPHS COVID 65 | + | + | − | 0 |
| UPHS COVID 66 | + | + | + | 0.152 |
| UPHS COVID 67 | + | + | + | 0.197 |
| UPHS COVID 68 | + | + | + | 0.105 |
| UPHS COVID 69 | + | + | + | 0.101 |
| UPHS COVID 70 | + | + | + | 0.129 |
| UPHS COVID 71 | + | + | + | 0.101 |
| UPHS COVID 72 | + | + | − | 0 |
| UPHS COVID 73 | + | + | + | 0.159 |
| UPHS COVID 74 | + | + | − | 0.003 |
| UPHS COVID 75 | + | + | + | 0.222 |
| UPHS COVID 76 | + | + | + | 0.101 |
| UPHS COVID 77 | + | + | + | 0.236 |
| UPHS COVID 78 | + | + | + | 0.342 |
| UPHS COVID 79 | + | + | − | 0.012 |
| UPHS COVID 80 | + | + | + | 0.102 |
| UPHS COVID 81 | + | + | − | 0.031 |
| UPHS COVID 82 | + | + | + | 0.302 |
| UPHS COVID 83 | + | + | + | 0.127 |
| UPHS COVID 84 | + | + | + | 0.165 |
| UPHS COVID 85 | + | + | + | 0.130 |
| UPHS COVID 86 | + | + | + | 0.102 |
| UPHS COVID 87 | + | + | + | 0.221 |
| UPHS COVID 88 | + | + | + | 0.196 |
| UPHS COVID 89 | + | + | − | 0.035 |
| UPHS COVID 90 | + | + | + | 0.137 |
| UPHS COVID 91 | + | + | − | 0 |
| UPHS COVID 92 | + | + | + | 0.13 |
| UPHS COVID 93 | + | + | + | 0.184 |
| UPHS COVID 94 | + | + | − | 0 |
| UPHS COVID 95 | + | + | + | 0.115 |
| UPHS COVID 96 | + | + | + | 0.101 |
| UPHS COVID 97 | + | + | + | 0.236 |
| UPHS COVID 98 | + | + | + | 0.630 |
| UPHS COVID 99 | + | + | + | 0.582 |
| UPHS COVID 100 | + | + | + | 0.102 |
| 700067571 | − | − | − | 0.05 |
| 601112 | − | − | − | 0 |
| 466721776 | − | − | − | 0 |
| 633400 | − | − | − | 0.06 |
| 349368993 | − | − | − | 0.02 |
| 442134375 | − | − | − | 0 |
| 468444690 | − | − | − | 0.07 |
| 346496821 | − | − | − | 0.073 |
| 357098938 | − | − | − | 0 |
| 440956795 | − | − | − | 0 |
| 363618695 | − | − | − | 0 |
| 042 | − | − | − | 0 |
| 044 | − | − | − | 0.028 |
| 046 | − | − | − | 0.078 |
| 047 | − | − | − | 0.044 |
| 049 | − | − | − | 0.09 |
| 053 | − | − | − | 0.077 |
| 054 | − | − | − | 0.077 |
| 100667644* | + | + | + | 1.098 |

*Individual infected with a highly contagious SARS-CoV-2 UK variant B.1.1.7.

DETECT demonstrated high sensitivity, specificity and accuracy (96.2%, 100% and 97.4%, respectively; Table 4′).

TABLE 4
Positive and negative values obtained by RT-qPCR, and sensitivity, specificity, and
accuracy of DETECT 1.0 using NP/OP and saliva samples.

| | RT-qPCR | | | | | |
|---|---|---|---|---|---|---|
| DETECT | Positive | Negative | Total | | | |
| (NP/OP) | (N = 109*) | (N = 30) | (N = 139) | Sensitivity | Specificity | Accuracy |
| Positive | 91 | 0 | 91 | 91/109 | | |
| | | | | (83.5%) | | |
| Negative | 18 | 30 | 48 | | 30/30 | 121/139 |
| | | | | | (100%) | (87.1%) |
| | RT-qPCR | | | | | |
| DETECT | Positive | Negative | Total | | | |
| (Saliva) | (N = 13) | (N = 37) | (N = 50) | Sensitivity | Specificity | Accuracy |
| Positive | 13 | 5 | 18 | 13/13 | | |
| | | | | (100.0%) | | |
| Negative | 0 | 32 | 32 | | 32/37 | 45/50 |
| | | | | | (86.5%) | (90.0%) |

*Clinical sample set includes a highly contagious SARS-CoV-2 UK variant B.1.1.7 from a patient.

To evaluate DETECT's diagnostic efficacy in a more complex biological environment, we tested saliva samples from 50 patients (Table 5′) under the same conditions used for the NP/OP swab samples.

TABLE 5′
Diagnosis of saliva samples from patients of the
Hospital of the University of Pennsylvania (HUP)
with COVID-19 symptoms using DETECT 1.0.

| ED Saliva | COVID-19 | | DETECT | |
|---|---|---|---|---|
| Sample ID | Status | RT-qPCR | 1.0 | R_{CT} |
| 1 | − | − | − | 0 |
| 2 | − | − | − | 0 |
| 3 | + | + | + | 0.261 |
| 4 | − | − | − | 0.099 |
| 6 | + | + | + | 0.573 |
| 9 | − | − | − | 0 |
| 14 | − | + | + | 0.252 |
| 21 | − | + | + | 0.121 |
| 24 | − | − | − | 0.050 |
| 33 | + | + | + | 0.303 |
| 41 | − | − | − | 0.069 |
| 42 | + | − | + | 0.751 |
| 43 | − | + | + | 0.154 |
| 44 | − | − | − | 0.076 |
| 45 | − | + | + | 0.176 |
| 46 | − | − | − | 0.096 |
| 51 | − | − | − | 0 |
| 52 | − | − | − | 0 |
| 53 | − | − | − | 0 |
| 54 | − | − | − | 0 |
| 55 | − | − | − | 0.035 |
| 56 | − | − | + | 0.232 |
| 58 | + | + | + | 0.223 |
| 69 | − | − | − | 0.081 |
| 70 | + | + | + | 1.103 |
| 72 | | | | 0.083 |
| 77 | + | + | + | 0.181 |
| 79 | − | − | − | 0.012 |
| 82 | + | + | + | 0.302 |
| 90 | + | + | + | 0.137 |
| 91 | + | + | + | 0.132 |
| 700067571 | − | − | − | 0.08 |
| 453299679 | − | − | − | 0 |
| 468349915 | − | − | − | 0.03 |
| 633400 | − | − | − | 0 |
| 349368993 | − | − | − | 0 |
| 464333574 | + | + | + | 0.134 |
| 468444690 | − | − | − | 0.08 |
| 335835294 | + | + | + | 0.102 |
| 346496821 | − | − | − | 0.072 |
| 357098938 | − | − | − | 0 |
| 440956795 | − | − | − | 0 |
| 363618695 | − | − | − | 0 |
| 041 | − | − | − | 0 |
| 042 | − | − | − | 0 |
| 043 | − | − | − | 0 |
| 044 | − | − | − | 0 |
| 046 | − | − | − | 0 |
| 047 | − | − | − | 0 |
| 096 | + | + | + | 0.293 |

The greater complexity of saliva, compared to swab samples, is known to hinder the accurate detection of infectious agents (Jamal et al., 2020; Zou et al., 2020). Saliva is a biofluid that is susceptible to large variations in composition depending on different factors such as the ingestion of food and drinks prior (30-60 minutes) to sample collection, which can lead to the dilution of the saliva matrix, and the insertion of exogenous molecular species that may interfere with accurate detection. Even using highly heterogenous saliva samples, the sensitivity of DETECT remained high (100%), however false positives led to decreased specificity (86.5%), and an accuracy of 90.0% (Table 4′). The latter results may be explained by potential interactions between ACE2, which is a carboxypeptidase and amino acid transporter, and other biomolecules that can be found in neat biofluids, such as regulatory peptides and peptide hormones (e.g., angiotensin, bradykinin, ghrelin, apelin, neurotensin, and dynorphin) (Turner, 2015). Thus, we believe the performance of DETECT will improve when using fresh saliva samples at the point-of-care. It is worth noting that among the SARS-CoV-2-positive saliva samples, our test identified as positive two samples that had been previously erroneously detected as negative by RT-qPCR, therefore indicating that DETECT may help correctly diagnose COVID-19 in samples previously misdiagnosed by other methods.

Several key analytical features were used to compare the performance of DETECT with respect to other electrochemical methods reported in the literature (Table 6′).

TABLE 6
Comparison of methods reported to COVID-19 diagnosis.

| | | | Lowest | Number of | | | |
|---|---|---|---|---|---|---|---|
| | | Biological | Concentration | Clinical | Price | Time | |
| Sensor | Technique | Target | Detected | Samples | (US$) | (min) | Reference |
| DETECT | EIS | SARS- | 2.8 fg mL⁻¹ | 151 | 4.67 | 4 | This work |
| 1.0 | | CoV-2 | | | | | |
| | | spike | | | | | |
| | | protein | | | | | |
| SARS- | DPV and | Viral | 500 pg mL⁻¹ | 16 | — | 10 | (Torrente- |
| CoV-2 | OCP-EIS | antigen | | | | | Rodriguez et |
| RapidFlex | | nucleocapsid | | | | | al., 2020) |
| | | protein | | | | | |
| SARS- | DPV and | IgM and | 250 ng mL⁻¹ | 16 | — | 10 | (Torrente- |
| CoV-2 | OCP-EIS | IgG | | | | | Rodriguez et |
| RapidFlex | | antibodies | | | | | al., 2020) |
| SARS- | DPV and | C-reactive | 50 ng mL⁻¹ | 16 | — | 10 | (Torrente- |
| CoV-2 | OCP-EIS | protein | | | | | Rodriguez et |
| RapidFlex | | | | | | | al., 2020) |
| | SCC | SARS- | 231 RNA | 48 | 10 | 5 | (Alafeef et al., |
| | | CoV-2 | copies μL⁻¹ | | | | 2020) |
| | | RNA | | | | | |
| | DPV | SARS- | 200 RNA | 33 | — | <5 | (Zhao et al., |
| | | CoV-2 | copies μL⁻¹ | | | | 2021) |
| | | RNA | | | | | |
| | | SARS- | | | | | |
| | EIS | CoV-2 | 0.1 mg mL⁻¹ | 4 | — | 3 | (Rashed et al., |
| | | spike | | | | | 2021) |
| | | protein | | | | | |
| | SWV | IgM and | 1 μg mL⁻¹ | 17 | — | 45 | (Yakoh et al., |
| | | IgG | | | | | 2021) |
| | | antibodies | | | | | |
| DETECTR | CRISPR | E gene and | 10 RNA copies | 11 | — | 40 | (Broughton et |
| | technology | N gene | μL⁻¹ | | | | al., 2020) |
| | Colorimetric | N gene | 0.18 ng μL⁻¹ | 1 | — | 30 | (Moitra et al., |
| | assay | | | | | | 2020) |
| | Localized | RdRp | 2.26 × 10⁴ | 5 | — | 2 | (Qiu et al., |
| | surface | | RNA copies | | | | 2020) |
| | plasmon | | μL⁻¹ | | | | |
| | resonance | | | | | | |
| | DNA | Synthetic | 0.96 pmol L⁻¹ | 0 | — | 10 | Jiao et al., |
| | nanoscaffold- | RNA | | | | | 2020) |
| | based hybrid | conserved | | | | | |
| | chain reaction | region | | | | | |
| | RT-LAMP | orf1ab | 20-200 RNA | 130 | — | 60 | (Yan et al., |
| | | | copies μL⁻¹ | | | | 2020) |
| | RT-LAMP | N gene | 100 RNA | 27 | — | 30 | (Baek et al., |
| | | | copies μL⁻¹ | | | | 2020) |

EIS-Electrochemical impedance spectroscopy;
DPV-Differential pulse voltammetry;
OCP-EIS-Open-circuit potential-electrochemical impedance spectroscopy;
SCC-Signal conditioning circuit;
SWV-Square-wave voltammetry;
RT-LAMP-Reverse transcription loop-mediated isothermal amplification.

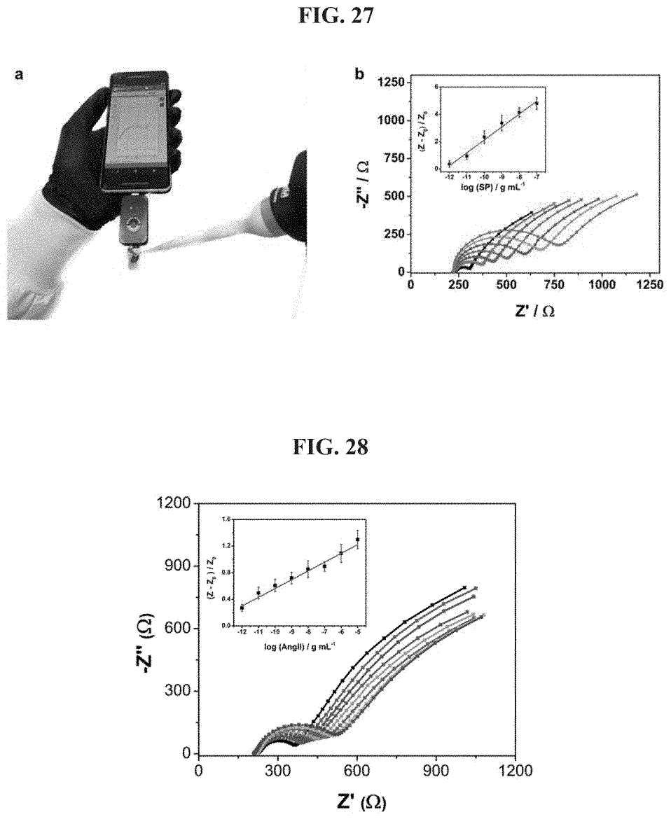

Our method provides the highest sensitivity (LOD of 2.8 fg mL⁻¹) for the detection of SARS-CoV-2 spike protein with excellent time of detection and overall cost (Table 6′). Additionally, the robustness of DETECT was evaluated in a large clinical sample set (Tables 3′ and 5′), and all results were compared with those obtained by RT-qPCR (Table 4′), thus highlighting the reliability of our method. All experiments described thus far (e.g., detection of SARS-CoV-2 spike protein and clinical samples) were performed using the eChip version of the electrode (e.g., FIG. 26, Table 4′, FIGS. 29 and 30). After successfully applying the eChip (composed of printed circuit board) to clinical samples (Tables 3′ and 5′) and obtaining robust and sensitive results (Table 2′), we sought to construct an optimized electrode composed of a material that was more accessible and inexpensive to enable scale-up production of DETECT. We selected filter paper as the main component of electrochemical paper-based analytical device (ePAD) as it is easy to handle (maleable), accessible, and inexpensive [paper filter costs $0.50 per 1 m² whereas printed circuit board (PCB) costs $40.00 per 1 m²] (Ataide et al., 2020; Ozer et al., 2020). We adapted and demonstrated the applicability of ePAD in a portable potentiostat connected to a smart device (FIG. 27A). We used the screen-printing method to fabricate the electrodes and combined wax-printing technology to pattern the electrochemical cell onto the paper filter. Thus, the ePAD is composed of more accessible and low-cost material, enabling scalable manufacturing and on-demand testing at the point-of-care (Ataide et al., 2020; Ozer et al., 2020).

To demonstrate the portability of DETECT and its potential as a point-of-care diagnostic test, we adapted and demonstrated its applicability in a portable potentiostat connected to a smart device. FIG. 27A and FIG. 27B illustrate the of the miniaturized and portable DETECT 1.0 for rapid point-of-care diagnosis of COVID-19. FIG. 27 represents an image of the mobile device-compatible handheld DETECT 1.0 during real-time sample analysis. FIG. 27B provides Nyquist plots obtained using ePAD coupled to a smart-device for different concentrations of SP ranging from 1 pg mL-1 to 100 ng mL-1. The inset shows the calibration curve for the normalized R_{CT} values of the different concentrations of SP.

In this case, a paper-based electrode (ePAD) was used, as this is a more accessible and low-cost material for onsite analysis. However, the cellulosic structure of the paper presents higher wettability compared to the phenolic circuit boards (eChip), causing the absorption of the sample by the electrode's paper surface. Therefore, in order to enhance the detectability (i.e., the LOD) of DETECT, we added 2.5-fold increased volumes of the modifiers (GA, ACE2, BSA, and Nafion) on the surface of the WE during the fabrication process. This approach allowed higher sensitivity towards the detection of SP, which was used to generate a calibration curve (FIG. 27B). We attribute the enhanced detection (7-fold increase) of the paper-based version of DETECT compared to the phenolic-based electrode (eChip) to the higher amount of recognition element (ACE2) used on the working electrode's surface. However, it is worth noting that the eChip version already demonstrated excellent performance at detecting SARS-CoV-2 (Tables 2′ and 4′) and, although its sensitivity can be further increased by using a higher concentration of ACE2 (FIG. 27B), this would increase the cost of the test since recombinant ACE2 accounts for 95% of the final cost of DETECT 1.0 (FIG. 25C).

DETECT diagnoses COVID-19 at its early stages compared to serological tests, which take 5-7 days to ensure reliable detection of IgG and IgM antibodies¹⁷. Our device presented higher accuracy, specificity, and selectivity than most existing methods available for SARS-CoV-2 detection¹¹. Our biosensor is inexpensive and portable, enabling decentralized diagnosis at the point-of-care. The time of detection of our approach (4 minutes) is significantly lower than existing diagnostic tests^{10,11,18}, and could potentially be lowered even more by using engineered versions of human ACE2 with enhanced selective binding towards SARS-CoV-2 SP′. The use of such ACE2 variants would also help reduce the rate of false positives in complex biofluids such as saliva^{7,19,20}.

DETECT presented higher accuracy, specificity, and selectivity than most existing electrochemical methods available for SARS-CoV-2 detection (Table 6′) (Uhteg et al., 2020). We also assessed DETECT's specificity in cross-reactivity assays by exposing our sensor to the following seven different viruses: three coronaviruses (MHV—murine hepatitis virus, HCoV-OC43—human coronavirus OC43, and human coronavirus 229E; Table S4) and four non-coronavirus viral strains (H1N1—A/California/2009, H3N2—A/Nicaragua, Influenza B—B/Colorado, HSV2—herpes simplex virus-2; Table 7′).

TABLE 7′
Cross-reactivity analysis of DETECT 1.0 when exposed
to other coronaviruses and non-coronavirus strains.

| ED Saliva Sample ID | DETECT 1.0 | R_{CT} |
|---|---|---|
| MHV | − | 0 |
| HCoV-OC43 | − | 0 |
| 229E | − | 0.06 |
| H1N1 | − | 0 |
| H3N2 | − | 0.04 |
| Influenza B | − | 0 |
| HSV2 | − | 0 |

We did not detect cross-reactivity events against any of the viruses tested (R_{CT}<10%) (Table 7′) thus further highlighting the translatability of our diagnostic test. Our biosensor is inexpensive and portable, enabling decentralized diagnosis at the point-of-care. The time of detection of our approach (4 minutes) is significantly lower than existing diagnostic tests (Kaushik et al., 2020; Rashed et al., 2021; Uhteg et al., 2020), and could potentially be lowered even more by using engineered versions of human ACE2 with enhanced selective binding towards SARS-CoV-2 SP (Chan et al., 2020). The use of such ACE2 variants would also help reduce the rate of false positives in complex biofluids such as saliva (Chan et al., 2020; Glasgow et al., 2020; Sorokina et al., 2020).

DETECT can also be multiplexed to allow detection of other emerging biological threats such as bacteria, fungi, and other viruses. Thus, our technology serves as a platform for the rapid diagnosis of COVID-19 and future endemic/pandemic outbreaks at the point-of-care. Its low cost, speed of detection, scalability, and implementation using smart devices and telemedicine platforms may facilitate much needed population-wide deployment.

### Additional Information Concerning Materials and Methods

The electrochemical sensors were screen-printed in a three-electrode configuration cell on two accessible substrates (i) a qualitative filter paper and (ii) phenolic paper circuit board material. Electrically conductive carbon and Ag/AgCl inks were used for the screen-printing process to fabricate the working/auxiliary electrodes and reference electrodes, respectively. The working electrode's carbon surface was modified using the drop casting method. First, 4 μL of 25% glutaraldehyde (GA) solution was added for 1 hour at 37° C. for the formation of a cross-linked polymer, which enabled the anchoring of ACE2 (4 μL at 0.32 mg mL⁻¹), then incubated at 37° C. for 1.5 hours. Next, 4 μL of bovine serum albumin (BSA) at 1 mg mL⁻¹ was added and the WE was allowed to dry for 0.5 hours at 37° C. to stabilize the enzyme through the co-reticulation and allow blockage of potential remaining active sites of the carbon electrode to avoid any nonspecific adsorption by other proteins to the glutaraldehyde layer and ensure stabilization of the ACE2 tertiary structure. Both concentrations of GA and BSA solutions were used in excess to ensure the complete functionalization and blocking of the WE's surface.

To test ACE2 conformational integrity after the addition of BSA to the functionalized electrode, the response of the electrode to angiotensin II, ACE2's natural substrate (FIG. 28) was analyzed. FIG. 28 provides Nyquist plots showing the response of the modified eChip to different concentrations of angiotensin II, the natural substrate of ACE2, ranging from 1 pg mL⁻¹ to 10 μg mL⁻¹. A sensitive linear response was observed in the range of 1 pg mL⁻¹ to 10 μg mL⁻¹ of angiotensin II, demonstrating that our anchoring and stabilization strategies maintained the functionality of ACE2's active sites and revealing that the biosensor architecture did not obstruct ACE2. The calibration curve was built based on triplicate measurements. The results showed that the anchoring and stabilization steps were effective on the WE's surface and there was no loss of ACE2's conformation integrity since it was able to interact with its natural substrate.

Since the objective was to simplify detection of SARS-CoV-2 in complex biological samples, such as neat saliva and NP/OP swabs, we added a 1% Nafion solution as an extra protective layer. Nafion solution, an anionic and permselective membrane, is commonly used to enhance the sensitivity and robustness of electrochemical sensors. In our study, the membrane formed by 1% Nafion solution enhanced the sensitivity of DETECT 1.0 (FIG. 29), by enabling chemical preconcentration of cation species and protecting the electrode's surface against biofouling of biomolecules present in biological samples, such as protein, lipids, and other macromolecules¹. FIG. 29 depicts calibration curves for the SP (ranging from 1 pg mL⁻¹ to 100 ng mL⁻¹) that were built with different Nafion concentrations (0, 1%, 3% and 5%; wt %) to test the effect of the permselective membrane on the analytical signal of DETECT 1.0. The optimal concentration of Nafion found was found to be 1% (wt %). It is worth noting that Nafion at 5% created a thicker film on the working electrode's surface that did not present high adherence to the surface and detached during the impedimetric measurements. Therefore, it was not possible to measure different concentrations of SP in solution.

The effect of each modifier layer on the electrochemical response of our modified electrode was characterized, recording cyclic voltammetry (CV) and EIS measurements in the presence of 5 mmol L⁻¹ potassium ferricyanide/ferrocyanide (the redox probe), FIGS. 20B and 20C. These results demonstrated that the peak current signal of the redox probe decreased when using CV and the resistance to charge transfer increased after each functionalization step. The decrease in the peak current signal occurs due to the addition of nonconductive materials (e.g., proteins) that block the active sites of the electronic surface, hindering the kinetics of charge transfer of the redox probe.

We next evaluated the stability of the biosensor by measuring 6 successive EIS measurements in undiluted healthy human saliva (negative result for COVID-19 by RT-qPCR) and the same sample spiked with 1 pg mL⁻¹ free SP. Relative standard deviations of 3.58% and 5.21% were obtained, respectively. These results demonstrate that the developed biosensor presents a very stable architecture and provide effective robustness for the detection of SP in complex sample.

We proceeded to analyze patient samples obtained from symptomatic patients at the Hospital of the University of Pennsylvania. We tested 35 NP/OP swabs (Table 3′) and 31 saliva samples (Table 5′) that were complementary confirmed as SARS-CoV-2 positive or SARS-CoV-2 negative by RT-qPCR.

Chemicals. All chemicals were of analytical grade and used without additional purification. Solutions were obtained by dissolving or diluting the reagents in appropriate electrolytes prepared in deionized water. Human angiotensin converting enzyme 2 (ACE2) was purchased from GenScript (USA), sulfuric acid, potassium chloride (KCl), potassium ferricyanide K3[Fe(CN)₆], potassium ferrocyanide K4[Fe(CN)₆], bovine serum albumin (BSA), Nafion (5%) and glutaraldehyde (25%) were obtained from Sigma Aldrich (USA), and phosphate buffer saline (PBS) solution was purchased from VWR (USA). Viral transport medium (VTM) was obtained from Thermo Fisher. Conductive carbon and Ag/AgCl inks were acquired from Creative Materials, USA. SARS-CoV-2 spike protein was kindly donated by Scott Hensley (University of Pennsylvania) and the inactivated samples were donated by Sara Cherry, Michael Feldman and Ronald Collman (University of Pennsylvania).

Fabrication of electrochemical devices. The electrochemical sensors were screen-printed in a three-electrode configuration cell (dimensions: 1.8×1.2 cm) on two accessible substrates (i) a qualitative filter paper and (ii) phenolic paper circuit board material. First, specific patterns were wax printed on A4 size filter paper using a commercial Xerox ColorQube 8570 printer (Xerox, Brazil). The patterns consist of small white rectangles (1.1×1.7 cm) to delimit the electrochemical cell on paper substrates. In a single A4 size paper, 80 patterns were printed, thus affording 80 disposable ePADs. Following, the screen-printing process was performed in the previously patterned paper using electrically conductive carbon and Ag/AgCl inks (Creative Materials, USA) to fabricate the working/auxiliary electrodes and reference electrodes, respectively. The printed filter paper sheets were then placed in a thermal oven for 30 minutes at 100° C. The heating process induces the curing step of the conductive tracks and melts the deposited wax layer that then penetrated in the cellulosic structure, forming a 3D hydrophobic barrier around the hydrophilic patterns (electrochemical cell). Finally, the electrochemical paper-based analytical devices (ePADs) were cut with scissors and the backside of the devices was covered with a transparent tape to prevent solution leakage through the device and to add structural integrity. The phenolic paper is a material largely used as a printed circuit board substrate. The boards were washed thoroughly with deionized water and isopropyl alcohol. The screen-printing process on the paper phenolic resin was performed using the same design and dimension reported for the filter paper platform. The electrochemical circuit board-based devices (eChip) present a rigid substrate and low wettability that dispenses the use of a hydrophobic barrier. After the curing step of printed electrodes, they were cut into small pieces (2×2 cm) and a non-conductive layer was applied to delimit the electrode area.

Modification of the eChips and ePADs. The electrodes were washed with deionized water and cleaned/activated electrochemically by cyclic voltammetry (CV) recorded in sulfuric acid solution (0.1 mol L⁻¹) in the potential range from −1.3 to 1.5 V at the scan rate of 100 mV s⁻¹ for 5 cycles. The eChips were dried at room temperature and 4 μL of GA solution (25% in water) was added on the surface of the working electrode using the drop-casting method. After 1 hour, 44 of ACE2 solution (0.32 μg mL⁻¹) prepared in PBS medium was added on top of the working electrode and left to dry at room temperature for 1.5 hours. Subsequently, 44 of BSA solution (1 mg mL⁻¹) was added on the surface of the working electrode to stabilize the protein and block unspecific sites of the electrode. After 30 minutes, 44 of Nafion solution (1.0% in PBS) was added to the working electrode's surface and left for 1 hour before the final washing with deionized water. The ePADs were modified using the same protocol but applying 2.5-fold higher volume of the modifying agent solutions.

Electrochemical measurements. SquidStat Plus (Admiral Instruments) and Sensit Smart (PalmSens) potentiostats controlled by a laptop running the software SquidStat and a smartphone running the software PSTouch, respectively, were used to record all electrochemical data. The electrodes were characterized by CV technique using a mixture of 5 mmol L⁻¹ potassium ferricyanide/ferrocyanide in the medium of 0.1 mol L⁻¹ KCl solution prior and after electrode modification using a potential range of 0.7 to −0.3 V at the scan rate of 50 mV s⁻¹. Electrochemical impedance spectroscopy (EIS) was used to characterize the biosensor and for SARS-CoV-2 detection. The EIS measurements were performed using 200 μL of a mixture of 5 mmol L⁻¹ ferricyanide/ferrocyanide prepared in 0.1 mol L⁻¹ KCl solution added after the sample incubation on the electrode (104 of OP/NP or saliva samples) and the gentle washing process using PBS solution to remove the unbound SP/SARS-CoV-2. A sinusoidal signal was applying in the frequency range between 10⁵ and 10-1 s⁻¹ using a typical open circuit potential of 0.15 V and an amplitude of 10 mV at room temperature.

Optimization tests. We evaluated the main experimental parameters and processes that affect the efficiency of the developed biosensor. For modification steps, both GA and BSA were used at high concentration levels to ensure the complete recovery of the electrode surface providing the best condition to covalently attachment of ACE2 and its stabilization. The formation of permselective membrane was evaluated by using different Nafion concentrations in the range of 0.5 to 3.0 wt %. After the biosensor preparation, we evaluate its response to different concentrations (1 pg mL⁻¹-10 μg mL⁻¹) of angiotensin II (AngII), the natural substrate of ACE2, to verify if the anchoring and stabilization strategies maintain the biological activity of ACE2. To assess the kinetics of interaction between SP and the architecture of the modified electrode, we carried out calibration curves ranging from 1 pg mL⁻¹ to 1 ng mL⁻¹ SP using different times of incubation (from 1-10 minutes) to obtain the best analytical response to DETECT 1.0. Finally, the need for sample pretreatment of saliva samples was evaluated using 3 different approaches: (i) direct use of raw saliva, (ii) 2 minutes of centrifugation at 10,000 rpm, and (iii) simple dilution of sample 1:1 (v/v) with PBS. We performed this study with saliva samples because it presents greater matrix complexity (high viscosity and content of proteins, lipids, and other biomolecules that can cause biofouling of the electronic surface) when compared to NP/OP swab samples.

Cross-reactivity experiments. Cross-reactivity assays were carried out by exposing the sensor to three coronaviruses (MHV—murine hepatitis virus at 10⁸ PFU mL⁻¹, HCoV-OC43—human coronavirus OC43 at 10⁴ PFU mL⁻¹, and human coronavirus 229E at 10⁷ PFU mL⁻¹), and four non-coronavirus viral strains (H1N1—A/California/2009, H3N2—A/Nicaragua, Influenza B—B/Colorado, HSV2—herpes simplex virus-2, all at 10⁵ PFU mL⁻¹) were used to assess the specificity of our biosensor. The conditions used were the same as those used for all SARS-CoV-2 samples: incubation time of 5 minutes, 10 μL of virus sample, and EIS measurements as specified above (Electrochemical Measurements section).

Quantification and statistical analysis. Cyclic voltammetry and electrochemical impedimetric spectroscopy measurements are presented as an average of 3 or 7 different replicates for each condition and it is described in each figure caption. Graphs were created and statistical tests conducted in GraphPad Prism 8.

### Example 10—Cohort Study

To assess the clinical performance of the instant diagnostic platform, an accuracy study was conducted for detecting SARS-CoV-2 in anterior nare samples and compared the results obtained to those from RT-PCR.

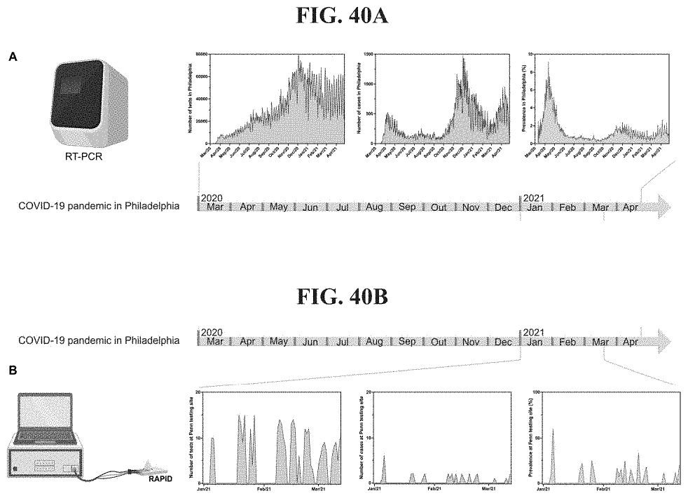

Clinical enrollment was performed over the period of 10 weeks between January and March 2021, following the period with the most COVID-19 cases in Philadelphia (from November to December 2020), where an average of 40,000 tests were performed with around 500 daily COVID-19 cases confirmed (prevalence of ˜1.25% from November to December) (FIG. 40A). All samples collected for the study were aliquoted and frozen at −80° C. promptly after collection. The anterior nare samples were immersed in VTM following the Food and Drug Administration (FDA) recommendation for regulatory applications. A total of 321 nare swab samples were analyzed from incoming patients that agreed to donate their samples.

Clinical samples were incubated for 2 minutes onto the surface of the electrode, as this was the optimal amount of time needed to ensure viral detection using the inventive RAPID system (Torres M D T, et al. (2021) Low-cost biosensor for rapid detection of SARS-CoV-2 at the point-of-care. Matter 4:1-14). The configuration of the modified electrode favors rapid interaction kinetics between the SARS-CoV-2 spike protein and immobilized ACE2 (kinetics constant rate of 10⁴M⁻¹s⁻¹ (Yang J, et al. (2020) Molecular interaction and inhibition of SARS-CoV-2 binding to the ACE2 receptor. Nat Commun 11(1):4541). The RAPID system provides a result within 4 minutes (2 minutes of sample incubation+2 minutes to perform the EIS analysis), which is faster than currently available methods for diagnosing COVID-19 (Bhalla N, et al. (2020) Opportunities and Challenges for Biosensors and Nanoscale Analytical Tools for Pandemics: COVID-19. ACS Nano 14(7):7783-7807). An additional 4 minutes was needed to run each blank, however we did not consider this when calculating our testing time because the blanking step is performed prior to clinical sample analysis. Before starting our clinical study, we calibrated our biosensor using tittered solutions of inactivated SARS-CoV-2 ranging from 10¹ to 10⁶ PFU mL⁻¹. FIG. 40A shows the number of tests, number of cases, and prevalence of COVID-19 in Philadelphia as per official records (COVID data for Pennsylvania (2021) Commonw Pennsylvania). FIG. 40B shows the number of tests, number of cases, and prevalence in the present retrospective cohort study. Complete clinical data paired with the gold-standard method (RT-PCR) were used to confirm the COVID-19 status of each of the 321 samples (FIG. 22B). A total of 31 positive and 290 negative COVID-19 samples were obtained. As provided in Table 8′, below, RAPID demonstrated high sensitivity (80.7%), specificity (89.0%), and accuracy (88.2%).

TABLE 8
Clinical assessment of RAPID detection of SARS-CoV-2 Positive and
negative values obtained by RT-qPCR, and sensitivity, specificity, and accuracy
of RAPID 1.0 using nare samples.

| | RT-qPCR | | | | | | |
|---|---|---|---|---|---|---|---|
| | Positive | Negative | Total | | | | |
| RAPID | (N = 31) | (N = 290) | (N = 321) | Sensitivity | Specificity | Prevalence | Accuracy |
| Positive | 25 | 32 | 57 | 25/31 | | | |
| | | | | (80.6%) | | | |
| Negative | 6 | 258 | 264 | | 258/290 | 31/321 | 283/321 |
| | | | | | (89.0%) | (9.7%) | (88.2%) |

The presence or absence of symptoms and other medical conditions did not interfere with the results obtained with RAPID, and no correlation was found between other medical conditions, race, gender or age with the false positives and negative data obtained. Compared to other electrochemical methods, molecular tests, colorimetric assays, and diagnostic tests reported in the literature, RAPID presents the highest sensitivity reported to date (LOD of 2.8 fg mL⁻¹ SARS-CoV-2 spike protein). In addition, RAPID displays a rapid detection time for SARS-CoV-2 (4 minutes) and is low cost (<US$5.00) (Parihar A, et al. (2020) Point-of-Care Biosensor-Based Diagnosis of COVID-19 Holds Promise to Combat Current and Future Pandemics. ACS Appl Bio Mater 3(11):7326-7343).

Currently available diagnostic tests (prior to the present disclosure) do not provide an accurate, rapid, and affordable diagnosis of COVID-19. For instance, commercial SARS-CoV-2 antigen tests only detect virus concentrations characteristic of later stages of the disease at which patients are already highly infectious (Corman V M, et al. (2021) The Lancet Microbe. doi:10.1016/S2666-5247(21)00056-2), thus not accurately controlling viral spread. RT-PCR, the current gold standard for testing, presents optimal accuracy 3-5 days after the onset of symptoms (Boum Y, et al. (2021). Lancet Infect Dis. doi:10.1016/S1473-3099(21)00132-8). The affordability aspect is also particularly important in order to ensure health equity and increased access to valuable tools, such as diagnostic tests, for preventing viral spread in disadvantaged communities.

In the present cohort study, the performance of RAPID was assessed using 321 anterior nare swab samples from a diversified pool of subjects with age ranging from 18 to 78 years old, different races, genders, COVID-19 related symptoms and other medical conditions (Table 9′, below).

TABLE 9′
Demographic information of the subjects tested.

| | Total | | Positive | | Negative | |
|---|---|---|---|---|---|---|
| | Cohort | | Subjects | | Subjects | |
| | (n = 321) | | (n = 31) | | (n = 290) | |
| Median Age | 37 | (13) | 36 | (14) | 37 | (13) |
| Gender | | | | | | |
| Male | 91 | (28%) | 9 | (29%) | 82 | (28%) |
| Female | 230 | (72%) | 22 | (71%) | 208 | (72%) |
| Race | | | | | | |
| Caucasian | 133 | (41%) | 13 | (42%) | 120 | (41%) |
| African American | 147 | (46%) | 16 | (52%) | 131 | (45%) |
| Hispanic | 13 | (4%) | 2 | (6%) | 11 | (4%) |
| Other | 29 | (9%) | 0 | | 29 | (10%) |
| Medical Problems | | | | | | |
| Asthma | 66 | (21%) | 7 | (23%) | 59 | (20%) |
| Hypertension | 61 | (19%) | 8 | (26%) | 53 | (18%) |
| History of Smoking | 41 | (13%) | 3 | (10%) | 38 | (13%) |
| Diabetes | 28 | (9%) | 5 | (16%) | 23 | (8%) |
| No Medical History | 176 | (55%) | 16 | (52%) | 160 | (55%) |
| Symptoms | | | | | | |
| Cough | 93 | (29%) | 14 | (45%) | 79 | (27%) |
| Headache | 68 | (21%) | 11 | (35%) | 57 | (20%) |
| Fever/Chills | 67 | (21%) | 11 | (35%) | 56 | (19%) |
| Shortness of Breath | 33 | (10%) | 3 | (10%) | 30 | (10%) |
| No Symptoms | 127 | (40%) | 6 | (19%) | 121 | (42%) |

The clinical prevalence of positive COVID-19 cases in the set of samples analyzed was 9.7%, which is higher than the mean observed for the same period in Philadelphia (1-2%; FIG. 40). We did not find statistical correlations between the erroneously diagnosed samples by RAPID and the clinical status or any relevant information obtained from the participants (Table 1′). False-positive results may be due to the use of angiotensin-converting enzyme inhibitors or angiotensin receptor blockers that may interact with RAPID's ACE2-modified electrode. However, the lack of information about the medication usage of participants limited our ability to draw such a correlation. Another important source of potential errors is the self-collection of swabs that took place during testing, as this may lead to samples with no (or very few) viral counts even though the patient was COVID-19 positive and had a medium-to-high viral load.

Additional details concerning the performance of the present cohort study are as follows.

### RAPID Biosensor Preparation.

The testing platform comprised two components: the electrochemical sensor and a potentiostat. The electrochemical sensors were prepared following established protocols (Torres M D T, et al. (2021) Low-cost biosensor for rapid detection of SARS-CoV-2 at the point-of-care. Matter 4:1-14). Briefly, the portable devices were screen-printed in a three-electrode configuration cell on phenolic circuit board material (2×2 cm). Electrically conductive carbon and Ag/AgCl inks were used for the screen-printing process to fabricate the working/auxiliary electrodes and reference electrodes, respectively. The working electrode's carbon surface was modified using the drop-casting method. First, 4 μL of 25% glutaraldehyde (GA) solution was added for 1 hour at 37° C. to allow the formation of a cross-linked polymer, which enabled subsequent anchoring of ACE2 (4 μL at 0.32 mg mL⁻¹). ACE2 was then incubated at 37° C. for 1.5 hours. Next, 4 μL of bovine serum albumin (BSA) were added at 1 mg mL⁻¹ and allowed the working electrode (WE) to dry for 0.5 hours at 37° C. to stabilize the enzyme and block potential active sites present within the carbon electrode, in order to avoid nonspecific adsorption of other proteins to the glutaraldehyde layer and ensure stabilization of the ACE2 tertiary structure. Since the goal was to simplify the detection of SARS-CoV-2 in complex biological samples, such as anterior nare swabs, a 1 wt. % Nafion solution was added as an additional protective layer. Nafion, an anionic and selective membrane that allows the permeation of cationic species, is commonly used to enhance the sensitivity and robustness of electrochemical sensors (Mauritz K A, Moore R B (2004) State of Understanding of Nafion. Chem Rev 104(10):4535-4586). In the present study, the membrane formed by 1 wt. % Nafion solution enhanced the sensitivity of RAPID 1.0, by enabling chemical preconcentration of cation species and protecting the electrode's surface against biofouling by macromolecules present in biological samples, such as proteins and lipids (e Silva R F, et al. (2020) Simple and inexpensive electrochemical paper-based analytical device for sensitive detection of Pseudomonas aeruginosa. Sensors Actuators B Chem 308:127669).

### Anterior Nare Sample Collection and Processing.

The collection of the anterior nare samples was performed by the subjects tested under supervision by clinical research staff at the Penn Presbyterian Medical Center (PPMC). All the demographic information, as well as the presence or absence of symptoms of the individuals tested, are shown in Table 9′, above. The samples were stabilized and stored in viral transport medium (VTM) following CDC guidelines (CDC SOP #: DSR-052-05). The anterior nare samples were maintained on ice during the collection period, separated into identical aliquots and subsequently stored at −80° C. until tested. Care was taken to ensure samples were thawed only once before testing.

### RAPID Test for SARS-CoV-2 Diagnosis.

SquidStat Plus (Admiral Instruments) and MultiAutolab M101 (NOVA 2.1) potentiostats controlled by a laptop running the software SquidStat and a smartphone running the software PSTouch, respectively, were used to record all electrochemical data. The electrodes were characterized by Cyclic Voltammetry (CV) and EIS techniques using a mixture of 5 mmol L⁻¹ potassium ferricyanide/ferrocyanide in 0.1 mol L⁻¹ KCl solution before and after electrode modification with glutaraldehyde, ACE2, BSA, and Nafion. CVs and EIS were recorded using a potential ranging from 0.7 to −0.3 V at the scan rate of 50 mV s⁻¹ and a frequency ranging from 10⁵ to 10⁻¹ Hz using a sinusoidal signal with 10 mV of amplitude at room temperature, respectively.

RAPID reports the selective binding between ACE2, the biological receptor immobilized on the electrode surface, and SARS-CoV-2 spike protein, its binding element. The interaction between these two molecules causes a change in interfacial electron transfer kinetics between the redox probe, ferricyanide/ferrocyanide in solution and the conducting electrode sites. This electrochemical change is then detectable by monitoring the charge-transfer resistance (R_{CT}) and the diameter of the semi-arc on the Nyquist plot, which correlates with the number of spike protein molecules bound to the electrode's surface (5). The selectivity of an EIS biosensor mostly relies on the specificity between the target and the recognizing bioelement immobilized on the electrode surface, and the robustness of the latter to minimize non-specific binding or adsorption of other biomolecules present in biofluids. The EIS measurements were performed using 2004 of a mixture of 5 mmol L⁻¹ ferricyanide/ferrocyanide prepared in a 0.1 mol L⁻¹ KCl solution added after incubating the clinical sample (104 of anterior nare sample) for 2 minutes on electrode surface. A gentle washing step using PBS was performed to remove the sample and any unbound SARS-CoV-2. For the EIS measurement, a sinusoidal signal was applied at room temperature in the frequency range between 10⁵ and 10-1 s⁻¹ using a typical open circuit potential of 0.15 V and an amplitude of 10 mV.

RAPID enables viral detection of SARS-CoV-2 in anterior nare samples stored in VTM within 4 minutes (2 minutes of incubation and 2 minutes of measurement time). Each test was performed at room temperature requiring only a potentiostat, PBS, and a redox probe solution (i.e., mixture of 5 mmol L⁻¹ ferricyanide/ferrocyanide prepared in 0.1 mol L⁻¹ KCl solution). Each RAPID test cost $4.67 to produce ($0.07 to produce the bare electrode, $4.50 to functionalize the electrode with the recognition agent ACE2, and $0.10 to coat the electrode with GA, BSA, and Nafion). RAPID display high sensitivity (1.16 PFU mL⁻¹) comparable to that of RT-PCR assays (1-10 PFU mL⁻¹).

### RT-PCR Analysis.

For the RT-PCR assays, RNA was extracted and purified using the QIAmp DSP Viral RNA Mini Kit (Qiagen) from a 140 μL aliquot. The first step of this process chemically inactivated the virus from the anterior nare samples under highly denaturing conditions (guanidine thiocyanate) and was performed in a biosafety cabinet under BSL-2 enhanced protocols. The remainder of the process was performed at the lab bench under standard conditions using the vacuum protocol as per manufacturer's instructions. Next, RNA present in the samples was analyzed in duplicate using the TaqPath™ 1-Step RT-qPCR reagent (Life Technologies) on the Quantstudio 7 Flex Genetic Analyzer (ABI). The oligonucleotide primers and probes for detection of 2019-nCoV were selected from regions of the virus nucleocapsid (N) gene. The panel was designed for specific detection of the 2019-nCoV viral RNA (two primer/probe sets, N1 and N2). An additional primer/probe set to detect the human RNase P gene (RP) in control samples and clinical specimens was also included in the panel (2019-nCoVEUA-01). RNaseP is a single copy human-specific gene and can indicate the number of human cells collected.

### Prospective Cohort Study Design and Participants.

The performance of RAPID was assessed using both SARS-CoV-2-positive and negative samples from an ambulatory COVID-19 testing site for the general public, led by staff at the Penn Presbyterian Medical Center (PPMC). All participants underwent anterior nare testing for SARS-CoV-2 using CLIA-approved RT-PCR by PPMC staff for testing, and subsequent to this testing underwent study procedures. Adult (age >17 y) subjects were eligible if they (1) underwent PPMC staff-led testing immediately prior to study enrollment, (2) were deemed competent for written consent, (3) were English fluent, and (4) did not have any contraindications to anterior nare samples collection procedures, such as recent facial surgery or active head and neck cancer. Subjects completed standard written consent, and then completed a short survey including demographic information and recent infectious symptoms, if any. Subjects then underwent anterior nasal swabbing supervised by trained clinical research coordinators. This work was approved by the University of Pennsylvania Institutional Review Board (IRB 844145).

### Diagnosis and Statistical Analysis.

The R_{CT} values of Nyquist plots obtained using Squidstat Plus (Admiral Instruments) and Multi Autolab M101 (Metrohm) were extracted by the application of an equivalent circuit using the softwares Zahner Analysis and Nova 2.1, respectively. The equivalent circuit comprises two semi-arc regions observed in the Nyquist plots, where the first is a non-defined semi-arc at a high-frequency range due to inhomogeneity or defects in the electrode modification step (during drop-casting functionalization) and considerably small (R_{CT}˜10Ω) (Uygun Z O, Ertu\check{g}rul Uygun HD (2014) A short footnote: Circuit design for faradaic impedimetric sensors and biosensors. Sensors Actuators B Chem 202:448-453; Bertok T, et al. (2019) Electrochemical Impedance Spectroscopy Based Biosensors: Mechanistic Principles, Analytical Examples and Challenges towards Commercialization for Assays of Protein Cancer Biomarkers. ChemElectroChem 6(4):989-1003). The second parallel component of the equivalent circuit comprises an R_{CT}, whose signal intensity was proportional to the logarithm of the concentration of SARS-CoV-2 and presented a Warburg element to describe the mass transport (diffusional control).

To diagnose a given sample, the normalized R_{CT}, defined by the following equation, was used:

$$normalized {R}_{CT} = \frac{Z - {Z}_{0}}{{Z}_{0}}$$

where Z is the R_{CT} of the sample and Z₀ is the R_{CT} of the blank solution (VTM).

A cut-off value was set as a 10% change in the R_{CT} when compared to the blank solution. Such a cut-off threshold considers the LOQ value previously obtained for inactivated virus, thus allowing discrimination between SARS-CoV-2 negative and SARS-CoV-2 positive samples.

The presently disclosed RAPID system is an inexpensive and portable alternative to existing COVID-19 tests, allowing for decentralized diagnosis at the point-of-care. The fast detection (4 min) enabled by the present approach is significantly lower than commercially available tests, and could potentially be lowered even more by using alternative recognition agents, such as engineered versions of human ACE2 with enhanced selective binding towards SARS-CoV-2, or engineered receptors to the SARS-CoV-2 spike protein, such as antibodies (Chan K K, et al. (2020). Science (80-) 369(6508):1261-1265).

Finally, RAPID can be multiplexed to allow detection of emerging biological threats such as bacteria, fungi, and other viruses, simply by adding other recognition agents and modifying the electrodes disposition (array configuration). Its ability to detect minimal viral particles within a sample allows diagnosing COVID-19 at the onset of the infection. Collectively, its low-cost, rapid detection time, and high analytical sensitivity make RAPID an exciting alternative tool for high-frequency COVID-19 testing and effective population surveillance (Mina M J, et al. (2020) N Engl J Med 383(22):e120).

### REFERENCES I

The following publications may be relevant to the presently disclosed subject matter relating to SARS-CoV-2 biosensors described under Section I above:
1. Coronavirus disease (COVID-19), World Health Organization. https://www.who.int/emergencies/diseases/novel-coronavirus-2019 (Apr. 11, 2022).
2. B. D. Kevadiya, et al., Diagnostics for SARS-CoV-2 infections. Nat. Mater. 20, 593-605 (2021).
3. A. L. Ferreira, L. F. de Lima, M. D. T. Torres, W. R. de Araujo, C. de la Fuente-Nunez, Low-Cost Optodiagnostic for Minute-Time Scale Detection of SARS-CoV-2. ACS Nano 15, 17453-17462 (2021).
4. L. F. de Lima, A. L. Ferreira, M. D. T. Torres, W. R. de Araujo, C. de la Fuente-Nunez, Minute-scale detection of SARS-CoV-2 using a low-cost biosensor composed of pencil graphite electrodes. Proc. Natl. Acad. Sci. 118, e2106724118 (2021).
5. V. M. Moreira, et al., Diagnosis of SARS-cov-2 infection by RT-PCR using specimens other than naso- And oropharyngeal swabs: A systematic review and meta-analysis. Diagnostics 11 (2021).
6. G. Seo, et al., Rapid Detection of COVID-19 Causative Virus (SARS-CoV-2) in Human Nasopharyngeal Swab Specimens Using Field-Effect Transistor-Based Biosensor. ACS Nano 14, 5135-5142 (2020).
7 M. Letko, A. Marzi, V. Munster, Functional assessment of cell entry and receptor usage for SARS-CoV-2 and other lineage B betacoronaviruses. Nat. Microbiol. 5, 562-569 (2020).
8. W. T. Harvey, et al., SARS-CoV-2 variants, spike mutations and immune escape. Nat. Rev. Microbiol. 19, 409-424 (2021).
9. N. Zhang, et al., Recent advances in the detection of respiratory virus infection in humans. J. Med. Virol. 92, 408-417 (2020).
10. Z. Zhao, et al., Advancements in electrochemical biosensing for respiratory virus detection: A review. TrAC—Trends Anal. Chem. 139, 116253 (2021).
11. T. A. Baldo, et al., Wearable and Biodegradable Sensors for Clinical and Environmental Applications. ACS Appl. Electron. Mater. 3, 68-100 (2021).
12. J. Guo, J. Li, Q. Rao, Z. Xu, Phenolic Molding Compound Filled with Nonmetals of Waste PCBs. Environ. Sci. Technol. 42, 624-628 (2008).
13. J. M. Millican, S. Agarwal, Plastic Pollution: A Material Problem? Macromolecules 54, 4455-4469 (2021).
14. S. Swingler, et al., Recent advances and applications of bacterial cellulose in biomedicine. Polymers (Basel). 13, 1-29 (2021).
15. N. O. Gomes, E. Carrilho, S. A. S. Machado, L. F. Sgobbi, Bacterial cellulose-based electrochemical sensing platform: A smart material for miniaturized biosensors. Electrochim. Acta 349, 136341 (2020).
16. S. B. Schröpfer, et al., Biodegradation evaluation of bacterial cellulose, vegetable cellulose and poly (3-hydroxybutyrate) in soil. Polimeros 25, 154-160 (2015).
17. J. Kucińska-Lipka, I. Gubanska, H. Janik, Bacterial cellulose in the field of wound healing and regenerative medicine of skin: recent trends and future prospectives. Polym. Bull. 72, 2399-2419 (2015).
18. F. G. Torres, O. P. Troncoso, K. N. Gonzales, R. M. Sari, S. Gea, Bacterial cellulose-based biosensors. Med. DEVICES SENSORS 3, e10102 (2020).
19. M. Florea, et al., Engineering control of bacterial cellulose production using a genetic toolkit and a new cellulose-producing strain. Proc. Natl. Acad. Sci. 113 (2016).
20. M. J. Mina, R. Parker, D. B. Larremore, Rethinking Covid-19 Test Sensitivity—A Strategy for Containment. N Engl. J. Med. 383, e120 (2020).
21. J. Ding, W. Qin, Trends in Analytical Chemistry Recent advances in potentiometric biosensors. Trends Anal. Chem. 124, 115803 (2020).
22. H. Karimi-maleh, Y. Orooji, F. Karimi, M. Alizadeh, A critical review on the use of potentiometric based biosensors for biomarkers detection. Biosens. Bioelectron. 184, 113252 (2021).
23. S. M. Abdelbasir, et al., Graphene-Anchored Cuprous Oxide Nanoparticles from Waste Electric Cables for Electrochemical Sensing. ACS Sustain. Chem. Eng. 6, 12176-12186 (2018).
24. M. J. MacLeod, J. A. Johnson, PEGylated N-Heterocyclic Carbene Anchors Designed to Stabilize Gold Nanoparticles in Biologically Relevant Media. J. Am. Chem. Soc. 137, 7974-7977 (2015).
25. A. L. Ferreira, et al., Development of a novel biosensor for Creatine Kinase (CK-MB) using Surface Plasmon Resonance (SPR). Appl. Surf. Sci., 149565 (2021).
26. R. Balint, N. J. Cassidy, S. H. Cartmell, Conductive polymers: Towards a smart biomaterial for tissue engineering. Acta Biomater. 10, 2341-2353 (2014).
27. M. D. T. Tones, W. R. de Araujo, L. F. de Lima, A. L. Ferreira, C. de la Fuente-Nunez, Low-cost biosensor for rapid detection of SARS-CoV-2 at the point of care. Matter 4, 2403-2416 (2021).
28. B. A. E. Lehner, et al., Biocompatible Graphene Oxide Nanosheets Densely Functionalized with Biologically Active Molecules for Biosensing Applications. ACS Appl. Nano Mater. 4, 8334-8342 (2021).
29. L. Hlavatá, V. Vyskočil, K. Beniková, M. Borbélyová, J. Labuda, DNA-based biosensors with external Nafion and chitosan membranes for the evaluation of the antioxidant activity of beer, coffee, and tea. Cent. Eur. J. Chem. 12, 604-611 (2014).
30. R. F. e Silva, T. R. Longo Cesar Paixão, M. Der Torossian Torres, W. R. de Araujo, Simple and inexpensive electrochemical paper-based analytical device for sensitive detection of Pseudomonas aeruginosa. Sensors Actuators, B Chem. 308, 127669 (2020).
31. M. D. T. Torres, et al., Detection of SARS-CoV-2 with RAPID: A prospective cohort study. iScience 25, 104055 (2022).
32. M. Nasrollahzadeh, M. Sajjadi, G. J. Soufi, S. Iravani, R. S. Varma, Nanomaterials and nanotechnology-associated innovations against viral infections with a focus on coronaviruses. Nanomaterials 10, 1072 (2020).
33. J. W. A. Findlay, R. F. Dillard, Appropriate calibration curve fitting in ligand binding assays. AAPS J. 9, E260—E267 (2007).
34. P. G. Gottschalk, J. R. Dunn, The five-parameter logistic: A characterization and comparison with the four-parameter logistic. Anal. Biochem. 343, 54-65 (2005).
35. Y. Fong, J. Wakefield, S. De Rosa, N. Frahm, A Robust Bayesian Random Effects Model for Nonlinear Calibration Problems. Biometrics 68, 1103-1112 (2012).
36. C. A. Holstein, M. Griffin, J. Hong, P. D. Sampson, Statistical Method for Determining and Comparing Limits of Detection of Bioassays. Anal. Chem. 87, 9795-9801 (2015).
37. A. Yakoh, et al., Paper-based electrochemical biosensor for diagnosing COVID-19: Detection of SARS-CoV-2 antibodies and antigen. Biosens. Bioelectron. 176, 112912 (2021).
38. S. Eissa, M. Zourob, Development of a Low-Cost Cotton-Tipped Electrochemical Immunosensor for the Detection of SARS-CoV-2. Anal. Chem. 93, 1826-1833 (2021)
39. M. Alafeef, K. Dighe, P. Moitra, D. Pan, Rapid, Ultrasensitive, and Quantitative Detection of SARS-CoV-2 Using Antisense Oligonucleotides Directed Electrochemical Biosensor Chip. ACS Nano 14, 17028-17045 (2020).
40. R. M. Torrente-Rodríguez, et al., SARS-CoV-2 RapidPlex: A Graphene-Based Multiplexed Telemedicine Platform for Rapid and Low-Cost COVID-19 Diagnosis and Monitoring. Matter 3, 1981-1998 (2020).
41. J. Lerdsri, W. Chananchana, J. Upan, T. Sridara, J. Jakmunee, Label-free colorimetric aptasensor for rapid detection of aflatoxin B1 by utilizing cationic perylene probe and localized surface plasmon resonance of gold nanoparticles. Sensors Actuators, B Chem. 320, 128356 (2020).
42. J. P. Broughton, et al., CRISPR—Cas12-based detection of SARS-CoV-2. Nat. Biotechnol. 38, 870-874 (2020).
43. P. Moitra, et al., Selective Naked-Eye Detection of SARS-CoV-2 Mediated by N Gene Targeted Antisense Oligonucleotide Capped Plasmonic Nanoparticles. ACS Nano 14, 7617-7627 (2020).
44. S. S. Goher, F. Ali, M. Amin, The Delta variant mutations in the receptor binding domain of SARS-CoV-2 show enhanced electrostatic interactions with the ACE2. Med. Drug Discov. 13, 100114 (2022).
45. H. Liu, P. Wei, J. W. Kappler, P. Marrack, G. Zhang, SARS-CoV-2 Variants of Concern and Variants of Interest Receptor Binding Domain Mutations and Virus Infectivity. Front. Immunol. 13, 825256 (2022).
46. F. Konings, et al., SARS-CoV-2 Variants of Interest and Concern naming scheme conducive for global discourse. Nat. Microbiol. 6, 821-823 (2021).
47. S. P. Otto, et al., The origins and potential future of SARS-CoV-2 variants of concern in the evolving COVID-19 pandemic. Curr. Biol. 31, R918—R929 (2021).
48. E. Callaway, Are COVID surges becoming more predictable? New Omicron variants offer a hint. Nature 605, 204-206 (2022).
49. A. F. S. Costa, F. C. G. Almeida, G. M. Vinhas, L. A. Sarubbo, Production of bacterial cellulose by Gluconacetobacter hansenii using corn steep liquor as nutrient sources. Front. Microbiol. 8, 1-12 (2017).

### REFERENCES II

The following publications may be relevant to the presently disclosed subject matter relating to HSV biosensors described under Section II above:
1. James, C., Harfouche, M., Welton, N. J., Turner, M. E., and Abu-raddad, L. J. (2020). Herpes simplex virus: global infection prevalence and incidence estimates, 2016. Bull World Heal. Organ. 98(5), 315-329. 10.2471/BLT.19.237149.
2. Weidmann, M., Meyer-König, U., and Hufert, F. T. (2003). Rapid detection of herpes simplex virus and varicella-zoster virus infections by real-time PCR. J. Clin. Microbiol. 41, 1565-1568. 10.1128/JCM.41.4.1565-1568.2003.
3. Herold, B. C., Visalli, R. J., Susmarski, N., Brandt, C. R., and Spear, P. G. (1994). Glycoprotein C-independent binding of herpes simplex virus to cells requires cell surface heparan sulphate and glycoprotein B. J. Gen. Virol. 75, 1211-1222. 10.1099/0022-1317-75-6-1211.
4. WuDunn, D., and Spear, P. G. (1989). Initial interaction of herpes simplex virus with cells is binding to heparan sulfate. J. Virol. 63, 52-58. 10.1128/jvi.63.1.52-58.1989.
5. Nicola, A. V, Peng, C., Lou, H., Cohen, G. H., and Eisenberg, R. J. (1997). Antigenic structure of soluble herpes simplex virus (HSV) glycoprotein D correlates with inhibition of HSV infection. J. Virol. 71, 2940-2946. 10.1128/jvi.71.4.2940-2946.1997.
6. Eisenberg, R. J., Atanasiu, D., Cairns, T. M., Gallagher, J. R., Krummenacher, C., and Cohen, G. H. (2012). Herpes virus fusion and entry: A story with many characters. Viruses 4, 800-832. 10.3390/v4050800.
7. Johnson, R. M., and Spear, P. G. (1989). Herpes simplex virus glycoprotein D mediates interference with herpes simplex virus infection. J. Virol. 63, 819-827. 10.1128/jvi.63.2.819-827.1989.
8. Johnson, D. C., Burke, R. L., and Gregory, T. (1990). Soluble forms of herpes simplex virus glycoprotein D bind to a limited number of cell surface receptors and inhibit virus entry into cells. J. Virol. 64, 2569-2576. 10.1128/JVI.64.6.2569-2576.1990.
9. Fuller, A. O., and Lee, W. C. (1992). Herpes simplex virus type 1 entry through a cascade of virus-cell interactions requires different roles of gD and gH in penetration. J. Virol. 66, 5002-5012. 10.1128/jvi.66.8.5002-5012.1992.
10. Singh, A., Preiksaitis, J., and Romanowski, B. (2005). The laboratory diagnosis of herpes simplex virus infections. Can. J. Infect. Dis. Med. Microbiol. 16, 92-98. 10.1155/2005/318294.
11. Anderson, N. W., Buchan, B. W., and Ledeboer, N. A. (2014). Light Microscopy, Culture, Molecular, and Serologic Methods for Detection of Herpes Simplex Virus. J. Clin. Microbiol. 52, 2-8. 10.1128/JCM.01966-13.
12. Wald, A., and Ashley-Morrow, R. (2002). Serological testing for herpes simplex virus (HSV)-1 and HSV-2 infection. Clin. Infect. Dis. 35. 10.1086/342104.
13. Johnson, G., Nelson, S., Petric, M., and Tellier, R. (2000). Comprehensive PCR-based assay for detection and species identification of human herpesviruses. J. Clin. Microbiol. 38, 3274-3279. 10.1128/jcm.38.9.3274-3279.2000.
14. Gardella, C., Huang, M. L., Wald, A., Magaret, A., Selke, S., Morrow, R., and Corey, L. (2010). Rapid polymerase chain reaction assay to detect herpes simplex virus in the genital tract of women in labor. Obstet. Gynecol. 115, 1209-1216. 10.1097/AOG.0b013e3181e01415.
15. Narang, J., Singhal, C., Mathur, A., Sharma, S., Singla, V., and Pundir, C. S. (2018). Portable bioactive paper based genosensor incorporated with Zn—Ag nanoblooms for herpes detection at the point-of-care. Int. J. Biol. Macromol. 107, 2559-2565. 10.1016/j.ijbiomac.2017.10.146.
16. Tam, P. D., Tuan, M. A., Aarnink, T., and Chien, N. D. (2008). Directly immobilized DNA sensor for label-free detection of herpes virus. 5th Int. Conf. Inf. Technol. Appl. Biomed. ITAB 2008 conjunction with 2nd Int. Symp. Summer Sch. Biomed. Heal. Eng. IS3BHE 2008, 214-218. 10.1109/ITAB.2008.4570538.
17. Kara, P., Meric, B., Zeytinoglu, A., and Ozsoz, M. (2004). Electrochemical DNA biosensor for the detection and discrimination of herpes simplex Type I and Type II viruses from PCR amplified real samples. Anal. Chim. Acta 518, 69-76. 10.1016/j.aca.2004.04.004.
18. Klunder, K. J., Nilsson, Z., Sambur, J. B., and Henry, C. S. (2017). Patternable Solvent-Processed Thermoplastic Graphite Electrodes. J. Am. Chem. Soc. 139, 12623-12631. 10.1021/jacs.7b06173.
19. Giovine, P., Settembre, E. C., Bhargava, A. K., Luftig, M. A., Lou, H., Cohen, G. H., Eisenberg, R. J., Krummenacher, C., and Carfi, A. (2011). Structure of herpes simplex virus glycoprotein d bound to the human receptor nectin-1. PLoS Pathog. 7. 10.1371/journal.ppat.1002277.
20. Torres, M. D. T., de Araujo, W. R., de Lima, L. F., Ferreira, A. L., and de la Fuente-Nunez, C. (2021). Low-cost biosensor for rapid detection of SARS-CoV-2 at the point of care. Matter 4, 2403-2416. 10.1016/j.matt.2021.05.003.
21. Randles, J. E. B. (1947). Kinetics of rapid electrode reactions. Faraday Discuss. 1, 11-19. 10.1039/DF9470100011.
22. de Lima, L. F., Ferreira, A. L., Tones, M. D. T., de Araujo, W. R., and de la Fuente-Nunez, C. (2021). Minute-scale detection of SARS-CoV-2 using a low-cost biosensor composed of pencil graphite electrodes. Proc. Natl. Acad. Sci. 118, e2106724118. 10.1073/pnas.2106724118.
23. Ferreira, A. L., Lima, L. F. De, Moraes, A. S., Rubira, R. J. G., Constantino, C. J. L., Leite, F. L., and Delgado-silva, A. O. (2021). Development of a novel biosensor for Creatine Kinase (CK-MB) using Surface Plasmon Resonance (SPR). Appl. Surf. Sci., 149565. 10.1016/j.apsusc.2021.149565.
24. Balint, R., Cassidy, N. J., and Cartmell, S. H. (2014). Conductive polymers: Towards a smart biomaterial for tissue engineering. Acta Biomater. 10, 2341-2353. 10.1016/j.actbio.2014.02.015.
25. Li, J., Bai, J., Dong, L., Yang, M., Hu, Y., Gao, L., and Qian, H. (2021). A Novel Electrochemical Biosensor based on Layered Hydroxide Nanosheets/DNA Composite for the Determination of Phenformin Hydrochloride. Int. J. Electrochem. Sci. 16, 1-13. 10.20964/2021.02.05.
26. Farooq, U., Ullah, M. W., Yang, Q., Aziz, A., Xu, J., Zhou, L., and Wang, S. (2020).
High-density phage particles immobilization in surface-modified bacterial cellulose for ultra-sensitive and selective electrochemical detection of Staphylococcus aureus. Biosens. Bioelectron. 157, 112163. 10.1016/j.bios.2020.112163.
27. Deng, C., Chen, J., Nie, Z., and Si, S. (2010). A sensitive and stable biosensor based on the direct electrochemistry of glucose oxidase assembled layer-by-layer at the multiwall carbon nanotube-modified electrode. Biosens. Bioelectron. 26, 213-219. 10.1016/j.bios.2010.06.013.
28. Cruz, J., Kawasaki, M., and Gorski, W. (2000). Electrode coatings based on chitosan scaffolds. Anal. Chem. 72, 680-686. 10.1021/ac990954b.
29. Hanssen, B. L., Siraj, S., and Wong, D. K. Y. (2016). Recent strategies to minimise fouling in electrochemical detection systems. Rev. Anal. Chem. 35, 1-28. 10.1515/revac-2015-0008.
30. Annu, and Raja, A. N. (2020). Recent development in chitosan-based electrochemical sensors and its sensing application. Int. J. Biol. Macromol. 164, 4231-4244. 10.1016/j.ijbiomac.2020.09.012.
31. Suginta, W., Khunkaewla, P., and Schulte, A. (2013). Electrochemical Biosensor Applications of Polysaccharides Chitin and Chitosan. Chem. Rev. 113, 5458-5479. 10.1021/cr300325r.
32. Hlavatá, L., Vyskočil, V., Beniková, K., Borbélyová, M., and Labuda, J. (2014). DNA-based biosensors with external Nafion and chitosan membranes for the evaluation of the antioxidant activity of beer, coffee, and tea. Cent. Eur. J. Chem. 12, 604-611. 10.2478/s11532-014-0516-4.
33. Yang, L., Ren, X., Tang, F., and Zhang, L. (2009). A practical glucose biosensor based on Fe3O4 nanoparticles and chitosan/nafion composite film. Biosens. Bioelectron. 25, 889-895. 10.1016/j.bios.2009.09.002.
34. Kang, H., Jung, S., Jeong, S., Kim, G., and Lee, K. (2015). Polymer-metal hybrid transparent electrodes for flexible electronics. Nat. Commun. 6, 1-7. 10.1038/ncomms7503.
35. Farmani, M. R., Peyman, H., and Roshanfekr, H. (2020). Blue luminescent graphene quantum dot conjugated cysteamine functionalized-gold nanoparticles (GQD-AuNPs) for sensing hazardous dye Erythrosine B. Spectrochim. Acta—Part A Mol. Biomol. Spectrosc. 229, 117960. 10.1016/j.saa.2019.117960.
36. Gottschalk, P. G., and Dunn, J. R. (2005). The five-parameter logistic: A characterization and comparison with the four-parameter logistic. Anal. Biochem. 343, 54-65. 10.1016/j.ab.2005.04.035.
37. Holstein, C. A., Griffin, M., Hong, J., and Sampson, P. D. (2015). Statistical Method for Determining and Comparing Limits of Detection of Bioassays. Anal. Chem. 87, 9795-9801. 10.1021/acs.analchem.5b02082.
38. Tam, P. D., Tuan, M. A., Huy, T. Q., Le, A. T., and Hieu, N. Van (2010). Facile preparation of a DNA sensor for rapid herpes virus detection. Mater. Sci. Eng. C 30, 1145-1150. 10.1016/j.msec.2010.06.010.
39. Toldrá, A., Furones, M. D., O'Sullivan, C. K., and Campàs, M. (2020). Detection of isothermally amplified ostreid herpesvirus 1 DNA in Pacific oyster (Crassostrea gigas) using a miniaturised electrochemical biosensor. Talanta 207, 120308. 10.1016/j.talanta.2019.120308.
40. Garcia, L. F., Silvio Batista Rodrigues, E., Rocha Lino de Souza, G., Jubé Wastowski, I., Mota de Oliveira, F., Tones Pio dos Santos, W., and Souza Gil, E. (2020). Impedimetric Biosensor for Bovine Herpesvirus Type 1-Antigen Detection. Electroanalysis 32, 1100-1106. 10.1002/elan.201900606.
41. Loughman, T., Singh, B., Seddon, B., Noone, P., and Santhosh, P. (2017). Validation of a membrane touch biosensor for the qualitative detection of IgG class antibodies to herpes simplex virus type 2. Analyst 142, 2725-2734. 10.1039/C7AN00666G.
42. Nahar, S., Ahmed, M. U., Safavieh, M., Rochette, A., Toro, C., and Zourob, M. (2015). A flexible and low-cost polypropylene pouch for naked-eye detection of herpes simplex viruses. Analyst 140, 931-937. 10.1039/c4an01701c.
43. Kessler, H. H., Mühlbauer, G., Rinner, B., Stelzl, E., Berger, A., Dörr, H. W., Santner, B., Marth, E., and Rabenau, H. (2000). Detection of herpes simplex virus DNA by real-time PCR. J. Clin. Microbiol. 38, 2638-2642. 10.1128/jcm.38.7.2638-2642.2000.
44. Perkins, J. D. (2007). Detection and Diagnosis of Herpes Simplex Virus Infection in Adults with Acute Liver Failure. Liver Transplant. 13, 767-768. 10.1002/1t.
45. Burrows, J., Nitsche, A., Bayly, B., Walker, E., Higgins, G., and Kok, T. (2002). Detection and subtyping of Herpes simplex virus in clinical samples by LightCycler PCR, enzyme immunoassay and cell culture. BMC Microbiol. 2, 1-7. 10.1186/1471-2180-2-12.
46. Dominguez, S. R., Pretty, K., Hengartner, R., and Robinson, C. C. (2018). Comparison of herpes simplex virus PCR with culture for virus detection in multisource surface swab specimens from neonates. J. Clin. Microbiol. 56, 1-5. 10.1128/JCM.00632-18.
47. Krumbholz, A., Schafer, M., Lorentz, T., and Sauerbrei, A. (2019). Quadruplex real-time PCR for rapid detection of human alphaherpesviruses. Med. Microbiol. Immunol. 208, 197-204. 10.1007/s00430-019-00580-2.
48. MultiCode®-RTx HSV 1&2 Kit For In Vitro Diagnostic Use Real-Time PCR Qualitative Detection and Typing of HSV-1 or HSV-2. https://www.ld.ru/w/multiplex/HSV-Kit-Brochure.pdf
49. BD ProbeTec™ Herpes Simplex Viruses (HSV 1 & 2) Q×Amplified DNA Assays Sensitivity Advances Clinical Performance. https://handicare.blob.core.windows.net/media/254942/brosjyre-hsv.pdf
50. Kim, H. J., Tong, Y., Tang, W., Quimson, L., Cope, V. A., Pan, X., Motre, A., Kong, R., Hong, J., Kohn, D., et al. (2011). A rapid and simple isothermal nucleic acid amplification test for detection of herpes simplex virus types 1 and 2. J. Clin. Virol. 50, 26-30. 10.1016/J.JCV.2010.09.006.
51. Geraghty, R. J., Fridberg, A., Krummenacher, C., Cohen, G. H., Eisenberg, R. J., and Spear, P. G. (2001). Use of chimeric nectin-1(HveC)-related receptors to demonstrate that ability to bind alphaherpesvirus gD is not necessarily sufficient for viral entry. Virology 285, 366-375. 10.1006/viro.2001.0989.
52. Ferreira, A. L., de Lima, L. F., Torres, M. D. T., de Araujo, W. R., and de la Fuente-Nunez, C. (2021). Low-Cost Optodiagnostic for Minute-Time Scale Detection of SARS-CoV-2. ACS Nano 15, 17453-17462. 10.1021/acsnano.1c03236.
53. Elveborg, S., Monteil, V., and Mirazimi, A. (2022). Methods of Inactivation of Highly Pathogenic Viruses for Molecular, Serology or Vaccine Development Purposes. Pathogens 11, 271. 10.3390/pathogens11020271.
54. Carfi, A., Willis, S. H., Whitbeck, J. C., Krummenacher, C., Cohen, G. H., Eisenberg, R. J., and Wiley, D. C. (2001). Herpes Simplex Virus Glycoprotein D Bound to the Human Receptor HveA. Mol. Cell 8, 169-179. 10.1016/S1097-2765(01)00298-2.
55. Krummenacher, C., Nicola, A. V., Whitbeck, J. C., Lou, H., Hou, W., Lambris, J. D., Geraghty, R. J., Spear, P. G., Cohen, G. H., and Eisenberg, R. J. (1998). Herpes Simplex Virus Glycoprotein D Can Bind to Poliovirus Receptor-Related Protein 1 or Herpesvirus Entry Mediator, Two Structurally Unrelated Mediators of Virus Entry. J. Virol. 72, 7064-7074. 10.1128/JVI.72.9.7064-7074.1998.
56. Hook, L. M., Friedman, H. M., and Awasthi, S. (2021). Guinea Pig and Mouse Models for Genital Herpes Infection. Curr. Protoc. 1, 32. 10.1002/cpz1.332.
57. Awasthi, S., Hook, L. M., Shaw, C. E., Pahar, B., Stagray, J. A., Liu, D., Veazey, R. S., and Friedman, H. M. (2017). An HSV-2 Trivalent Vaccine Is Immunogenic in Rhesus Macaques and Highly Efficacious in Guinea Pigs. PLOS Pathog. 13, e1006141. 10.1371/journal.ppat.1006141.

The following publications may be relevant to the presently disclosed subject matter relating to SARS-CoV-2 biosensors described under Section III above:
Administration, U. S. F.& D. (2020). In Vitro Diagnostics EUAs.
Alafeef, M., Dighe, K., Moitra, P., and Pan, D. (2020). Rapid, Ultrasensitive, and Quantitative Detection of SARS-CoV-2 Using Antisense Oligonucleotides Directed Electrochemical Biosensor Chip. ACS Nano 14, 17028-17045.
Andersen, K. G., Rambaut, A., Lipkin, W. I., Holmes, E. C., and Garry, R. F. (2020). The proximal origin of SARS-CoV-2. Nat. Med. 26, 450-452.
Ataide, V. N., Mendes, L. F., Gama, L. I. L. M., de Araujo, W. R., and Paixão, T. R. L. C. (2020). Electrochemical paper-based analytical devices: ten years of development. Anal. Methods 12, 1030-1054.
Baek, Y. H., Um, J., Antigua, K. J. C., Park, J.-H., Kim, Y., Oh, S., Kim, Y., Choi, W.-S., Kim, S. G., Jeong, J. H., et al. (2020). Development of a reverse transcription-loop-mediated isothermal amplification as a rapid early-detection method for novel SARS-CoV-2. Emerg. Microbes Infect. 9, 998-1007.
Bahadir, E. B., and Sezgintúrk, M. K. (2016). A review on impedimetric biosensors. Artif. Cells, Nanomedicine, Biotechnol. 44, 248-262.
Barbosa, O., Ortiz, C., Berenguer-Murcia, Á., Tones, R., Rodrigues, R. C., and Fernandez-Lafuente, R. (2014). Glutaraldehyde in bio-catalysts design: a useful crosslinker and a versatile tool in enzyme immobilization. RSC Adv. 4, 1583-1600.
Bertok, T., Lorencova, L., Chocholova, E., Jane, E., Vikartovska, A., Kasak, P., and Tkac, J. (2019). Electrochemical Impedance Spectroscopy Based Biosensors: Mechanistic Principles, Analytical Examples and Challenges towards Commercialization for Assays of Protein Cancer Biomarkers. ChemElectroChem 6, 989-1003.
Broughton, J. P., Deng, X., Yu, G., Fasching, C. L., Servellita, V., Singh, J., Miao, X., Streithorst, J. A., Granados, A., Sotomayor-Gonzalez, A., et al. (2020). CRISPR—Cas12-based detection of SARS-CoV-2. Nat. Biotechnol. 38, 870-874.
Chan, K. K., Dorosky, D., Sharma, P., Abbasi, S. A., Dye, J. M., Kranz, D. M., Herbert, A. S., and Procko, E. (2020). Engineering human ACE2 to optimize binding to the spike protein of SARS coronavirus 2. Science (80-.). 369, 1261-1265.
e Silva, R. F., Longo Cesar Paixão, T. R., Der Torossian Torres, M., and de Araujo, W. R. (2020). Simple and inexpensive electrochemical paper-based analytical device for sensitive detection of Pseudomonas aeruginosa. Sensors Actuators B Chem. 308, 127669.
Glasgow, A., Glasgow, J., Limonta, D., Solomon, P., Lui, I., Zhang, Y., Nix, M. A., Rettko, N. J., Zha, S., Yamin, R., et al. (2020). Engineered ACE2 receptor traps potently neutralize SARS-CoV-2. Proc. Natl. Acad. Sci. 117, 28046-28055.
Government, A. C. (2020). Information of Coronavirus (COVID-19) Testing.
Jamal, A. J., Mozafarihashjin, M., Coomes, E., Powis, J., Li, A. X., Paterson, A., Anceva-Sami, S., Barati, S., Crowl, G., Faheem, A., et al. (2020). Sensitivity of Nasopharyngeal Swabs and Saliva for the Detection of Severe Acute Respiratory Syndrome Coronavirus 2. Clin. Infect. Dis. ciaa848.
Jiao, J., Duan, C., Xue, L., Liu, Y., Sun, W., and Xiang, Y. (2020). DNA nanoscaffold-based SARS-CoV-2 detection for COVID-19 diagnosis. Biosens. Bioelectron. 167, 112479.
Kaushik, A. K., Dhau, J. S., Gohel, H., Mishra, Y. K., Kateb, B., Kim, N.-Y., and Goswami, D. Y. (2020). Electrochemical SARS-CoV-2 Sensing at Point-of-Care and Artificial Intelligence for Intelligent COVID-19 Management. ACS Appl. Bio Mater. acsabm.0c01004.
Mauritz, K. A., and Moore, R. B. (2004). State of Understanding of Nafion. Chem. Rev. 104, 4535-4586.
Moitra, P., Alafeef, M., Dighe, K., Frieman, M. B., and Pan, D. (2020). Selective Naked-Eye Detection of SARS-CoV-2 Mediated by N Gene Targeted Antisense Oligonucleotide Capped Plasmonic Nanoparticles. ACS Nano 14, 7617-7627.
Muñoz, J., Montes, R., and Baeza, M. (2017). Trends in electrochemical impedance spectroscopy involving nanocomposite transducers: Characterization, architecture surface and bio-sensing. TrAC Trends Anal. Chem. 97, 201-215.
Ozer, T., McMahon, C., and Henry, C. S. (2020). Advances in Paper-Based Analytical Devices. Annu. Rev. Anal. Chem. 13, 85-109.
Pereira, A. R., Sedenho, G. C., Souza, J. C. P. de, and Crespilho, F. N. (2018). Advances in enzyme bioelectrochemistry. An. Acad. Bras. Cienc. 90, 825-857.
Qiu, G., Gai, Z., Tao, Y., Schmitt, J., Kullak-Ublick, G. A., and Wang, J. (2020). Dual-Functional Plasmonic Photothermal Biosensors for Highly Accurate Severe Acute Respiratory Syndrome Coronavirus 2 Detection. ACS Nano 14, 5268-5277.
Rao, S. N., Manissero, D., Steele, V. R., and Pareja, J. (2020). A Narrative Systematic Review of the Clinical Utility of Cycle Threshold Values in the Context of COVID-19. Infect. Dis. Ther. 9, 573-586.
Rashed, M. Z., Kopechek, J. A., Priddy, M. C., Hamorsky, K. T., Palmer, K. E., Mittal, N., Valdez, J., Flynn, J., and Williams, S. J. (2021). Rapid detection of SARS-CoV-2 antibodies using electrochemical impedance-based detector. Biosens. Bioelectron. 171, 112709.
Rath, S. L., and Kumar, K. (2020). Investigation of the Effect of Temperature on the Structure of SARS-CoV-2 Spike Protein by Molecular Dynamics Simulations. Front. Mol. Biosci. 7.
Seo, G., Lee, G., Kim, M. J., Baek, S.-H., Choi, M., Ku, K. B., Lee, C.-S., Jun, S., Park, D., Kim, H. G., et al. (2020). Rapid Detection of COVID-19 Causative Virus (SARS-CoV-2) in Human Nasopharyngeal Swab Specimens Using Field-Effect Transistor-Based Biosensor. ACS Nano 14, 5135-5142.
Service, R. (2020). Coronavirus antigen tests: quick and cheap, but too often wrong? Science (80-.).
Sorokina, M., M. C. Teixeira, J., Barrera-Vilarmau, S., Paschke, R., Papasotiriou, I., Rodrigues, J. P. G. L. M., and Kastritis, P. L. (2020). Structural models of human ACE2 variants with SARS-CoV-2 Spike protein for structure-based drug design. Sci. Data 7, 309.
The New York Times (2020). Coronavirus Map: Tracking the Global Outbreak.
Torrente-Rodriguez, R. M., Lukas, H., Tu, J., Min, J., Yang, Y., Xu, C., Rossiter, H. B., and Gao, W. (2020). SARS-CoV-2 RapidPlex: A Graphene-Based Multiplexed Telemedicine Platform for Rapid and Low-Cost COVID-19 Diagnosis and Monitoring. Matter 3, 1981-1998.
Turner, A. J. (2015). ACE2 Cell Biology, Regulation, and Physiological Functions. In The Protective Arm of the Renin Angiotensin System (RAS), (Elsevier), pp. 185-189.
Uhteg, K., Jarrett, J., Richards, M., Howard, C., Morehead, E., Geahr, M., Gluck, L., Hanlon, A., Ellis, B., Kaur, H., et al. (2020). Comparing the analytical performance of three SARS-CoV-2 molecular diagnostic assays. J. Clin. Virol. 127, 104384.
Uygun, Z. O., and Ertu\check{g}rul Uygun, H. D. (2014). A short footnote: Circuit design for faradaic impedimetric sensors and biosensors. Sensors Actuators B Chem. 202, 448-453.
Yakoh, A., Pimpitak, U., Rengpipat, S., Hirankarn, N., Chailapakul, O., and Chaiyo, S. (2021). Paper-based electrochemical biosensor for diagnosing COVID-19: Detection of SARS-CoV-2 antibodies and antigen. Biosens. Bioelectron. 176, 112912.
Yan, C., Cui, J., Huang, L., Du, B., Chen, L., Xue, G., Li, S., Zhang, W., Zhao, L., Sun, Y., et al. (2020). Rapid and visual detection of 2019 novel coronavirus (SARS-CoV-2) by a reverse transcription loop-mediated isothermal amplification assay. Clin. Microbiol. Infect. 26, 773-779.
Yang, J., Petitjean, S. J. L., Koehler, M., Zhang, Q., Dumitru, A. C., Chen, W., Derclaye, S., Vincent, S. P., Soumillion, P., and Alsteens, D. (2020). Molecular interaction and inhibition of SARS-CoV-2 binding to the ACE2 receptor. Nat. Commun. 11, 4541.
Zhao, H., Liu, F., Xie, W., Zhou, T.-C., OuYang, J., Jin, L., Li, H., Zhao, C.-Y., Zhang, L., Wei, J., et al. (2021). Ultrasensitive supersandwich-type electrochemical sensor for SARS-CoV-2 from the infected COVID-19 patients using a smartphone. Sensors Actuators B Chem. 327, 128899.
Zou, L., Ruan, F., Huang, M., Liang, L., Huang, H., Hong, Z., Yu, J., Kang, M., Song, Y., Xia, J., et al. (2020). SARS-CoV-2 Viral Load in Upper Respiratory Specimens of Infected Patients. N. Engl. J. Med. 382, 1177-1179.

What is claimed:

1. A device for assessing the presence of SARS-CoV-2 in a biological sample comprising:
a substrate comprising bacterial cellulose that includes a top surface and a back surface; and,
an electrode on the top surface of the substrate, wherein the electrode is functionalized with a detection moiety that binds SARS-CoV-2 spike protein;
a chemical cross linker comprising polyethylene glycol (PEG) that enables immobilization of the detection moiety that binds SARS-CoV-2 spike protein on the electrode.

2. The device according to claim 1, wherein the electrode is surface-functionalized with thiol or amine groups.

3. The device according to claim 1, wherein the detection moiety that binds SARS-CoV-2 spike protein is human Angiotensin Converting Enzyme 2 (ACE2), a fragment of ACE2, or an antibody.

4. The device according to claim 1, wherein the PEG is conjugated with graphene oxide.

5. The device according to claim 1, wherein ACE2 or the fragment thereof is immobilized on the electrode via an amide bond between the PEG and the ACE2 or the fragment thereof.

6. A method for assessing the presence of SARS-CoV-2 in a biological sample comprising:
contacting a device according to claim 1 with the biological sample;
exposing the device to an alternating current (AC) potential in order to generate a signal from the device;
and,
assessing the signal that is generated by the device electrochemical impedance spectroscopy (EIS) in order to determine the absence or presence of SARS-CoV-2 in the biological sample.

7. A device for assessing the presence of herpes simplex virus (HSV) in a biological sample comprising:
a substrate that includes a top surface and a back surface; and,
an electrode on the top surface of the substrate, wherein the electrode is functionalized with a detection moiety that binds HSV glycoprotein gD2.

8. The device according to claim 7, wherein the electrode is surface-functionalized with thiol or amine groups.

9. The device according to claim 7, wherein the detection moiety that binds HSV glycoprotein gD2 is nectin-1 or an antibody.

10. The device according to claim 7, comprising a chemical cross linker that enables immobilization of the detection moiety that binds HSV glycoprotein gD2 on the electrode.

11. The device according to claim 10, wherein nectin-1 is immobilized on the electrode via an amide bond between the chemical cross linker and nectin-1.

12. The device according to claim 7, further comprising a permselective membrane on the electrode.

13. A method for assessing the presence of HSV in a biological sample comprising:
contacting a device according to claim 7 with the biological sample;
exposing the device to an alternating current (AC) potential in order to generate a signal from the device;
and,
assessing the signal that is generated by the device electrochemical impedance spectroscopy (EIS) in order to determine the absence or presence of HSV in the biological sample.

14. A device for assessing the presence of SARS-CoV-2 in a biological sample comprising:
a substrate comprising a top surface and a back surface; and,
an electrode on the top surface of the substrate, wherein the electrode is functionalized with a detection moiety that binds SARS-CoV-2 spike protein.

15. The device according to claim 14, wherein the electrode is surface-functionalized with thiol or amine groups.

16. The device according to claim 14, wherein the detection moiety that binds SARS-CoV-2 spike protein is human Angiotensin Converting Enzyme 2 (ACE2), SEQ ID NO:1, or an antibody.

17. The device according to claim 14, wherein the detection moiety that binds SARS-CoV-2 spike protein is human Angiotensin Converting Enzyme 2 (ACE2).

18. The device according to claim 14, comprising a chemical cross linker that enables immobilization of the detection moiety that binds SARS-CoV-2 spike protein on the electrode.

19. The device according to claim 18, wherein the chemical cross linker is glutaraldehyde.

20. The device according to claim 19, wherein ACE2 or SEQ ID NO:1 is immobilized on the electrode via an amide bond between the glutaraldehyde and the N-terminus of ACE2 or SEQ ID NO:1.

21. The device according to claim 14, further comprising a permselective membrane on the electrode.

22. A method for assessing the presence of SARS-CoV-2 in a biological sample comprising:
contacting a device according to claim 14 with the biological sample;
exposing the device to an alternating current (AC) potential in order to generate a signal from the device;
and,
assessing the signal that is generated by the device electrochemical impedance spectroscopy (EIS) in order to determine the absence or presence of SARS-CoV-2 in the biological sample.
